(19) United States
(12) Patent Application Publication
(10) Pub. No.: US 2021/0261649 A1
(43) Pub. Date: Aug. 26, 2021

(54) COMPOSITIONS AND METHODS FOR ANTIBODY DELIVERY

(71) Applicant: Krystal Biotech, Inc., Pittsburgh, PA (US)

(72) Inventors: Trevor PARRY, San Diego, CA (US); Pooja AGARWAL, Mars, PA (US); Suma KRISHNAN, San Francisco, CA (US)

(73) Assignee: Krystal Biotech, Inc,, Pittsburgh, PA (US)

(21) Appl. No.: 17/255,383

(22) PCT Filed: Jun. 28, 2019
(86) PCT No.: PCT/US2019/039939

§ 371 Date: Dec. 22, 2020

Related U.S. Application Data
(60) Provisional application No. 62/713,066, filed on Aug. 1, 2018.
(60) Provisional application No. 62/692,514, filed on Jun. 29, 2018.

(51) Int. Cl. C07K 16/08; C12N 7/00

(52) U.S. Cl. CPC C07K 16/087; C12N 7/00; C07K 2317/569; C12N 2710/16643; C07K 2317/75; C07K 2317/76; C12N 2710/16662

(57) ABSTRACT

The present disclosure provides recombinant nucleic acids (e.g., recombinant herpes viral genomes) comprising one or more polynucleotides encoding an antibody; viruses (e.g., herpes viruses) comprising the recombinant nucleic acids; compositions comprising the recombinant nucleic acids and/or viruses; methods of their use (e.g., for localized, virus-mediated delivery and expression of the encoded antibody); and articles of manufacture or kits thereof.

### CROSS REFERENCE TO RELATED APPLICATIONS

This application claims the priority benefit of U.S. Provisional Application Ser. No. 62/692,514, filed Jun. 29, 2018, and U.S. Provisional Application Ser. No. 62/713,066, filed Aug. 1, 2018, which are each incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761342000840SEQLIST.txt, date recorded: Jun. 27, 2019, size: 764 KB).

### FIELD OF THE INVENTION

The present disclosure relates, in part, to recombinant nucleic acids (e.g., recombinant herpes viral genomes) comprising one or more polynucleotides encoding an antibody (or a portion thereof); to viruses (e.g., herpes viruses) comprising the recombinant nucleic acids; to compositions comprising the recombinant nucleic acids and/or viruses; to methods of their use (e.g., for localized, virus-mediated delivery and expression of the encoded antibody); and to articles of manufacture or kits thereof.

### BACKGROUND

In recent years, therapeutic antibodies have become one of the commercially most successful classes of biopharmaceutical drugs. While antibodies have shown success in the treatment of several major diseases including autoimmune, cardiovascular, and infectious diseases, cancer, and inflammation, systemic administration of therapeutic antibodies has a number of functional limitations, including inadequate pharmacokinetics and tissue accessibility. In addition, systemic exposure to certain antibodies has been shown to repress the immune system, exposing the patient to significant risk of infections and other complications. Accordingly, there is need for alternative strategies to administer therapeutic antibodies to patients in need thereof.

All references cited herein, including patent applications, patent publications, non-patent literature, and UniProtKB/Swiss-Prot Accession numbers are herein incorporated by reference in their entirety, as if each individual reference were specifically and individually indicated to be incorporated by reference.

### BRIEF SUMMARY

In some embodiments, provided herein are recombinant nucleic acids (e.g., recombinant herpes viral genomes) encoding antibodies (e.g., full-length antibodies, antibody fragments, etc.) for use in viruses (e.g., herpes viruses), compositions, formulations, medicaments, and/or methods useful for delivering (e.g., topically, intraarticularly, intravitreally, etc.) antibodies to one or more sites and/or tissues of a subject. The present inventors have shown that the recombinant, attenuated viruses described herein were capable of 1) expressing both full-length antibodies and antibody fragments (scFv-Fcs), 2) encoding and delivering mouse, chimeric, and fully human antibodies (of various IgG isotypes), and 3) inducing dose-dependent secretion of the encoded antibodies from human cells, which were functional (see e.g., Example 2). Moreover, the present inventors have shown that the viruses described herein may be used to successfully express their encoded antibodies in vivo after localized administration (see e.g., Example 3). Furthermore, the present inventors have shown that the viruses described herein may be used to successfully express a therapeutic antibody to treat one or more signs of an inflammatory skin condition (atopic dermatitis) after topical administration (see e.g., Example 3). Without wishing to be bound by theory, it is believed that the recombinant nucleic acids (e.g., recombinant viral genomes), viruses, pharmaceutical compositions, medicaments, and/or methods described herein provide a novel system for delivering therapeutic antibodies to a patient. Specifically, without wishing to be bound by theory, it is believed that the recombinant herpes viruses described herein provide a unique system to locally administer a therapeutic antibody to a subject in order to: 1) improve antibody pharmacokinetics at the site of interest; 2) increase antibody tissue accessibility and/or infiltration; 3) reduce the total dose of the antibody administered to the subject; 4) provide a less invasive or non-invasive method of administering an antibody to the subject; and/or 5) reduce or eliminate systemic exposure of the subject to the antibody (e.g., to avoid one or more side-effects (such as global immune suppression) observed after systemic administration of certain antibodies).

Accordingly, certain aspects of the present disclosure relate to a recombinant herpes virus genome comprising one or more polynucleotides encoding an antibody. In some embodiments, the antibody is an antibody fragment. In some embodiments, the antibody fragment is a Fab, Fab′ Fab′-SH, F(ab′)2, Fv, scFv, or scFv-Fc fragment. In some embodiments, the antibody fragment is an scFv-Fc fragment. In some embodiments, the scFv-Fc comprises the Fc region of an IgG antibody (e.g., the Fc region of an IgG1, IgG2, IgG3, or IgG4 antibody). In some embodiments, the scFv-Fc comprises the Fc region of an IgG1 antibody. In some embodiments, the scFv-Fc comprises the Fc region of an IgG4 antibody. In some embodiments, the antibody is a full-length antibody. In some embodiments that may be combined with any of the preceding embodiments, the antibody is a murine antibody, a chimeric antibody, a humanized antibody, a human antibody, a monoclonal antibody, or a multispecific antibody. In some embodiments that may be combined with any of the preceding embodiments, the antibody is an IgA, IgD, IgE, IgG, or IgM antibody. In some embodiments that may be combined with any of the preceding embodiments, the antibody is an IgG antibody. In some embodiments, the IgG antibody is an IgG1, IgG2, IgG3, or IgG4 antibody. In some embodiments, the IgG antibody is an IgG1 antibody. In some embodiments, the IgG antibody is an IgG4 antibody. In some embodiments that may be combined with any of the preceding embodiments, the antibody is an agonist antibody or an antagonist antibody. In some embodiments, the antibody is an agonist antibody. In some embodiments, the antibody is an antagonist antibody.

In some embodiments that may be combined with any of the preceding embodiments, the antibody comprises a heavy chain variable region comprising an HVR-H1, an HVR-H2, and an HVR-H3, wherein the HVR-H1 comprises a sequence selected from SEQ ID NOS: 1-59, the HVR-H2 comprises a sequence selected from SEQ ID NOS: 60-122, and/or the HVR-H3 comprises a sequence selected from SEQ ID NOS: 123-185. In some embodiments that may be combined with any of the preceding embodiments, the antibody comprises a light chain variable region comprising an HVR-L1, an HVR-L2, and an HVR-L3, wherein the HVR-L1 comprises a sequence selected from SEQ ID NOS: 186-242, the HVR-L2 comprises a sequence selected from SEQ ID NOS: 243-294, and/or the HVR-L3 comprises a sequence selected from SEQ ID NOS: 295-354.

In some embodiments that may be combined with any of the preceding embodiments, the antibody comprises a heavy chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 355-419 or 614-865. In some embodiments that may be combined with any of the preceding embodiments, the antibody comprises a light chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 420-482 or 866-1116. In some embodiments that may be combined with any of the preceding embodiments, the antibody comprises: (a) a heavy chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 355-419 or 614-865; and (b) a light chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 420-482 or 866-1116.

In some embodiments that may be combined with any of the preceding embodiments, the antibody is selected from abagovomab, abciximab, abituzumab, abrezekimab, abrilumab, actoxumab, adalimumab, adecatumumab, aducanumab, afasevikumab, afelimomab, afutuzumab, alacizumab, alemtuzumab, alirocumab, altumomab, amatuximab, anatumomab, andecaliximab, anetumab, anifrolumab, anrukinzumab, apolizumab, aprutumab, arcitumomab, ascrinvacumab, aselizumab, atezolizumab, atinumab, atlizumab, atorolimumab, avelumab, azintuxizumab, bapineuzumab, basiliximab, bavituximab, bectumomab, begelomab, belantamab, belimumab, bemarituzumab, belimumab, bemaritzumab, benralizumab, berlimatoxumab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab, briakinumab, brodalumab, brolucizumab, brontictuzumab, burosumab, cabiralizumab, camidanlumab, camrelizumab, canakinumab, cantuzumab, caplacizumab, capromab, carlumab, carotuximab, catumaxomab, cedelizumab, cemiplimab, cergutuzumab, certolizumab, cetrelimab, cetuximab, cibisatamab, citatuzumab, cixutumumab, clazakizumab, clenoliximab, clivatuzumab, codrituzumab, cofetuzumab, coltuximab, conatumumab, concizumab, cosfroviximab, crenezumab, crizanlizumab, crotedumab, cusatuzumab, dacetuzumab, daclizumab, dalotuzumab, dapirolizumab, daratumumab, dectrekumab, demcizumab, denintuzumab, denosumab, depatuxizumab, derlotuximab, detumomab, dezamizumab, dinutuximab, diridavumab, domagrozumab, dorlimomab, drozitumab, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab, enavatuzumab, enfortumab, enlimomab, enoblituzumab, enokizumab, enoticumab, ensituximab, epitumomab, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, felvizumab fezakinumab, fibatuzumab, ficlatuzumab, figitumumab, firivumab, flanvotumab, fletikumab, flotetuzumab, fontolizumab, foralumab, foravirumab, fremanezumab, fresolimumab, frunevetmab, fulranumab, futuximab, galcanezumab, galiximab, gancotamab, ganitumab, gantenerumab, gatipotuzumab, gavilimomab, gedivumab, gemtuzumab, gevokizumab, gilvetmab, gimsilumab, girentuximab, glembatumumab, golimumab, gomiliximab, gosuranemab, guselkumab, ianalumab, ibalizumab, ibritumomab, icrucumab, idarucizumab, ifabotuzumab, igovomab, iladatuzumab, imalumab, imaprelimab, imciromab, imgatuzumab, inclacumab, indatuximab, indusatumab, inebilizumab, inflectra, infliximab, intetumumab, inolimomab, inotuzumab, ipilimumab, iratumumab, isatuximab, iscalimab, istiratumab, itolizumab, ixekizumab, keliximab, labetuzumab, lacnotuzumab, ladiratuzumab, lampalizumab, lanadelumab, landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, loncastuximab, losatuxizumab, lilotomab, lintuzumab, lirilumab, lodelcizumab, lokivetmab, lorvotuzumab, lucatumumab, lulizumab, lumiliximab, lumretuzumab, lupartumab, lutikizumab, mapatumumab, margetuximab, marstacimab, maslimomab, mavrilimumab, matuzumab, mepolizumab, metelimumab, milatuzumab, minretumomab, mirikizumab, mirvetuximab, mitumomab, modotuximab, mogamulizumab, monalizumab, morolimumab, mosunetuzumab, motavizumab, moxetumomab, nacolomab, namilumab, naptumomab, naratuximab, narnatumab, natalizumab, navicixizumab, navivumab, naxitamab, nebacumab, necitumumab, nemolizumab, nerelimomab, nesvacumab, netakimab, nimotuzumab, nirsevimab, nivolumab, nofetumomab, obiltoxaximab, obinutuzumab, ocaratuzumab, ocrelizumab, odulimomab, ofatumumab, olaratumab, oleclumab, olendalizumab, olokizumab, omalizumab, onartuzumab, ontuxizumab, onvatilimab, opicinumab, oportuzumab, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab, pintumomab, placulumab, plozalizumab, pogalizumab, polatuzumab, ponezumab, porgaviximab, prasinezumab, prezalizumab, priliximab, pritoxaximab, pritumumab, quilizumab, racotumomab, radretumab, rafivirumab, ralpancizumab, ramucirumab, ranevetmab, ranibizumab, raxibacumab, ravagalimab, ravulizumab, refanezumab, regavirumab, remtolumab, reslizumab, rilotumumab, rinucumab, risankizumab, rituximab, rivabazumab, robatumumab, roledumab, romilkimab, romosozumab, rontalizumab, rosmantuzumab, rovalpituzumab, rovelizumab, rozanolixizumab, ruplizumab, sacituzumab, samalizumab, samrotamab, sapelizumab, sarilumab, satralizumab, satumomab, secukinumab, selicrelumab, seribantumab, setoxaximab, setrusumab, sevirumab, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, sulesomab, suptavumab, sutimlimab, suvizumab, suvratoxumab, tabalumab, tacatuzumab, tadocizumab, talacotuzumab, talizumab, tamtuvetmab, tanezumab, taplitumomab, tarextumab, tavolimab, tefibazumab, telimomab, telisotuzumab, tenatumomab, teneliximab, teplizumab, tepoditamab, teprotumumab, tesidolumab, tetulomab, tezepelumab, tibulizumab, tildrakizumab, tigatuzumab, timigutuzumab, timolumab, tiragotumab, tislelizumab, tisotumab, tocilizumab, tomuzotuximab, toralizumab, tosatoxumab, tositumomab, tovetumab, tralokinumab, trastuzumab, tregalizumab, tremelimumab, trevogrumab, tucotuzumab, tuvirumab, ublituximab, ulocuplumab, urelumab, urtoxazumab, ustekinumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab, votumumab, vunakizumab, xentuzumab, zalutumumab, zanolimumab, zatuximab, zenocutuzumab, ziralimumab, zolbetuximab, and zolimomab.

In some embodiments that may be combined with any of the preceding embodiments, the recombinant herpes virus genome is replication competent. In some embodiments that may be combined with any of the preceding embodiments, the recombinant herpes virus genome is replication defective. In some embodiments that may be combined with any of the preceding embodiments, the recombinant herpes virus genome is selected from a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any derivatives thereof.

In some embodiments that may be combined with any of the preceding embodiments, the recombinant herpes virus genome is a recombinant herpes simplex virus genome. In some embodiments, the recombinant herpes simplex virus genome is a recombinant type 1 herpes simplex virus (HSV-1) genome, a recombinant type 2 herpes simplex virus (HSV-2) genome, or any derivatives thereof. In some embodiments, the recombinant herpes simplex virus genome is a recombinant type 1 herpes simplex virus (HSV-1) genome. In some embodiments, the recombinant herpes simplex virus genome comprises an inactivating mutation. In some embodiments, the inactivating mutation is in a herpes simplex virus gene. In some embodiments, the inactivating mutation is a deletion of the coding sequence of the herpes simplex virus gene. In some embodiments, the herpes simplex virus gene is selected from Infected Cell Protein (ICP) 0, ICP4, ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivating mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivating mutation in the UL41 gene. In some embodiments that may be combined with any of the preceding embodiments, the recombinant herpes simplex virus genome comprises an inactivating mutation in one or both copies of the ICP0 gene. In some embodiments that may be combined with any of the preceding embodiments, the recombinant herpes simplex virus genome comprises an inactivating mutation in the ICP27 gene. In some embodiments that may be combined with any of the preceding embodiments, the recombinant herpes simplex virus genome comprises an inactivating mutation in the UL55 gene. In some embodiments that may be combined with any of the preceding embodiments, the recombinant herpes simplex virus genome comprises an inactivating mutation in the Joint region. In some embodiments, the recombinant herpes simplex virus genome comprises a deletion of the Joint region. In some embodiments that may be combined with any of the preceding embodiments, the recombinant herpes simplex virus genome comprises the one or more polynucleotides within one or both of the ICP4 viral gene loci.

In some embodiments that may be combined with any of the preceding embodiments, the recombinant herpes virus genome has reduced cytotoxicity when introduced into a target cell, as compared to a corresponding wild-type herpes virus genome. In some embodiments, the target cell is a human cell.

Other aspects of the present disclosure relate to a herpes virus comprising any of the recombinant herpes virus genomes described herein. In some embodiments, the herpes virus is replication competent. In some embodiments, the herpes virus is replication defective. In some embodiments, the herpes virus is attenuated. In some embodiments that may be combined with any of the preceding embodiments, the herpes virus has reduced cytotoxicity as compared to a corresponding wild-type herpes virus. In some embodiments that may be combined with any of the preceding embodiments, the herpes virus is selected from a herpes simplex virus, a varicella zoster virus, a human cytomegalovirus, a herpesvirus 6A, a herpesvirus 6B, a herpesvirus 7, and a Kaposi's sarcoma-associated herpesvirus. In some embodiments that may be combined with any of the preceding embodiments, the herpes virus is a herpes simplex virus. In some embodiments, the herpes simplex virus is a type 1 herpes simplex virus (HSV-1), a type 2 herpes simplex virus (HSV-2), or any derivatives thereof. In some embodiments, the herpes simplex virus is a type 1 herpes simplex virus (HSV-1).

Other aspects of the present disclosure relate to a pharmaceutical composition comprising: (a) any of the recombinant herpes virus genomes described herein and/or any of the herpes viruses described herein; and (b) a pharmaceutically acceptable excipient. In some embodiments, the pharmaceutical composition is suitable for topical, transdermal, subcutaneous, intradermal, transmucosal, oral, intranasal, intratracheal, sublingual, nasal, buccal, rectal, vaginal, intravenous, intraarterial, intramuscular, intracardiac, intraosseous, intraperitoneal, intraorbital, intravitreal, subconjunctival, suprachoroidal, subretinal, intraarticular, peri-articular, local, epicutaneous, and/or inhaled administration. In some embodiments, the pharmaceutical composition is suitable for topical administration. In some embodiments, the pharmaceutical composition is suitable for inhaled administration. In some embodiments, the pharmaceutical composition is suitable for injection.

Other aspects of the present disclosure relate to the use of any of the recombinant herpes virus genomes, herpes viruses, and/or pharmaceutical compositions described herein as a medicament.

Other aspects of the present disclosure relate to the use of any of the recombinant herpes virus genomes, herpes viruses, and/or pharmaceutical compositions described herein in a therapy.

Other aspects of the present disclosure relate to the use of any of the recombinant herpes virus genomes, herpes viruses, and/or pharmaceutical compositions described herein in the manufacture of a medicament for treating a disease. In some embodiments, the disease is an inflammatory skin disease (e.g., atopic dermatitis). In some embodiments, the disease is selected from psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer, hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, an autoimmune disease, asthma, thyroid eye disease, an infectious disease, and a neurological disease.

Other aspects of the present disclosure relate to a method of administering an antibody to a subject comprising administering to the subject an effective amount of any of the herpes viruses described herein and/or any of the pharmaceutical compositions described herein. In some embodiments, the herpes virus or pharmaceutical composition is administered topically, transdermally, subcutaneously, intradermally, transmucosally, orally, intranasally, intratracheally, sublingually, nasally, buccally, rectally, vaginally, intravenously, intraarterially, intramuscularly, intracardially, intraosseously, intraperitoneally, intraorbitally, intravitreally, subconjunctivally, suprachoroidally, subretinally, intraarticularly, peri-articularly, locally, epicutaneously, or via inhalation.

Other aspects of the present disclosure relate to a method of providing prophylactic, palliative, and/or therapeutic relief of one or more signs or symptoms of a disease in a subject comprising administering to the subject an effective amount of any of the herpes viruses described herein and/or any of the pharmaceutical compositions described herein. In some embodiments, the herpes virus or pharmaceutical composition is administered topically, transdermally, subcutaneously, intradermally, transmucosally, orally, intranasally, intratracheally, sublingually, nasally, buccally, rectally, vaginally, intravenously, intraarterially, intramuscularly, intracardially, intraosseously, intraperitoneally, intraorbitally, intravitreally, subconjunctivally, suprachoroidally, subretinally, intraarticularly, peri-articularly, locally, epicutaneously, or via inhalation. In some embodiments that may be combined with any of the preceding embodiments, the disease is selected from psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer, hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, asthma, an autoimmune disease, thyroid eye disease, an infectious disease, and a neurological disease.

Other aspects of the present disclosure relate to a method of an antibody to the epidermis and/or dermis of a subject comprising topically, transdermally, subcutaneously, or intradermally administering to the subject an effective amount of any of the herpes viruses described herein and/or any of the pharmaceutical compositions described herein. In some embodiments, the skin of the subject is abraded or made more permeable prior to administration.

Other aspects of the present disclosure relate to a method of administering an antibody to the mucosa of a subject comprising topically, transmucosally, orally, sublingually, nasally, intranasally, via inhalation, or buccally administering to the subject an effective amount any of the herpes viruses described herein and/or any of the pharmaceutical compositions described herein.

Other aspects of the present disclosure relate to a method of administering an antibody to the airway and/or lungs of a subject comprising orally, sublingually, nasally, intranasally, intratracheally, via inhalation, or buccally administering to the subject an effective amount of any of the herpes viruses described herein and/or any of the pharmaceutical compositions described herein.

Other aspects of the present disclosure relate to a method of administering an antibody to one or more joints of a subject comprising intraarticularly and/or peri-articularly administering to the subject an effective amount any of the herpes viruses described herein and/or any of the pharmaceutical compositions described herein.

Other aspects of the present disclosure relate to a method of administering an antibody to one or both eyes of a subject comprising topically, intraorbitally, intravitreally, subconjunctivally, subretinally, or suprachoroidally administering to the subject an effective amount of any of the herpes viruses described herein and/or any of the pharmaceutical compositions described herein.

In some embodiments that may be combined with any of the preceding embodiments, the subject is a human. In some embodiments that may be combined with any of the preceding embodiments, the subject is not exposed to the antibody systemically.

Other aspects of the present disclosure relate to a recombinant herpes simplex virus (HSV) genome comprising one or more polynucleotides encoding an antibody. In some embodiments, the antibody is an antibody fragment. In some embodiments, the antibody fragment is a Fab, Fab′ Fab′-SH, F(ab′)2, Fv, scFv, or scFv-Fc fragment. In some embodiments, the antibody is a full-length antibody.

In some embodiments that may be combined with any of the preceding embodiments, the antibody is a murine antibody, a chimeric antibody, a humanized antibody, a human antibody, a monoclonal antibody, or a multispecific antibody. In some embodiments that may be combined with any of the preceding embodiments, the antibody is an IgA, IgD, IgE, IgG, or IgM antibody. In some embodiments that may be combined with any of the preceding embodiments, the antibody is an IgG antibody. In some embodiments, the IgG antibody is an IgG1, IgG2, IgG3, or IgG4 antibody. In some embodiments, the antibody is an agonist antibody. In some embodiments, the antibody is an antagonist antibody.

In some embodiments that may be combined with any of the preceding embodiments, the antibody comprises a heavy chain variable region comprising an HVR-H1, an HVR-H2, and an HVR-H3, wherein the HVR-H1 comprises a sequence selected from the group consisting of SEQ ID NOS: 1-59, the HVR-H2 comprises a sequence selected from the group consisting of SEQ ID NOS: 60-122, and/or the HVR-H3 comprises a sequence selected from the group consisting of SEQ ID NOS: 123-185. In some embodiments, the heavy chain variable region comprises a sequence having at least 85%, at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOS: 355-419. In some embodiments that may be combined with any of the preceding embodiments, the antibody comprises a light chain variable region comprising an HVR-L1, an HVR-L2, and an HVR-L3, wherein the HVR-L1 comprises a sequence selected from the group consisting of SEQ ID NOS: 186-242, the HVR-L2 comprises a sequence selected from the group consisting of SEQ ID NOS: 243-294, and/or the HVR-L3 comprises a sequence selected from the group consisting of SEQ ID NOS: 395-354. In some embodiments, the light chain variable region comprises a sequence having at least 85%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 420-482.

In some embodiments that may be combined with any of the preceding embodiments, the recombinant genome is a recombinant HSV-1 genome, a recombinant HSV-2 genome, or any derivatives thereof. In some embodiments that may be combined with any of the preceding embodiments, the recombinant genome comprises an inactivating mutation in a herpes simplex virus gene. In some embodiments, the herpes simplex virus gene is selected from the group consisting of Infected Cell Protein (ICP) 0, ICP4, ICP22, ICP27, ICP47, thymidine kinase (tk), Long Unique Region (UL) 41, and UL55. In some embodiments, the recombinant genome comprises an inactivation mutation in one or both copies of the ICP4 gene. In some embodiments, the recombinant genome comprises an inactivating mutation in the ICP22 gene. In some embodiments, the recombinant genome comprises an inactivating mutation in the UL41 gene. In some embodiments, the recombinant genome comprises an inactivating mutation in the ICP0 gene. In some embodiments, the recombinant genome comprises an inactivating mutation in the ICP27 gene. In some embodiments, the inactivating mutation is a deletion of the coding sequence of the gene(s). In some embodiments that may be combined with any of the preceding embodiments, the recombinant genome has reduced cytotoxicity when introduced into a target cell as compared to a wild-type herpes simplex virus genome. In some embodiments, the target cell is a human cell. In some embodiments, the target cell is a keratinocyte or fibroblast.

In some embodiments that may be combined with any of the preceding embodiments, the recombinant genome comprises the one or more polynucleotides within one or more viral gene loci. In some embodiments, the recombinant genome comprises the one or more polynucleotides within one or both of the ICP4 viral gene loci. In some embodiments, the recombinant genome comprises the one or more polynucleotides within the ICP22 viral gene locus. In some embodiments, the recombinant genome comprises the one or more polynucleotides within the UL41 viral gene locus.

Other aspects of the present disclosure relate to a herpes simplex virus (HSV) comprising any of the recombinant genomes described herein. In some embodiments, the HSV is replication competent. In some embodiments, the HSV is replication defective. In some embodiments that may be combined with any of the preceding embodiments, the HSV has reduced cytotoxicity as compared to a wild-type herpes simplex virus. In some embodiments that may be combined with any of the preceding embodiments, the HSV is a herpes simplex type 1 virus, a herpes simplex type 2 virus, or any derivatives thereof.

Other aspects of the present disclosure relate to a pharmaceutical composition comprising any of the recombinant genomes and/or viruses described herein and a pharmaceutically acceptable excipient. In some embodiments, the pharmaceutical composition is suitable for topical, transdermal, subcutaneous, intradermal, transmucosal, sublingual, nasal, buccal, intraorbital, intravitreal, subconjunctival, suprachoroidal, intraarticular, and/or inhaled administration. In some embodiments, the pharmaceutical composition is suitable for topical administration. In some embodiments that may be combined with any of the preceding embodiments, the pharmaceutical composition comprises a hydroxypropyl methylcellulose gel. In some embodiments that may be combined with any of the preceding embodiments, the pharmaceutical composition comprises a phosphate buffer. In some embodiments that may be combined with any of the preceding embodiments, the pharmaceutical composition comprises glycerol. In some embodiments that may be combined with any of the preceding embodiments, the pharmaceutical composition comprises a lipid carrier. In some embodiments that may be combined with any of the preceding embodiments, the pharmaceutical composition comprises a nanoparticle carrier.

Other aspects of the present disclosure relate to a method of administering an antibody to a subject comprising administering to the subject an effective amount of any of the viruses or pharmaceutical compositions described herein. In some embodiments, the virus or composition is administered topically, transdermally, subcutaneously, intradermally, transmucosally, sublingually, nasally, buccally, intravitreally, subconjunctivally, suprachoroidally, intraarticularly, or via inhalation. In some embodiments that may be combined with any of the preceding embodiments, the subject is a human. In some embodiments that may be combined with any of the preceding embodiments, the subject is not exposed to the antibody systemically.

Other aspects of the present disclosure relate to a method of providing prophylactic, palliative, and/or therapeutic relief of one or more signs or symptoms of a disease in a subject comprising administering to the subject and effective amount of any of the viruses or pharmaceutical compositions described herein. In some embodiments, the virus or composition is administered topically, transdermally, subcutaneously, intradermally, transmucosally, sublingually, nasally, buccally, intravitreally, subconjunctivally, suprachoroidally, intraarticularly, or via inhalation. In some embodiments that may be combined with any of the preceding embodiments, the disease is selected from the group consisting of psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer, hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, autoimmune disease, melanoma, uveal melanoma, and thyroid eye disease. In some embodiments, the disease is not cancer. In some embodiments that may be combined with any of the preceding embodiments, the subject is a human. In some embodiments that may be combined with any of the preceding embodiments, the subject is not exposed to the antibody systemically.

Other aspects of the present disclosure relate to a method of administering an antibody to the epidermis and/or dermis of a subject comprising topically, transdermally, or intradermally administering to the subject an effective amount of any of the viruses or pharmaceutical compositions described herein. In some embodiments, the skin of the subject is abraded prior to administration. In some embodiments that may be combined with any of the preceding embodiments, the subject is a human. In some embodiments that may be combined with any of the preceding embodiments, the subject is not exposed to the antibody systemically.

Other aspects of the present disclosure relate to a method of administering an antibody to the mucosa of a subject comprising topically, transmucosally, sublingually, nasally, or buccally administering to the subject an effective amount of any of the viruses or pharmaceutical compositions described herein. In some embodiments, the subject is a human. In some embodiments that may be combined with any of the preceding embodiments, the subject is not exposed to the antibody systemically.

Other aspects of the present disclosure relate to a method of administering an antibody to one or more joints of a subject comprising intraarticularly administering to the subject an effective amount of any of the viruses or pharmaceutical compositions described herein. In some embodiments, the subject is a human. In some embodiments that may be combined with any of the preceding embodiments, the subject is not exposed to the antibody systemically.

Other aspects of the present disclosure relate to a method of administering an antibody to one or both eyes of a subject comprising topically, intraorbitally, intravitreally, subconjunctivally, or suprachoroidally administering to the subject an effective amount of any of the viruses or pharmaceutical compositions described herein. In some embodiments, the subject is a human. In some embodiments that may be combined with any of the preceding embodiments, the subject is not exposed to the antibody systemically.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-N show schematics of wild-type and modified herpes simplex virus genomes. FIG. 1A shows a wild-type herpes simplex virus genome. FIG. 1B shows a modified herpes simplex virus genome comprising deletions of the coding sequences of ICP4 (both copies) and ICP22, with a polynucleotide containing the coding sequence of a single-chain antibody (an scFv-Fc) operably linked to a heterologous promoter integrated at each of the ICP4 loci. FIG. 1C shows a modified herpes simplex virus genome comprising deletions of the coding sequence of ICP4 (both copies), with a polynucleotide containing the coding sequence of an scFv-Fc operably linked to a heterologous promoter integrated at each of the ICP4 loci. FIG. 1D shows a modified herpes simplex virus genome comprising deletions of the coding sequences of ICP4 (both copies) and ICP22, with a polynucleotide containing 1) the coding sequence of an antibody heavy chain operably linked to a first heterologous promoter, and 2) the coding sequence of an antibody light chain operably linked to a second heterologous promoter, integrated at each of the ICP4 loci. Both the antibody heavy and light chains are encoded on the same strand of DNA. FIG. 1E shows a modified herpes simplex virus genome comprising deletions of the coding sequence of ICP4 (both copies), with a polynucleotide containing 1) the coding sequence of an antibody heavy chain operably linked to a first heterologous promoter, and 2) the coding sequence of an antibody light chain operably linked to a second heterologous promoter, integrated at each of the ICP4 loci. Both the antibody heavy and light chains are encoded on the same strand of DNA. FIG. 1F shows a modified herpes simplex virus genome comprising deletions of the coding sequences of ICP4 (both copies) and ICP22, with a polynucleotide containing 1) the coding sequence of an antibody heavy chain operably linked to a first heterologous promoter, and 2) the coding sequence of an antibody light chain operably linked to a second heterologous promoter, integrated at each of the ICP4 loci. The antibody heavy and light chains are encoded on opposite strands of DNA. FIG. 1G shows a modified herpes simplex virus genome comprising deletions of the coding sequence of ICP4 (both copies), with a polynucleotide containing 1) the coding sequence of an antibody heavy chain operably linked to a first heterologous promoter, and 2) the coding sequence of an antibody light chain operably linked to a second heterologous promoter, integrated at each of the ICP4 loci. The antibody heavy and light chains are encoded on opposite strands of DNA. FIG. 1H shows a modified herpes simplex virus genome comprising deletions of the coding sequences of ICP4 (both copies) and ICP22, with a polynucleotide encoding a polycistronic mRNA operably linked to a heterologous promoter integrated at each of the ICP4 loci. The polycistronic mRNA contains the coding sequence of an antibody heavy chain and an antibody light chain separated by an internal ribosomal entry site (IRES). FIG. 1I shows a modified herpes simplex virus genome comprising deletions of the coding sequence of ICP4 (both copies), with a polynucleotide encoding a polycistronic mRNA operably linked to a heterologous promoter integrated at each of the ICP4 loci. The polycistronic mRNA contains the coding sequence of an antibody heavy chain and an antibody light chain separated by an internal ribosomal entry site (IRES). FIG. 1J shows a modified herpes simplex virus genome comprising deletions of the coding sequences of ICP4 (both copies), ICP22, and UL41, with a first polynucleotide containing the coding sequence of an antibody heavy chain operably linked to a heterologous promoter integrated at each of the ICP4 loci, and a second polynucleotide containing the coding sequence of an antibody light chain operably linked to a heterologous promoter integrated at the UL41 and ICP22 loci. FIG. 1K shows a modified herpes simplex virus genome comprising deletions of the coding sequences of ICP4 (both copies), ICP22, and UL41, with a first polynucleotide containing the coding sequence of an antibody light chain operably linked to a heterologous promoter integrated at each of the ICP4 loci, and a second polynucleotide containing the coding sequence of an antibody heavy chain operably linked to a heterologous promoter integrated at the UL41 and ICP22 loci. FIG. 1L shows a modified herpes simplex virus genome comprising deletions of the coding sequences of ICP4 (both copies) and UL41, with a first polynucleotide containing the coding sequence of an antibody heavy chain operably linked to a heterologous promoter integrated at each of the ICP4 loci, and a second polynucleotide containing the coding sequence of a polycistronic mRNA operably linked to a heterologous promoter integrated at the UL41 locus. The polycistronic mRNA contains two copies of the coding sequence of an antibody light chain separated by an internal ribosomal entry site (IRES). FIG. 1M shows a modified herpes simplex virus genome comprising deletions of the coding sequences of ICP4 (both copies) and ICP22, with a first polynucleotide containing the coding sequence of an antibody heavy chain operably linked to a heterologous promoter integrated at each of the ICP4 loci, and a second polynucleotide containing the coding sequence of a polycistronic mRNA operably linked to a heterologous promoter integrated at the ICP22 locus. The polycistronic mRNA contains two copies of the coding sequence of an antibody light chain separated by an internal ribosomal entry site (IRES). FIG. 1N shows a modified herpes simplex virus genome comprising deletions of the coding sequences of ICP4 (both copies), ICP22, and UL41, with a first polynucleotide containing the coding sequence of an antibody heavy chain operably linked to a heterologous promoter integrated at the ICP22 locus, and a second polynucleotide containing the coding sequence of an antibody light chain operably linked to a heterologous promoter integrated at the UL41 locus.

FIGS. 2A-B show the antibody concentration in cell supernatants harvested from mock infected (MOI-0) immortalized human keratinocytes (HaCaTs), or HaCaTs infected at the indicated multiplicities of infection (MOI) with engineered HSV vectors encoding the indicated antibodies, as assessed by ELISA. FIG. 2A shows the antibody concentration in cell supernatants harvested from HaCaT cells infected with HSV encoding a human (Ab1Fc1 or Ab1Fc2) or chimeric (Ab2Fc2) single-chain antibody. FIG. 2B shows the antibody concentration in cell supernatants harvested from HaCaT cells infected with HSV encoding a mouse (Ab66Fc1 or Ab66Fc2) single-chain antibody. For each condition, data is presented for two replicates ±SEM.

FIG. 3 shows the detectable levels of recombinant human TNFα spiked into cell supernatants harvested from mock infected (MOI 0) immortalized human keratinocytes (HaCaTs), or HaCaTs infected at the indicated multiplicities of infection (MOI) with an engineered HSV vector encoding an anti-TNFα human single-chain antibody (Ab1Fc1), as assessed by ELISA. For each condition, data is presented for two replicates ±SEM.

FIGS. 4A-B show the relative fold change vs. ethanol (EtOH) control in transcript levels of certain markers of atopic dermatitis-like lesions in mouse ear and dorsal skin treated topically for five days with the vitamin D3 synthetic analog calcipotriol (MC903), as assessed by qRT-PCR analysis. FIG. 4A shows the relative fold change in TSLP transcripts vs. EtOH control in mouse ear and dorsal skin treated topically with MC903 on Days 1-5, with tissues being harvested on Day 5 or Day 7. FIG. 4B shows the relative fold change in IL-4 transcripts vs. EtOH control in mouse ear and dorsal skin treated topically with MC903 on Days 1-5, with tissues being harvested on Day 5, Day 7, or Day 9.

FIG. 5 show the histology of representative mouse ear skin treated with MC903 or EtOH control, as assessed by hematoxylin and eosin (H&E) staining.

FIGS. 6A-B show representative immunofluorescence images of human single-chain antibody (Ab1Fc1) expression in ear and dorsal skin biopsies harvested from MC903-exposed C57BL/6J mice treated topically with either HSV-Ab1Fc1 or a negative control (vehicle). DAPI staining was used to visualize nuclei. FIG. 6A shows Ab1Fc1 expression in mouse ear and dorsal skin treated with MC903 on Days 1-5 and topical HSV-Ab1Fc1 (or vehicle control) on Day 5, with tissues being harvested on Day 7. FIG. 6B shows Ab1Fc1 expression in mouse ear and dorsal skin treated with MC903 on Days 1-5 and topical HSV-Ab1Fc1 (or vehicle control) on Day 7, with tissues being harvested on Day 9.

FIGS. 7A-C show mouse anti-mouse IL-4Ra antibody (Ab66Fc1) nucleic acid analyses of ear and dorsal skin in an MC903-induced atopic dermatitis model after HSV-Ab66Fc1 infection. FIG. 7A shows the levels of Ab66Fc1 DNA present in ear tissue biopsies harvested from MC903- or ethanol (EtOH) treated-animals after repeated topical application of HSV-Ab66Fc1 or vehicle control, as determined by qPCR analysis. FIG. 7B shows the levels of Ab66Fc1 DNA present in dorsal skin tissue biopsies harvested from MC903- or ethanol (EtOH) treated-animals after repeated topical application of HSV-Ab66Fc1 or vehicle control, as determined by qPCR analysis. FIG. 7C shows the levels of Ab66Fc1 transcripts present in ear tissue biopsies harvested from MC903- or ethanol (EtOH) treated-animals after repeated topical application of HSV-Ab66Fc1 or vehicle control, as determined by qRT-PCR analysis. For each condition in the qPCR and qRT-PCR analysis, data is presented for two replicates ±SEM.

FIGS. 8A-B show the effect of HSV-Ab66Fc1 or vehicle control on the development of certain ear phenotypes in an MC903-induced atopic dermatitis model. FIG. 8A shows average ear thickness on Days 1-10 of MC903- or ethanol treated-animals after repeated topical application of HSV-Ab66Fc1 or vehicle control. Asterisks indicate statistically significant differences between the MC903/Veh and MC903/Ab66 groups at each timepoint. FIG. 8B shows average ear weight on Day 10 of MC903- or ethanol treated-animals after repeated topical application of HSV-Ab66Fc1 or vehicle control. For each timepoint, data is presented for the average of four ears ±SEM. Statistics were calculated using an unpaired student's t-test: *p<0.05; **p<0.01; ***p<0.005.

### DETAILED DESCRIPTION

The following description sets forth exemplary methods, parameters, and the like. It should be recognized, however, that such a description is not intended as a limitation on the scope of the present disclosure but is instead provided as a description of exemplary embodiments.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### II. Definitions

Before describing the present disclosure in detail, it is to be understood that the present disclosure is not limited to particular compositions or biological systems, which can, of course, vary. It is also to be understood that the terminology used herein is for the purpose of describing particular embodiments only and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

As used herein, the term “and/or” may include any and all combinations of one or more of the associated listed items. For example, the term “a and/or b” may refer to “a alone”, “b alone”, “a or b”, or “a and b”; the term “a, b, and/or c” may refer to “a alone”, “b alone”, “c alone”, “a or b”, “a or c”, “b or c”, “a, b, or c”, “a and b”, “a and c” “b and c”, or “a, b, and c”; etc.

As used herein, the term “about” refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising”, “consisting”, and “consisting essentially of” aspects and embodiments.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operatively linked” or “operably linked” means that the DNA or RNA sequences being linked are contiguous.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of effecting expression of such nucleic acids. Thus, an expression vector may refer to a DNA or RNA construct, such as a plasmid, a phage, recombinant virus or other vector that, upon introduction into an appropriate host cell, results in expression of the nucleic acids. Appropriate expression vectors are well known to those of skill in the art and include those that are replicable in eukaryotic cells and those that remain episomal or those which integrate into the host cell genome.

As used herein, an “open reading frame” or “ORF” refers to a continuous stretch of nucleic acids, either DNA or RNA, that encode a protein or polypeptide. Typically, the nucleic acids comprise a translation start signal or initiation codon, such as ATG or AUG, and a termination codon.

As used herein, an “untranslated region” or “UTR” refers to untranslated nucleic acids at the 5′ and/or 3′ ends of an open reading frame. The inclusion of one or more UTRs in a polynucleotide may affect post-transcriptional regulation, mRNA stability, and/or translation of the polynucleotide.

As used herein, the term “transgene” refers to a polynucleotide that is capable of being transcribed into RNA and translated and/or expressed under appropriate conditions, after being introduced into a cell. In some aspects, it confers a desired property to a cell into which it was introduced, or otherwise leads to a desired therapeutic or diagnostic outcome.

As used herein, the terms “polypeptide,” “protein,” and “peptide” are used interchangeably and may refer to a polymer of two or more amino acids.

As used herein, the term “antibody” is used in the broadest sense, and encompasses various antibody structures, including, for example monoclonal antibodies, polyclonal antibodies, multispecific antibodies (e.g., bispecific antibodies, trispecific antibodies, etc.), and antibody fragments so long as they exhibit the desired biological activity. The term “antibody” also encompasses hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The basic 4-chain antibody unit is a heterotetrameric glycoprotein composed of two identical light chains and two identical heavy chains. The pairing of a variable heavy (V_{H}) region and variable light (V_{L}) region together forms a single antigen-binding site. For the structure and properties of the different classes of antibodies, see e.g., Basic and Clinical Immunology, 8^{th} Ed., Daniel P. Stites, Abba I Ten, and Tristram G. Parslow (eds.), Appleton & Lange, Norwalk, Conn., 1994, page 71 and Chapter 6.

The light chain from any vertebrate species can be assigned to one of two clearly distinct types, called kappa (“κ”) (see e.g., SEQ ID NO: 601 for an exemplary human kappa constant domain sequence) and lambda (“λ”) (see e.g., SEQ ID NO: 602 for an exemplary human lambda constant domain sequence), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans expressing the following subclasses: IgG1, IgG2, IgG3, IgG4, IgA1, and IgA2. The subunit structures and three-dimensional configurations of different classes of immunoglobulins are well known, and are described generally in, e.g., Abbas et al., Cellular and Molecular Immunology, 4^{th} ed. (W.B. Saunders Co., 2000).

As used herein, the terms “variable region” or “variable domain” of an antibody refer to the amino-terminal domains of the heavy or light chain of the antibody. These domains are generally the most variable parts of the antibody (relative to other antibodies of the same class) and contain the antigen binding sites.

As used herein, the term “variable” refers to the fact that certain segments of the variable domains differ extensively in sequence among antibodies. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light chain and the heavy chain variable domains. The more highly conserved portions of variable domains are called the framework regions (FR).

As used herein, the terms “hypervariable region” or “HVR” refer to the regions of an antibody variable domain that are hypervariable in sequence and/or form structurally defined loops. Generally, antibodies comprise six HVRs; three in the VH (HVR-H1, HVR-H2, and HVR-H3), and three in the VL (HVR-L1, HVR-L2, and HVR-L3). In native antibodies, HVR-H3 and HVR-L3 display the most diversity of the six HVRs, and HVR-H3 in particular is believed to play a unique role in conferring fine specificity to antibodies (see e.g., Xu et al. Immunity 13: 37-45 (2000); and Johnson and Wu, Methods in Molecular Biology 248: 1-25 (Lo, ed., Human Press, Totowa N.J., 2003)). Indeed, naturally occurring camelid antibodies consisting of a heavy chain only are functional and stable in the absence of light chain (see e.g., Hamers-Casterman et al. Nature 363: 446-448 (1993); and Sheriff et al. Nature Struct. Biol. 3: 733-736 (1996)).

A number of HVR delineations are in use and are encompassed herein. The HVRs that are EU or Kabat complementarity-determining regions (CDRs) are based on sequence variability and are the most commonly used. Chothia refers instead to the location of the structure loops (Chothia and Lesk, J. Mol. Biol. 196: 901-917 (1987)). The AbM HVRs represent a compromise between the EU or Kabat CDRs and Chothia structural loops and are used by Oxford Molecular's AbM antibody modeling software. The “contact” HVRs are based on an analysis of the available complex crystal structures.

HVRs may comprise “extended HVRs” as follows: 24-36 or 24-24 (HVR-L1), 46-56 or 50-46 (HVR-L2), and 89-97 or 89-96 (HVR-L3) in the VL, and 26-35 (HVR-H1), 50-65 or 49-65 (HVR-H2), and 93-201, 94-102, or 95-102 (HVR-H3) in the VH. The variable domain residues are numbered according to EU or Kabat et al. for each of these extended HVR definitions.

As used herein, the terms “Framework” or “FR” refer to variable domain residues other than hypervariable region (HVR) residues. The FR of a variable domain generally consists of four FR domains: FR1, FR2, FR3, and FR4. Accordingly, the HVR and FR sequences of a variable domain generally appear in the following sequence in VH (or VL): FR1-HVR-H1 (L1)-FR2-HVR-H2 (L2)-FR3-HVR-H3 (L3)-1-R4.

As used herein, the terms “full-length antibody”, “intact antibody”, or “whole antibody” are used interchangeably to refer to an antibody having a structure substantially similar to a native antibody structure or having heavy chains that contain an Fc region as defined herein. The constant domains may be native sequence constant domains (e.g., human native sequence constant domains) or amino acid sequence variants thereof. In some embodiments, the intact antibody has one or more effector functions.

As used herein, the term “Fc region” is used to define a C-terminal region of an immunoglobulin heavy chain, including the native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU or Kabat numbering system) of the Fc region may be removed, for example, during production of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Antibody “effector functions” refer to those biological activities attributable to the Fc region (a native sequence Fc region or amino acid sequence variant Fc region) of an antibody and vary with the antibody isotype.

As used herein, the term “native antibodies” refers to antibodies that are usually heterotetrameric glycoproteins of about 150,000 Daltons, composed of two identical light chains and two identical heavy chains. Each light chain is linked to a heavy chain by one covalent disulfide bond, while the number of disulfide linkages varies among the heavy chains of different immunoglobulin isotypes. Each heavy and light chain also has regularly spaced interchain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

As used herein, the term “monoclonal antibody” refers to an antibody obtained from a population of substantially homogenous antibodies, i.e., the individual antibodies comprising the population are identical except for possible naturally occurring mutations and/or post-translational modifications (e.g., isomerizations, amidations) that may be present in minor amounts. Monoclonal antibodies are highly specific, being directed against one or more antigenic sites. In some embodiments, a monoclonal antibody of the present disclosure can be multispecific (e.g., a bispecific antibody, a trispecific antibody). In contrast to polyclonal antibody preparations which typically include different antibodies directed against different determinants (epitopes), each monoclonal antibody is directed against a single determinant on the one or more antigenic sites. The modifier “monoclonal” indicates the character of the antibody as being obtained from a substantially homogenous population of antibodies and is not to be construed as requiring production of the antibody by and particular method.

As used herein, a “chimeric” antibody refers to an antibody in which a portion of the heavy and/or light chain is derived from a particular source or species, while the remainder of the heavy and/or light chain is derived from a different source or species, as well as fragments of such antibodies, so long as they exhibit the desired biological activity. As used herein, a “humanized” antibody is used as a subset of “chimeric” antibody.

As used herein, a “humanized” antibody refers to a chimeric antibody comprising amino acid residues from non-human HVRs and amino acid residues from human FRs. In some embodiments, a humanized antibody will comprise substantially all of at least one, and typically two, variable domains, in which all or substantially all of the HVRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. For further details, see e.g., Jones et al. Nature 321: 522-525 (1986); Riechmann et al. Nature 332: 323-329 (1988); Presta, Curr. Op. Stuct. Biol. 2: 593-596 (1992); Vaswani and Hamilton, Ann. Allergy, Asthma & Immunol 1: 105-115 (1998); Harris Biochem. Soc. Transactions 23: 1035-1038 (1995); Hurle and Gross, Curr. Op. Biotech. 5: 428-433 (1994); U.S. Pat. Nos. 6,982,321; and 7,087,409.

As used herein, a “human” antibody refers to an antibody which possesses an amino acid sequence which corresponds to that of an antibody produced by a human or a human cell or derived from a non-human source that utilizes human antibody repertoires or other human antibody-encoding sequences. This definition specifically excludes a humanized antibody comprising non-human antigen-binding residues.

As used herein, a “human consensus framework” refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat at al. (1991) Sequences of Proteins of Immunological Interest, Fifth Edition, NIH Publication 91-3242, Bethesda Md., vols. 1-3.

As used herein, the term “antibody fragment” refers to a molecule other than an intact antibody that comprises a portion of an intact antibody, preferably the antigen binding and/or the variable region of the intact antibody. Examples of antibody fragments may include, without limitation, Fv, Fab, Fab′, Fab′-SH, F(ab′)2, scFv, SMIP, domain antibodies, di-scFv, scFv-Fc, Nanobodies® (e.g., monovalent nanobodies, bivalent nanobodies, etc.), minibodies, diabodies, triabodies, linear antibodies, single-chain antibody molecules, and multispecific antibodies formed from antibody fragments.

Papain digestion of antibodies produces two identical antigen-binding fragments, called Fab fragments, and a residual Fc fragment (a designation reflecting the ability to crystallize readily). The Fab fragment consists of an entire light chain, along with the variable region of the heavy chain and the first constant domain (C_{H}1) of one heavy chain. Each Fab fragment is monovalent with respect to antigen binding, i.e., it has a single antigen-binding site. The Fc fragment comprises the carboxy-terminal portions of both heavy chains held together by disulfide bonds. The effector functions of antibodies are determined by sequence in the Fc region, the region which is also recognized by Fc receptors (FcR) found on certain types of cells.

Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “Fv” is the minimum antibody fragment which contains a complete antigen-recognition and binding site. This fragment consists of a dimer of one heavy and one light chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv”, also abbreviated as “sFv” or “scFv”, are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding. For a review of the scFv, see e.g., Pluckthun in The Pharmacology of Monoclonal Antibodies, vol. 113, Rosenburg and Moore (eds.), Springer-Verlag, New York, pp. 269-315 (1994), U.S. Pat. No. 6,248,516.

A “nanobody” refers to a single-domain antibody (sdAb) which is able to bind selectively to an antigen. A nanobody may comprise heavy chain variable domains and no light chain variable domains, or vice versa. A nanobody may be derived from camelids (V_{H}H antibodies) or cartilaginous fishes (V_{NAR} antibodies). Alternatively, a nanobody may be derived from splitting the dimeric variable domains from an antibody, for example, an IgG antibody, into monomers.

Diabodies are antibody fragments with two antigen-binding sites that may be bivalent or bispecific. The term “diabodies” refers to small antibody fragments prepared by constructing scFv fragments with short linkers (about 5-10 residues) between the VH and VL domains such that inter-chain but not intra-chain pairing of the V domains is achieved, thereby resulting in a bivalent fragment, i.e., a fragment having two antigen-binding sites. Bispecific diabodies are heterodimers of two “crossover” scFv fragments in which the VH and VL domains of the two antibodies are present on different polypeptide chains. Diabodies are described in greater detail in, for example, EP404097, WO93/11161, Hudson et al. (2003) Nat. Med. 9:129-134, and Hoolinger et al. PNAS USA 90: 6444-48 (1993). Triabodies and tetrabodies are also described in Hudson et al. (2003) Nat. Med. 9:129-134.

As used herein, the terms “specifically recognizes” or “specifically binds” refer to measurable and reproducible interactions, such as attraction or binding between a target and an antibody, that is determinative of the presence of the target in the presence of a heterogenous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or moiety) that specifically or preferentially binds to a first target may or may not specifically or preferentially bind to a second target. As such, “specific binding” or “preferential binding” does not necessarily require (although it can include) exclusive binding.

An “agonist” antibody or an “activating” antibody is an antibody that induces (e.g., increases) one or more activities or functions of the antigen after the antibody binds the antigen and/or that induces (e.g., increases) antigen binding to one or more ligands after the antibody binds the antigen.

A “blocking” antibody, an “antagonist” antibody, or an “inhibitory” antibody is an antibody that inhibits or reduces (e.g., decreases) antigen binding to one or more ligands after the antibody binds the antigen and/or that inhibits or reduces (e.g., decreases) one or more activities or functions of the antigen after the antibody binds the antigen. In some embodiments, blocking antibodies, antagonist antibodies, or inhibitory antibodies substantially or completely inhibits antigen binding to one or more ligands and/or substantially or completely inhibits one or more activities or functions of the antigen.

As used herein, a “subject”, “host”, or an “individual” refers to any animal classified as a mammal, including humans, domestic and farm animals, and zoo, sports, or pet animals, such as dogs, horses, cats, cows, as well as animals used in research, such as mice, rats, hamsters, rabbits, and non-human primates, etc. In some embodiments, the mammal is human.

As used herein, the terms “pharmaceutical formulation” or “pharmaceutical composition” refer to a preparation which is in such a form as to permit the biological activity of the active ingredient(s) to be effective, and which contains no additional components which are unacceptably toxic to a subject to which the composition or formulation would be administered. “Pharmaceutically acceptable” excipients (e.g., vehicles, additives) are those which can reasonably be administered to a subject to provide an effective dose of the active ingredient(s) employed.

As used herein, “cutaneous administration” or “cutaneously administering” refers to the delivery of a composition to a subject by contacting, directly or otherwise, a formulation comprising the composition to all (“systemic”) or a portion (“topical”) of the skin of a subject. The term encompasses several routes of administration including, but not limited to, topical and transdermal. Topical administration may be used as a means to deliver a composition to the epidermis or dermis of a subject, or to specific strata thereof.

As used herein, an “effective amount” is at least the minimum amount required to affect a measurable improvement or prevention of one or more symptoms of a particular disorder. An “effective amount” may vary according to factors such as the disease state, age, sex, and weight of the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications used to treat symptoms of the disease, delaying the progression of the disease, and/or prolonging survival. An effective amount can be administered in one or more administrations. For purposes of the present disclosure, an effective amount of a recombinant nucleic acid, virus, and/or pharmaceutical composition is an amount sufficient to accomplish prophylactic or therapeutic treatment either directly or indirectly. As is understood in the clinical context, an effective amount of a recombinant nucleic acid, virus, and/or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “treatment” refers to clinical intervention designed to alter the natural course of the individual or cell being treated during the course of clinical pathology. Desirable effects of treatment include decreasing the rate of disease/disorder/defect progression, ameliorating or palliating the disease/disorder/defect state, and remission or improved prognosis.

As used herein, the term “delaying progression of” a disease/disorder/defect refers to deferring, hindering, slowing, retarding, stabilizing, and/or postponing development of the disease/disorder/defect. This delay can be of varying lengths or time, depending on the history of the disease/disorder/defect and/or the individual being treated. As is evident to one of ordinary skill in the art, a sufficient or significant delay can, in effect, encompass prevention, in that the individual does not develop the disease.

### III. Recombinant Nucleic Acids

Certain aspects of the present disclosure relate to recombinant nucleic acids (e.g., isolated recombinant nucleic acids) comprising one or more (e.g., one or more, two or more, three or more, four or more, five or more, ten or more, etc.) polynucleotides encoding an antibody. The antibody may be any antibody (in any form) described herein or known in the art. In some embodiments, the antibody is a full-length antibody. In some embodiments, the antibody is an antibody fragment. In some embodiments, the antibody is an agonist antibody. In some embodiments, the antibody is an antagonist antibody.

In some embodiments, the recombinant nucleic acid is a vector. In some embodiments, the recombinant nucleic acid is a viral vector. In some embodiments, the recombinant nucleic acid is a herpes viral vector. In some embodiments, the recombinant nucleic acid is a herpes simplex virus amplicon. In some embodiments, the recombinant nucleic acid is a recombinant herpes virus genome. In some embodiments, the recombinant nucleic acid is a recombinant herpes simplex virus genome. In some embodiments, the recombinant herpes simplex virus genome is a recombinant type 1 herpes simplex virus (HSV-1) genome.

Polynucleotides Encoding Antibodies

In some embodiments, the present disclosure relates to a recombinant nucleic acid (e.g., a recombinant herpes virus genome) comprising one or more (e.g., one or more, two or more, three or more, four or more, five or more, ten or more, etc.) polynucleotides encoding an antibody. In some embodiments, at least one of the polynucleotides encodes a single-chain antibody (e.g., an scFv, an scFv-Fc, etc.). In some embodiments, at least one of the polynucleotides comprises multiple expression cassettes encoding the antibody (e.g., a first expression cassette encoding an antibody heavy chain and a second expression cassette encoding an antibody light chain, etc.). In some embodiments, at least one of the polynucleotides encodes a polycistronic mRNA encoding the antibody (e.g., a polycistronic mRNA comprising an ORF encoding an antibody heavy chain and an ORF encoding an antibody light chain separated by an IRES, etc.). In some embodiments, at least one of the polynucleotides encodes a chimeric polypeptide (e.g., a polypeptide comprising an antibody heavy chain and an antibody light chain separated by a cleavable linker, etc.). In some embodiments, the recombinant genome comprises one polynucleotide encoding an antibody. In some embodiments, the recombinant genome comprises two or more polynucleotides encoding an antibody (e.g., a first polynucleotide encoding an antibody heavy chain and a second polynucleotide encoding an antibody light chain, etc.).

In some embodiments, a first recombinant nucleic acid of the present disclosure comprises one or more polynucleotides encoding a portion of an antibody (e.g., an antibody heavy chain), and is used in conjunction with a second recombinant nucleic acid comprising one or more polynucleotides encoding a complementary portion of an antibody (e.g., an antibody light chain). In some embodiments, the first and second recombinant nucleic acids are in a single composition (e.g., contained in separate herpes simplex viruses formulated as a single pharmaceutical composition). In some embodiments, the first and second recombinant nucleic acids are in different compositions (e.g., contained in separate herpes simplex viruses formulated as two distinct pharmaceutical compositions). In some embodiments, the first recombinant nucleic acid is delivered into a target cell prior to, in conjunction with, or after delivery of the second recombinant nucleic acid into the target cell (e.g., in order to produce a single full-length antibody in one or more cells of the subject).

In some embodiments a recombinant nucleic acid of the present disclosure comprises polynucleotides encoding two or more (e.g., two or more, three or more, four or more, five or more, six or more, seven or more, eight or more, nine or more, ten or more, etc.) antibodies. In some embodiments, the two or more antibodies are the same. In some embodiments, the two or more antibodies are different.

Antibodies

Antibodies encoded by one or more of the polynucleotides of the present disclosure may be from any suitable species known in the art, including, for example, human antibodies, mouse antibodies, rat antibodies, rabbit antibodies, camelid antibodies, chicken antibodies, donkey antibodies, feline antibodies, goat antibodies, sheep antibodies, horse antibodies, hamster antibodies, guinea pig antibodies, shark antibodies, and any chimeric antibodies thereof. In some embodiments, the antibody is a human antibody. In some embodiments, the antibody is a mouse antibody. In some embodiments the antibody is a chimeric antibody (e.g., a human-mouse chimeric antibody). In some embodiments, the antibody is a humanized antibody.

Antibodies encoded by one or more of the polynucleotides of the present disclosure may be of any suitable isotype known in the art, including, for example, IgA, IgD, IgE, IgG, IgM, and any combinations thereof. In some embodiments, the antibody is an IgG antibody. In some embodiments, the IgG antibody is an IgG1 antibody (see e.g., SEQ ID NOS: 596 or 597 for exemplary human IgG1 constant region sequences), an IgG2 antibody (see e.g., SEQ ID NO: 598 for an exemplary human IgG2 constant region sequence), an IgG3 antibody (see e.g., SEQ ID NO: 599 for an exemplary IgG3 constant region sequence), an IgG4 antibody (see e.g., SEQ ID NO: 600 for an exemplary human IgG4 constant region sequence), and any chimeric IgG antibodies thereof. In some embodiments, the IgG antibody is an IgG1 antibody.

In some embodiments, an antibody encoded by one or more polynucleotides of the present disclosure is an antibody fragment. Any type or form of antibody fragment known in the art may be encoded by a polynucleotide of the present disclosure including, for example, a Fab fragment, a Fab′ fragment, a Fab′-SH fragment, a F(ab′)2 fragment, an Fv fragment, an scFv fragment, an scFv-Fc fragment, as well as any other type or form of antibody fragment described herein or known in the art. In some embodiment, the antibody fragment is a Fab fragment. In some embodiments, the antibody fragment is an scFv. In some embodiments, the antibody fragment is an scFv-Fc. For a review of certain antibody fragments, see e.g., Hudson et al. (2003) Nat. Med. 9:129-134, Pluckthün The Pharmacology of Monoclonal Antibodies vol. 113, Rosenburg and Moore eds. (Springer-Verlag, New York) pp. 269-315 (1994), WO93/16185, U.S. Pat. Nos. 5,571,894, 5,587,458, and 5,869,046.

In some embodiments, an antibody encoded by one or more polynucleotides of the present disclosure is a chimeric antibody. Certain chimeric antibodies are described, e.g., in U.S. Pat. No. 4,816,567, and Morrison et al. (1984) PNAS USA 81:6851-6855. In some embodiments, a chimeric antibody comprises a non-human variable region (e.g., a variable region derived from a mouse, rat, hamster, rabbit, non-human primate, etc.) and a human constant region. In some embodiments, a chimeric antibody is a “class switched” antibody in which the class or subclass has been changed from that of the parental antibody. Chimeric antibodies include antigen-binding fragments thereof. In some embodiments, the chimeric antibody is a mouse-human chimeric antibody.

In some embodiments, an antibody encoded by one or more polynucleotides of the present disclosure is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity and/or affinity.

Humanized antibodies, and methods of making the same, are reviewed, e.g., in Almagro and Fransson (2008) Front. Biosci. 13: 1619-1633, Riechmann et al. (1988) Nature 332: 323-329, Queen et al. (1989) PNAS USA 86: 10029-10033, U.S. Pat. Nos. 5,821,337, 7,527,791, 6,982,321, 7,087,409, Kashmiri et al. (2005) Methods 36: 25-34 (describing specificity determining region (SDR) grafting), Padlam (1991) Mol Immunol 28: 489-498 (describing “resurfacing”), Dall'Acqua et al. (2005) Methods 36: 43-60 (describing “FR shuffling”), Osbourn et al. (2005) Methods 36: 61-68, and Klimka et al. (2000) Br J Cancer 83: 252-260 (describing the “guided selection” approach to FR shuffling).

Human framework regions that may be used for humanization include, for example, framework regions selected using the “best-fit” method (see e.g., Sims et al. (1993) J Immunol 151: 2296), framework regions derived from the consensus sequence of human antibodies of a particular subgroup of light or heavy chain variable regions (see e.g., Carter et al. (1992) PNAS USA 89: 4285; see also Presta et al. (1993) J Immunol 151: 2623), human mature (somatically mutated) framework regions or human germline framework regions (see e.g., Almagro and Fransson (2008) Front Biosci 13: 1619-1633), and framework regions derived from screening FR libraries (see e.g., Baca et al. (1997) J Biol Chem 272: 10678-10684; see also Rosok et al. (1996) J Biol Chem 271: 22611-22618).

In some embodiments, an antibody encoded by one or more polynucleotides of the present disclosure is a human antibody. Human antibodies are generally described in van Dijk and van de Winkel (2001) Curr Opin Pharmacol 5: 368-74 and Lonberg (2008) Curr Opin Immunol 20: 450-459. Certain details regarding human antibodies can be found in, e.g., Hoogenboom and Winter, J. Mol. Biol. 227: 381 (1991); Marks et al. J. Mol. Biol. 222: 581 (1991); Cole et al. Monoclonal Antibodies and Cancer Therapy, p. 77 (1985); Boerner et al. J. Immunol. 147(1): 86-95 (1991); van Dijk and van de Winkel, Curr. Opin. Pharmacol. 5: 368-74 (2001); U.S. Pat. Nos. 6,075,181; 6,150,584; and Li et al. PNAS USA 103: 3557-3562 (2006).

In some embodiments, an antibody encoded by one or more polynucleotides of the present disclosure is a multispecific antibody (e.g., a bispecific antibody, a trispecific antibody, etc.). Techniques for making multispecific antibodies include, for example, recombinant co-expression of two immunoglobulin heavy chain-light chain pairs having different specificities (see e.g., Milstein and Cuello, Nature 305: 537 (1983), WO 93/08829, Traunecker et al. EMBO J. 10: 3655 (1991), and “knob-in-hole” engineering (e.g., as described in in U.S. Pat. No. 5,731,168)). Multispecific antibodies may also be made by engineering electrostatic steering effects for making antibody Fc-heterodimeric molecules (see e.g., WO2009/089004); cross-linking two or more antibodies or fragments (see e.g., U.S. Pat. No. 4,676,980, Brennan et al. Science, 229: 81 (1985)); using leucine zippers to produce bispecific antibodies (see e.g., Kostelny et al. J Immunol, 148(5): 1547-53 (1992)); using “diabody” technology for making bispecific antibody fragments (see e.g., Hollinger et al. PNAS USA 90:6444-8 (1993); using single-chain Fv dimers (see e.g., Gruber et al. J Immunol. 152:5368 (1994)); using dual acting Fabs (see e.g., US2008/0069820); and preparing trispecific or trivalent antibodies (see e.g., Tutt et al. J Immunol 147:60 (1991), WO2017/074878).

Antibodies (or antigen-binding fragments thereof) encoded by one or more polynucleotides of the present disclosure may contain: 1) an HVR-H1, HVR-H2, HVR-H3, HVR-L1, HVR-L2, and HVR-L3 of any antibody described herein or known in the art; 2) the heavy chain variable region and/or light chain variable region of any antibody described herein or known in the art; and/or 3) the full-length heavy chain and/or full-length light chain of any antibody described herein or known in the art. Examples of suitable antibodies that may be encoded by the polynucleotides of the present disclosure include, for example, abagovomab, abciximab (V_{H}—SEQ ID NO: 617; V_{L}—SEQ ID NO: 869), abituzumab (V_{H}—SEQ ID NO: 618; V_{L}—SEQ ID NO: 870), abrezekimab, abrilumab (V_{H}—SEQ ID NO: 619; V_{L}—SEQ ID NO: 871), actoxumab (V_{H}—SEQ ID NO: 620; V_{L}—SEQ ID NO: 872), adalimumab (V_{H}-SEQ ID NO: 355; V_{L}—SEQ ID NO: 420), adecatumumab, aducanumab (V_{H}—SEQ ID NO: 621; V_{L}—SEQ ID NO: 873), afasevikumab (V_{H}—SEQ ID NO: 622; V_{L}—SEQ ID NO: 874), afelimomab, afutuzumab, alacizumab (V_{H}—SEQ ID NO: 629; V_{L}—SEQ ID NO: 881), alemtuzumab (V_{H}—SEQ ID NO: 382; V_{L}—SEQ ID NO: 447), alirocumab (V_{H}—SEQ ID NO: 392; V_{L}—SEQ ID NO: 457), altumomab, amatuximab (V_{H}—SEQ ID NO: 623; V_{L}—SEQ ID NO: 875), anatumomab, andecaliximab (V_{H}—SEQ ID NO: 624; V_{L}—SEQ ID NO: 876), anetumab (V_{H}—SEQ ID NO: 625; V_{L}—SEQ ID NO: 877), anifrolumab (V_{H}—SEQ ID NO: 387; V_{L}—SEQ ID NO: 452), anrukinzumab (V_{H}—SEQ ID NO: 626; V_{L}—SEQ ID NO: 878), apolizumab, aprutumab (V_{H}—SEQ ID NO: 627; V_{L}—SEQ ID NO: 879), arcitumomab (V_{H}—SEQ ID NO: 628; V_{L}—SEQ ID NO: 880), ascrinvacumab, aselizumab, atezolizumab (V_{H}—SEQ ID NO: 374; V_{L}—SEQ ID NO: 439), atinumab, atlizumab, atorolimumab, avelumab (V_{H}—SEQ ID NO: 630; V_{L}—SEQ ID NO: 882), azintuxizumab (V_{H}—SEQ ID NO: 631; V_{L}—SEQ ID NO: 883), bapineuzumab (V_{H}—SEQ ID NO: 632; V_{L}—SEQ ID NO: 884), basiliximab (V_{H}—SEQ ID NO: 389; V_{L}—SEQ ID NO: 454), bavituximab (V_{H}—SEQ ID NO: 633; V_{L}—SEQ ID NO: 885), bectumomab, begelomab (V_{H}—SEQ ID NO: 634; V_{L}—SEQ ID NO: 886), belantamab (V_{H}—SEQ ID NO: 635; V_{L}—SEQ ID NO: 887), belimumab (V_{H}—SEQ ID NO: 388; V_{L}—SEQ ID NO: 453), bemarituzumab, belimumab, bemaritzumab (V_{H}—SEQ ID NO: 636; V_{L}—SEQ ID NO: 888), benralizumab (V_{H}—SEQ ID NO: 637; V_{L}—SEQ ID NO: 889), berlimatoxumab (V_{H}—SEQ ID NO: 638; V_{L}—SEQ ID NO: 890), bersanlimab (V_{H}—SEQ ID NO: 639; V_{L}—SEQ ID NO: 891), bertilimumab, besilesomab, bevacizumab (V_{H}—SEQ ID NO: 357; V_{L}—SEQ ID NO: 422), bezlotoxumab (V_{H}—SEQ ID NO: 640; V_{L}—SEQ ID NO: 892), biciromab, bimagrumab (V_{H}—SEQ ID NO: 415; V_{L}—SEQ ID NO:478), bimekizumab (V_{H}—SEQ ID NO: 641; V_{L}—SEQ ID NO: 893), birtamimab (V_{H}—SEQ ID NO: 642; V_{L}—SEQ ID NO: 894), bivatuzumab, bleselumab (V_{H}—SEQ ID NO: 643; V_{L}—SEQ ID NO: 895), blinatumomab (V_{H}—SEQ ID NO: 644; V_{L}—SEQ ID NO: 896), blontuvetmab (V_{H}—SEQ ID NO: 645; V_{L}—SEQ ID NO: 897), blosozumab (V_{H}—SEQ ID NO: 646; V_{L}—SEQ ID NO: 898), bococizumab (V_{H}—SEQ ID NO: 647; V_{L}—SEQ ID NO: 899), brazikumab (V_{H}—SEQ ID NO: 648; V_{L}—SEQ ID NO: 900), brentuximab (V_{H}—SEQ ID NO: 649; V_{L}—SEQ ID NO: 901), briakinumab (V_{H}—SEQ ID NO: 650; V_{L}—SEQ ID NO: 902), brodalumab (V_{H}—SEQ ID NO: 361; V_{L}—SEQ ID NO: 426), brolucizumab (V_{H}—SEQ ID NO: 651; V_{L}—SEQ ID NO: 903), brontictuzumab (V_{H}—SEQ ID NO: 652; V_{L}—SEQ ID NO: 904), burosumab (V_{H}—SEQ ID NO: 653; V_{L}—SEQ ID NO: 905), cabiralizumab (V_{H}—SEQ ID NO: 654; V_{L}—SEQ ID NO: 906), camidanlumab (V_{H}—SEQ ID NO: 655; V_{L}—SEQ ID NO: 907), camrelizumab (V_{H}—SEQ ID NO: 656; V_{L}—SEQ ID NO: 908), canakinumab (V_{H}—SEQ ID NO: 378; V_{L}—SEQ ID NO: 443), cantuzumab (V_{H}—SEQ ID NO: 657; V_{L}—SEQ ID NO: 909), caplacizumab, capromab, carlumab (V_{H}—SEQ ID NO: 658; V_{L}-SEQ ID NO: 910), carotuximab (V_{H}—SEQ ID NO: 659; V_{L}—SEQ ID NO: 911), catumaxomab, cedelizumab, cemiplimab (V_{H}—SEQ ID NO: 394; V_{L}—SEQ ID NO: 459), cergutuzumab (V_{H}—SEQ ID NO: 660; V_{L}—SEQ ID NO: 912), certolizumab (V_{H}—SEQ ID NO: 370; V_{L}—SEQ ID NO: 435), cetrelimab (V_{H}—SEQ ID NO: 661; V_{L}—SEQ ID NO: 913), cetuximab (V_{H}—SEQ ID NO: 662; V_{L}—SEQ ID NO: 914), cibisatamab (bispecific: V_{H1}—SEQ ID NO: 660; V_{L1}—SEQ ID NO: 915; V_{H2}—SEQ ID NO: 660; V_{L2}—SEQ ID NO: 912), citatuzumab (V_{H}—SEQ ID NO: 663; V_{L}—SEQ ID NO: 916), cixutumumab (V_{H}—SEQ ID NO: 664; V_{L}—SEQ ID NO: 917), clazakizumab (V_{H}—SEQ ID NO: 665; V_{L}—SEQ ID NO: 918), clenoliximab, clivatuzumab (V_{H}—SEQ ID NO: 666; V_{L}—SEQ ID NO: 919), codrituzumab (V_{H}—SEQ ID NO: 667; V_{L}—SEQ ID NO: 920), cofetuzumab (V_{H}—SEQ ID NO: 668; V_{L}—SEQ ID NO: 921), coltuximab (V_{H}—SEQ ID NO: 669; V_{L}—SEQ ID NO: 922), conatumumab (V_{H}—SEQ ID NO: 670; V_{L}—SEQ ID NO: 923), concizumab (V_{H}—SEQ ID NO: 671; V_{L}—SEQ ID NO: 924), cosfroviximab (V_{H}—SEQ ID NO: 672; V_{L}—SEQ ID NO: 925), crenezumab (V_{H}—SEQ ID NO: 673; V_{L}—SEQ ID NO: 926), crizanlizumab (V_{H}—SEQ ID NO: 674; V_{L}—SEQ ID NO: 927), crotedumab (V_{H}—SEQ ID NO: 675; V_{L}—SEQ ID NO: 928), cusatuzumab (V_{H}—SEQ ID NO: 676; V_{L}—SEQ ID NO: 929), dacetuzumab (V_{H}—SEQ ID NO: 677; V_{L}—SEQ ID NO: 930), daclizumab (V_{H}—SEQ ID NO: 390; V_{L}—SEQ ID NO: 455), dalotuzumab (V_{H}—SEQ ID NO: 678; V_{L}—SEQ ID NO: 931), dapirolizumab (V_{H}—SEQ ID NO: 679; V_{L}—SEQ ID NO: 932), daratumumab (V_{H}—SEQ ID NO: 680; V_{L}—SEQ ID NO: 933), dectrekumab (V_{H}—SEQ ID NO: 681; V_{L}—SEQ ID NO: 934), demcizumab (V_{H}—SEQ ID NO: 682; V_{L}—SEQ ID NO: 935), denintuzumab (V_{H}—SEQ ID NO: 683; V_{L}—SEQ ID NO: 936), denosumab (V_{H}—SEQ ID NO: 684; V_{L}—SEQ ID NO: 937), depatuxizumab (V_{H}—SEQ ID NO: 685; V_{L}—SEQ ID NO: 938), derlotuximab (V_{H}—SEQ ID NO: 686; V_{L}—SEQ ID NO: 939), detumomab, dezamizumab (V_{H}—SEQ ID NO: 687; V_{L}—SEQ ID NO: 940), dinutuximab (V_{H}—SEQ ID NO: 688; V_{L}—SEQ ID NO: 941), diridavumab (V_{H}—SEQ ID NO: 689; V_{L}—SEQ ID NO: 942), domagrozumab (V_{H}—SEQ ID NO: 690; V_{L}—SEQ ID NO: 943), dorlimomab, drozitumab (V_{H}—SEQ ID NO: 691; V_{L}—SEQ ID NO: 944), duligotuzumab (V_{H}—SEQ ID NO: 692; V_{L}—SEQ ID NO: 945), dupilumab (V_{H}—SEQ ID NO: 391; V_{L}—SEQ ID NO: 456), durvalumab (V_{H}—SEQ ID NO: 375; V_{L}—SEQ ID NO: 440), dusigitumab (V_{H}—SEQ ID NO: 693; V_{L}—SEQ ID NO: 946), duvortuxizumab (bispecific: V_{H1}—SEQ ID NO: 694; V_{L1}—SEQ ID NO: 947; V_{H2}—SEQ ID NO: 695; V_{L2}—SEQ ID NO: 948), ecromeximab, eculizumab (V_{H}—SEQ ID NO: 385; V_{L}—SEQ ID NO: 450), edobacomab, edrecolomab, efalizumab (V_{H}—SEQ ID NO: 696; V_{L}—SEQ ID NO: 949), efungumab, eldelumab (V_{H}—SEQ ID NO: 697; V_{L}—SEQ ID NO: 950), elezanumab (V_{H}—SEQ ID NO: 698; V_{L}—SEQ ID NO: 951), elgemtumab (V_{H}—SEQ ID NO: 699; V_{L}—SEQ ID NO: 952), elotuzumab (V_{H}—SEQ ID NO: 700; V_{L}—SEQ ID NO: 953), elsilimomab, emactuzumab (V_{H}—SEQ ID NO: 701; V_{L}—SEQ ID NO: 954), emapalumab (V_{H}—SEQ ID NO: 702; V_{L}—SEQ ID NO: 955), emibetuzumab (V_{H}—SEQ ID NO: 703; V_{L}—SEQ ID NO: 956), emicizumab (bispecific: V_{H1}—SEQ ID NO: 704; V_{L1}—SEQ ID NO: 957; V_{H2}—SEQ ID NO: 705; V_{L2}—SEQ ID NO: 957), enapotamab (V_{H}—SEQ ID NO: 706; V_{L}-SEQ ID NO: 958), enavatuzumab (V_{H}—SEQ ID NO: 707; V_{L}—SEQ ID NO: 959), enfortumab (V_{H}—SEQ ID NO: 708; V_{L}—SEQ ID NO: 960), enlimomab, enoblituzumab (V_{H}—SEQ ID NO: 371; V_{L}—SEQ ID NO: 436), enokizumab (V_{H}—SEQ ID NO: 709; V_{L}—SEQ ID NO: 961), enoticumab (V_{H}—SEQ ID NO: 710; V_{L}—SEQ ID NO: 962), ensituximab (V_{H}—SEQ ID NO: 711; V_{L}—SEQ ID NO: 963), epitumomab, epratuzumab (V_{H}—SEQ ID NO: 712; V_{L}—SEQ ID NO: 964), eptinezumab (V_{H}—SEQ ID NO: 713; V_{L}—SEQ ID NO: 965), erenumab (V_{H}—SEQ ID NO: 414; V_{L}—SEQ ID NO: 477), erlizumab, ertumaxomab, etaracizumab (V_{H}—SEQ ID NO: 714; V_{L}—SEQ ID NO: 966), etigilimab (V_{H}—SEQ ID NO: 715; V_{L}—SEQ ID NO: 967), etrolizumab (V_{H}—SEQ ID NO: 716; V_{L}—SEQ ID NO: 968), evinacumab (V_{H}—SEQ ID NO: 406; V_{L}—SEQ ID NO: 470), evolocumab (V_{H}—SEQ ID NO: 717; V_{L}—SEQ ID NO: 969), exbivirumab, fanolesomab, faralimomab, faricimab, farletuzumab (V_{H}—SEQ ID NO: 718; V_{L}—SEQ ID NO: 970), fasinumab (V_{H}—SEQ ID NO: 407; V_{L}—SEQ ID NO: 471), felvizumab fezakinumab (V_{H}—SEQ ID NO: 719; V_{L}—SEQ ID NO: 971), fibatuzumab, ficlatuzumab (V_{H}—SEQ ID NO: 720; V_{L}—SEQ ID NO: 972), figitumumab (V_{H}—SEQ ID NO: 721; V_{L}—SEQ ID NO: 973), firivumab (V_{H}—SEQ ID NO: 722; V_{L}—SEQ ID NO: 974), flanvotumab (V_{H}—SEQ ID NO: 723; V_{L}—SEQ ID NO: 975), fletikumab (V_{H}—SEQ ID NO: 724; V_{L}—SEQ ID NO: 976), flotetuzumab, fontolizumab, foralumab (V_{H}—SEQ ID NO: 725; V_{L}—SEQ ID NO: 977), foravirumab (V_{H}—SEQ ID NO: 726; V_{L}—SEQ ID NO: 978), fremanezumab, fresolimumab (SEQ ID NO: 396; V_{L}—SEQ ID NO: 461), frunevetmab, fulranumab (V_{H}—SEQ ID NO: 727; V_{L}—SEQ ID NO: 976), futuximab (V_{H}—SEQ ID NO: 728; V_{L}—SEQ ID NO: 979), galcanezumab (V_{H}—SEQ ID NO: 729; V_{L}—SEQ ID NO: 980), galiximab (V_{H}—SEQ ID NO: 730; V_{L}—SEQ ID NO: 981), gancotamab, ganitumab (V_{H}—SEQ ID NO: 731; V_{L}—SEQ ID NO: 982), gantenerumab (V_{H}—SEQ ID NO: 732; V_{L}—SEQ ID NO: 983), gatipotuzumab, gavilimomab, gedivumab, gemtuzumab (V_{H}—SEQ ID NO: 733; V_{L}—SEQ ID NO: 984), gevokizumab (V_{H}—SEQ ID NO: 734; V_{L}—SEQ ID NO: 985), gilvetmab, gimsilumab, girentuximab (V_{H}—SEQ ID NO: 735; V_{L}—SEQ ID NO: 986), glembatumumab (V_{H}—SEQ ID NO: 736; V_{L}—SEQ ID NO: 987), golimumab (V_{H}—SEQ ID NO: 369; V_{L}—SEQ ID NO: 434), gomiliximab, gosuranemab, guselkumab (V_{H}—SEQ ID NO: 362; V_{L}—SEQ ID NO: 427), ianalumab, ibalizumab (V_{H}—SEQ ID NO: 737; V_{L}—SEQ ID NO: 988), ibritumomab, icrucumab (V_{H}—SEQ ID NO: 738; V_{L}—SEQ ID NO: 989), idarucizumab (V_{H}—SEQ ID NO: 739; V_{L}—SEQ ID NO: 990), ifabotuzumab, igovomab, iladatuzumab, imalumab (V_{H}—SEQ ID NO: 740; V_{L}—SEQ ID NO: 991), imaprelimab, imciromab, imgatuzumab (V_{H}—SEQ ID NO: 741; V_{L}—SEQ ID NO: 992), inclacumab (V_{H}—SEQ ID NO: 742; V_{L}—SEQ ID NO: 993), indatuximab (V_{H}—SEQ ID NO: 743; V_{L}—SEQ ID NO: 994), indusatumab (V_{H}—SEQ ID NO: 744; V_{L}—SEQ ID NO: 995), inebilizumab, inflectra, infliximab (V_{H}—SEQ ID NO: 365; V_{L}—SEQ ID NO: 430), intetumumab (V_{H}—SEQ ID NO: 745; V_{L}—SEQ ID NO: 996), inolimomab, inotuzumab (V_{H}—SEQ ID NO: 746; V_{L}—SEQ ID NO: 997), ipilimumab (V_{H}—SEQ ID NO: 372; V_{L}—SEQ ID NO: 437), iratumumab, isatuximab (V_{H}—SEQ ID NO: 393; V_{L}—SEQ ID NO: 458), iscalimab, istiratumab, itolizumab (V_{H}—SEQ ID NO: 747; V_{L}—SEQ ID NO: 998), ixekizumab (V_{H}—SEQ ID NO: 360; V_{L}—SEQ ID NO: 425), keliximab, labetuzumab (V_{H}—SEQ ID NO: 748; V_{L}—SEQ ID NO: 999), lacnotuzumab, ladiratuzumab, lampalizumab (V_{H}—SEQ ID NO: 749; V_{L}—SEQ ID NO: 1000), lanadelumab, landogrozumab (V_{H}—SEQ ID NO: 750; V_{L}—SEQ ID NO: 1001), laprituximab, larcaviximab, lebrikizumab (V_{H}—SEQ ID NO: 751; V_{L}—SEQ ID NO: 1002), lemalesomab, lendalizumab, lenvervimab, lenzilumab (V_{H}—SEQ ID NO: 752; V_{L}—SEQ ID NO: 1003), lerdelimumab, leronlimab, lesofavumab, letolizumab, lexatumumab, libivirumab, lifastuzumab (V_{H}—SEQ ID NO: 753; V_{L}—SEQ ID NO: 1004), ligelizumab (V_{H}—SEQ ID NO: 417; V_{L}—SEQ ID NO: 480), loncastuximab, losatuxizumab, lilotomab (V_{H}—SEQ ID NO: 754; V_{L}—SEQ ID NO: 1005), lintuzumab (V_{H}—SEQ ID NO: 755; V_{L}—SEQ ID NO: 1006), lirilumab (V_{H}—SEQ ID NO: 756; V_{L}—SEQ ID NO: 1007), lodelcizumab (V_{H}—SEQ ID NO: 757; V_{L}—SEQ ID NO: 1008), lokivetmab (V_{H}—SEQ ID NO: 758; V_{L}—SEQ ID NO: 1009), lorvotuzumab (V_{H}—SEQ ID NO: 759; V_{L}—SEQ ID NO: 1010), lucatumumab, lulizumab, lumiliximab (V_{H}—SEQ ID NO: 760; V_{L}—SEQ ID NO: 1011), lumretuzumab (V_{H}—SEQ ID NO: 761; V_{L}—SEQ ID NO: 1012), lupartumab, lutikizumab, mapatumumab, margetuximab (V_{H}—SEQ ID NO: 762; V_{L}—SEQ ID NO: 1013), marstacimab, maslimomab, mavrilimumab (V_{H}—SEQ ID NO: 366; V_{L}—SEQ ID NO: 431), matuzumab (V_{H}—SEQ ID NO: 763; V_{L}—SEQ ID NO: 1014), mepolizumab (V_{H}—SEQ ID NO: 764; V_{L}—SEQ ID NO: 1015), metelimumab, milatuzumab (V_{H}—SEQ ID NO: 765; V_{L}—SEQ ID NO: 1016), minretumomab, mirikizumab, mirvetuximab (V_{H}—SEQ ID NO: 766; V_{L}—SEQ ID NO: 1017), mitumomab, modotuximab (V_{H}—SEQ ID NO: 767; V_{L}—SEQ ID NO: 1018), mogamulizumab (V_{H}—SEQ ID NO: 768; V_{L}—SEQ ID NO: 1019), monalizumab (V_{H}—SEQ ID NO: 769; V_{L}—SEQ ID NO: 1020), morolimumab, mosunetuzumab, motavizumab (V_{H}—SEQ ID NO: 770; V_{L}—SEQ ID NO: 1021), moxetumomab, muromonab (V_{H}—SEQ ID NO: 771; V_{L}—SEQ ID NO: 1022), nacolomab, namilumab (V_{H}—SEQ ID NO: 772; V_{L}—SEQ ID NO: 1023), naptumomab, naratuximab, narnatumab (V_{H}—SEQ ID NO: 773; V_{L}—SEQ ID NO: 1024), natalizumab (V_{H}—SEQ ID NO: 384; V_{L}—SEQ ID NO: 449), navicixizumab, navivumab (V_{H}—SEQ ID NO: 774; V_{L}—SEQ ID NO: 1025), naxitamab, nebacumab, necitumumab (V_{H}—SEQ ID NO: 775; V_{L}—SEQ ID NO: 1026), nemolizumab (V_{H}—SEQ ID NO: 776; V_{L}—SEQ ID NO: 1027), nerelimomab, nesvacumab (V_{H}—SEQ ID NO: 777; V_{L}—SEQ ID NO: 1028), netakimab, nimotuzumab, nirsevimab, nivolumab (V_{H}—SEQ ID NO: 376; V_{L}—SEQ ID NO: 441), nofetumomab, obiltoxaximab (V_{H}—SEQ ID NO: 778; V_{L}—SEQ ID NO: 1029), obinutuzumab (V_{H}—SEQ ID NO: 779; V_{L}—SEQ ID NO: 1030), ocaratuzumab (V_{H}—SEQ ID NO: 780; V_{L}—SEQ ID NO: 1031), ocrelizumab (V_{H}—SEQ ID NO: 379; V_{L}—SEQ ID NO: 444), odulimomab, ofatumumab (V_{H}—SEQ ID NO: 380; V_{L}—SEQ ID NO: 445), olaratumab (V_{H}—SEQ ID NO: 781; V_{L}—SEQ ID NO: 1032), oleclumab, olendalizumab, olokizumab (V_{H}—SEQ ID NO: 782; V_{L}—SEQ ID NO: 1033), omalizumab (V_{H}—SEQ ID NO: 418; V_{L}—SEQ ID NO: 481), onartuzumab (V_{H}—SEQ ID NO: 783; V_{L}—SEQ ID NO: 1034), ontuxizumab (V_{H}—SEQ ID NO: 37; V_{L}—SEQ ID NO: 438), onvatilimab, opicinumab (V_{H}—SEQ ID NO: 383; V_{L}—SEQ ID NO: 448), oportuzumab, oregovomab, orticumab (V_{H}—SEQ ID NO: 784; V_{L}—SEQ ID NO: 1035), otelixizumab (V_{H}—SEQ ID NO: 785; V_{L}—SEQ ID NO: 1036), otilimab, otlertuzumab (V_{H}—SEQ ID NO: 786; V_{L}—SEQ ID NO: 1037), oxelumab (V_{H}—SEQ ID NO: 787; V_{L}—SEQ ID NO: 1038), ozanezumab (V_{H}—SEQ ID NO: 788; V_{L}—SEQ ID NO: 1039), ozoralizumab, pagibaximab, palivizumab (V_{H}—SEQ ID NO: 789; V_{L}—SEQ ID NO: 1040), pamrevlumab (V_{H}—SEQ ID NO: 790; V_{L}—SEQ ID NO: 1041), panitumumab (V_{H}—SEQ ID NO: 791; V_{L}—SEQ ID NO: 1042), pankomab, panobacumab (V_{H}—SEQ ID NO: 792; V_{L}—SEQ ID NO: 1043), parsatuzumab (V_{H}—SEQ ID NO: 793; V_{L}—SEQ ID NO: 1044), pascolizumab, pasotuxizumab, pateclizumab (V_{H}—SEQ ID NO: 794; V_{L}—SEQ ID NO: 1045), patritumab (V_{H}—SEQ ID NO: 795; V_{L}—SEQ ID NO: 1046), pembrolizumab (V_{H}—SEQ ID NO: 377; V_{L}—SEQ ID NO: 442), pemtumomab, perakizumab (V_{H}—SEQ ID NO: 796; V_{L}—SEQ ID NO: 1047), pertuzumab (V_{H}—SEQ ID NO: 797; V_{L}—SEQ ID NO: 1048), pexelizumab, pidilizumab (V_{H}—SEQ ID NO: 798; V_{L}—SEQ ID NO: 1049), pinatuzumab (V_{H}—SEQ ID NO: 799; V_{L}—SEQ ID NO: 1050), pintumomab, placulumab, plozalizumab (V_{H}—SEQ ID NO: 800; V_{L}—SEQ ID NO: 1051), pogalizumab, polatuzumab (V_{H}—SEQ ID NO: 801; V_{L}—SEQ ID NO: 1052), ponezumab (V_{H}—SEQ ID NO: 802; V_{L}—SEQ ID NO: 1053), porgaviximab, prasinezumab, prezalizumab, priliximab, pritoxaximab (V_{H}—SEQ ID NO: 803; V_{L}—SEQ ID NO: 1054), pritumumab, quilizumab (V_{H}—SEQ ID NO: 804; V_{L}—SEQ ID NO: 1055), racotumomab (V_{H}—SEQ ID NO: 805; V_{L}—SEQ ID NO: 1056), radretumab (V_{H}—SEQ ID NO: 806; V_{L}—SEQ ID NO: 1057), rafivirumab (V_{H}—SEQ ID NO: 807; V_{L}—SEQ ID NO: 1058), ralpancizumab (V_{H}—SEQ ID NO: 808; V_{L}—SEQ ID NO: 1059), ramucirumab (V_{H}—SEQ ID NO: 809; V_{L}—SEQ ID NO: 1060), ranevetmab, ranibizumab (V_{H}—SEQ ID NO: 416; V_{L}—SEQ ID NO: 479), raxibacumab, ravagalimab, ravulizumab, refanezumab (V_{H}—SEQ ID NO: 810; V_{L}—SEQ ID NO: 1061), regavirumab, remtolumab, reslizumab (V_{H}—SEQ ID NO: 811; V_{L}—SEQ ID NO: 1062), rilotumumab (V_{H}—SEQ ID NO: 812; V_{L}—SEQ ID NO: 1063), rinucumab (V_{H}—SEQ ID NO: 813; V_{L}—SEQ ID NO: 1064), risankizumab (V_{H}—SEQ ID NO: 363; V_{L}—SEQ ID NO: 428), rituximab (V_{H}—SEQ ID NO: 356; V_{L}—SEQ ID NO: 421), rivabazumab (V_{H}—SEQ ID NO: 814; V_{L}—SEQ ID NO: 1065), robatumumab (V_{H}—SEQ ID NO: 815; V_{L}—SEQ ID NO: 1066), roledumab (V_{H}—SEQ ID NO: 816; V_{L}—SEQ ID NO: 1067), romilkimab, romosozumab (V_{H}—SEQ ID NO: 817; V_{L}—SEQ ID NO: 1068), rontalizumab (V_{H}—SEQ ID NO: 818; V_{L}—SEQ ID NO: 1069), rosmantuzumab, rovalpituzumab (V_{H}—SEQ ID NO: 819; V_{L}—SEQ ID NO: 1070), rovelizumab, rozanolixizumab, ruplizumab, sacituzumab (V_{H}—SEQ ID NO: 820; V_{L}—SEQ ID NO: 1071), samalizumab (V_{H}—SEQ ID NO: 821; V_{L}—SEQ ID NO: 1072), samrotamab, sapelizumab, sarilumab (V_{H}—SEQ ID NO: 368; V_{L}—SEQ ID NO: 433), satralizumab (V_{H}—SEQ ID NO: 386; V_{L}—SEQ ID NO: 451), satumomab (V_{H}—SEQ ID NO: 822; V_{L}—SEQ ID NO: 1073), secukinumab (V_{H}—SEQ ID NO: 359; V_{L}—SEQ ID NO: 424), selicrelumab, seribantumab (V_{H}—SEQ ID NO: 823; V_{L}—SEQ ID NO: 1074), setoxaximab (V_{H}—SEQ ID NO: 824; V_{L}—SEQ ID NO: 1075), setrusumab, sevirumab, sibrotuzumab, sifalimumab (V_{H}—SEQ ID NO: 825; V_{L}—SEQ ID NO: 1076), siltuximab (V_{H}—SEQ ID NO: 826; V_{L}—SEQ ID NO: 1077), simtuzumab (V_{H}—SEQ ID NO: 827; V_{L}—SEQ ID NO: 1078), siplizumab, sirtratumab, sirukumab (V_{H}—SEQ ID NO: 828; V_{L}—SEQ ID NO: 1079), sofituzumab (V_{H}—SEQ ID NO: 829; V_{L}—SEQ ID NO: 1080), solanezumab (V_{H}—SEQ ID NO: 830; V_{L}—SEQ ID NO: 1081), solitomab, sonepcizumab, sontuzumab, spartalizumab, stamulumab, sulesomab, suptavumab, sutimlimab, suvizumab (V_{H}—SEQ ID NO: 831; V_{L}—SEQ ID NO: 1082), suvratoxumab, tabalumab (V_{H}—SEQ ID NO: 832; V_{L}—SEQ ID NO: 1083), tacatuzumab, tadocizumab, talacotuzumab, talizumab, tamtuvetmab, tanezumab (V_{H}—SEQ ID NO: 833; V_{L}—SEQ ID NO: 1084), taplitumomab, tarextumab (V_{H}—SEQ ID NO: 834; V_{L}—SEQ ID NO: 1085), tavolimab, tefibazumab, telimomab, telisotuzumab, tenatumomab (V_{H}—SEQ ID NO: 835; V_{L}—SEQ ID NO: 1086), teneliximab, teplizumab (V_{H}—SEQ ID NO: 836; V_{L}—SEQ ID NO: 1087), tepoditamab, teprotumumab (V_{H}—SEQ ID NO: 837; V_{L}—SEQ ID NO: 1088), tesidolumab (V_{H}—SEQ ID NO: 838; V_{L}—SEQ ID NO: 1089), tetulomab, tezepelumab (V_{H}—SEQ ID NO: 839; V_{L}—SEQ ID NO: 1090), tibulizumab, tildrakizumab (V_{H}—SEQ ID NO: 364; V_{L}—SEQ ID NO: 429), tigatuzumab (V_{H}—SEQ ID NO: 840; V_{L}—SEQ ID NO: 1091), timigutuzumab, timolumab (V_{H}—SEQ ID NO: 841; V_{L}—SEQ ID NO: 1092), tiragotumab, tislelizumab, tisotumab (V_{H}—SEQ ID NO: 842; V_{L}—SEQ ID NO: 1093), tocilizumab (V_{H}—SEQ ID NO: 367; V_{L}—SEQ ID NO: 432), tomuzotuximab, toralizumab, tosatoxumab (V_{H}—SEQ ID NO: 843; V_{L}—SEQ ID NO: 1094), tositumomab, tovetumab (V_{H}—SEQ ID NO: 844; V_{L}—SEQ ID NO: 1095), tralokinumab (V_{H}—SEQ ID NO: 845; V_{L}—SEQ ID NO: 1096), trastuzumab (V_{H}—SEQ ID NO: 846; V_{L}—SEQ ID NO: 1097), tregalizumab (V_{H}—SEQ ID NO: 847; V_{L}—SEQ ID NO: 1098), tremelimumab (V_{H}—SEQ ID NO: 848; V_{L}—SEQ ID NO: 1099), trevogrumab (V_{H}—SEQ ID NO: 849; V_{L}—SEQ ID NO: 1100), tucotuzumab, tuvirumab, ublituximab (V_{H}—SEQ ID NO: 381; V_{L}—SEQ ID NO: 446), ulocuplumab (V_{H}—SEQ ID NO: 850; V_{L}—SEQ ID NO: 1101), urelumab (V_{H}—SEQ ID NO: 851; V_{L}—SEQ ID NO: 1102), urtoxazumab, ustekinumab (V_{H}—SEQ ID NO: 358; V_{L}—SEQ ID NO: 423), utomilumab, vadastuximab (V_{H}—SEQ ID NO: 852; V_{L}—SEQ ID NO: 1103), vanalimab, vandortuzumab (V_{H}—SEQ ID NO: 853; V_{L}—SEQ ID NO: 1104), vantictumab (V_{H}—SEQ ID NO: 854; V_{L}—SEQ ID NO: 1105), vanucizumab (bispecific: V_{H1}-SEQ ID NO: 855; VLi-SEQ ID NO: 1106; V_{H2}—SEQ ID NO: 357; V_{L2}—SEQ ID NO: 422), vapaliximab, varisacumab, varlilumab (V_{H}—SEQ ID NO: 856; V_{L}—SEQ ID NO: 1107), vatelizumab (V_{H}—SEQ ID NO: 857; V_{L}—SEQ ID NO: 1108), vedolizumab (V_{H}—SEQ ID NO: 858; V_{L}—SEQ ID NO: 1109), veltuzumab (V_{H}—SEQ ID NO: 859; V_{L}—SEQ ID NO: 1110), vepalimomab, vesencumab (V_{H}—SEQ ID NO: 860; V_{L}—SEQ ID NO: 1111), visilizumab (V_{H}—SEQ ID NO: 861; V_{L}—SEQ ID NO: 1112), vobarilizumab, volociximab, vonlerolizumab, vopratelimab, vorsetuzumab (V_{H}—SEQ ID NO: 862; V_{L}—SEQ ID NO: 1113), votumumab, vunakizumab, xentuzumab (V_{H}—SEQ ID NO: 863; V_{L}—SEQ ID NO: 1114), zalutumumab (V_{H}—SEQ ID NO: 864; V_{L}—SEQ ID NO: 1115), zanolimumab (V_{H}—SEQ ID NO: 865; V_{L}—SEQ ID NO: 1116), zatuximab, zenocutuzumab, ziralimumab, zolbetuximab, and zolimomab. In some embodiments, the antibody is not any one or more of the antibodies described above (e.g., does not comprise a heavy chain variable region and/or a light chain variable region of any one or more of the antibodies described above). In some embodiments, the antibody is not an anti-CTLA4 antibody and/or an anti-PD-L1 antibody.

In some embodiments, an antibody of the present disclosure comprises a light chain variable region having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of the light chain variable region of any of the antibodies described herein or known in the art (e.g., an antibody of the present disclosure comprises a light chain variable region having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the light chain variable regions disclosed in the previous paragraph). In some embodiments, an antibody of the present disclosure comprises a heavy chain variable region having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of the heavy chain variable region of any of the antibodies described herein or known in the art (e.g., an antibody of the present disclosure comprises a heavy chain variable region having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the heavy chain variable regions disclosed in the previous paragraph). In some embodiments, an antibody of the present disclosure comprises a light chain variable region and a heavy chain variable region having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of the light and heavy chain variable regions of any of the antibodies described herein or known in the art (e.g., an antibody of the present disclosure comprises a light and heavy chain variable region having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity, respectively, to the sequence of any of the light and heavy chain variable regions disclosed in the previous paragraph).

Exemplary Antibody Sequences

In some embodiments, one or more polynucleotides of the present disclosure encode an antibody (e.g., a full-length antibody, an antibody fragment, etc.) comprising a heavy chain variable region comprising an HVR-H1, HVR-H2, and/or an HVR-H3 of any of the antibodies described herein or known in the art. Methods of identifying the HVR-H1, HVR-H2, and/or HVR-H3 in a given heavy chain variable region are generally known to one of ordinary skill in the art (see e.g., the methods employed at abysis.org; Al-Lazikani et al., (1997) JMB 273, 927-948; Martin, A. C. R. (1996) Proteins 25(1):130-3; etc.). In some embodiments, one or more polynucleotides of the present disclosure encode an antibody comprising a heavy chain variable region comprising one, two, or three HVRs selected from: an HVR-H1 comprising a sequence selected from SEQ ID NOS: 1-59; an HVR-H2 comprising a sequence selected from SEQ ID NOS: 60-122; and/or an HVR-H3 comprising a sequence selected from SEQ ID NOS: 123-185. In some embodiments, one or more polynucleotides of the present disclosure encode an antibody comprising a heavy chain variable region comprising the HVR-H1, HVR-H2, and HVR-H3 of any of the antibodies depicted in Table 1. In some embodiments, one or more polynucleotides of the present disclosure encode an antibody comprising a heavy chain variable region comprising the HVR-H1, HVR-H2, and HVR-H3 of any of the heavy chain variable regions depicted in Table 2 or described in SEQ ID NOS: 355-419 or 614-865.

In some embodiments, one or more polynucleotides of the present disclosure encode an antibody comprising a light chain variable region comprising an HVR-L1, HVR-L2, and/or an HVR-L3 of any of the antibodies described herein or known in the art. Methods of identifying the HVR-L1, HVR-L2, and/or HVR-L3 in a given light chain variable region are generally known to one of ordinary skill in the art (see e.g., the methods employed at abysis.org; Al-Lazikani et al., (1997) JMB 273, 927-948; Martin, A. C. R. (1996) Proteins 25(1):130-3; etc.). In some embodiments, one or more polynucleotides of the present disclosure encode an antibody comprising a light chain variable region comprising one, two, or three HVRs selected from: an HVR-L1 comprising a sequence selected from SEQ ID NOS: 186-242; an HVR-L2 comprising a sequence selected from SEQ ID NOS: 243-294; and/or an HVR-L3 comprising a sequence selected from SEQ ID NOS: 295-354. In some embodiments, one or more polynucleotides of the present disclosure encode an antibody comprising a light chain variable region comprising the HVR-L1, HVR-L2, and HVR-L3 of any of the antibodies depicted in Table 1. In some embodiments, one or more polynucleotides of the present disclosure encode an antibody comprising a light chain variable region comprising the HVR-L1, HVR-L2, and HVR-L3 of any of the light chain variable regions depicted in Table 2 or disclosed in SEQ ID NOS: 420-482 or 866-1116.

In some embodiments, one or more polynucleotides of the present disclosure encode an antibody comprising a heavy chain variable region comprising an HVR-H1, HVR-H2, and/or an HVR-H3 of any of the antibodies described herein or known in the art, and a light chain variable region comprising an HVR-L1, HVR-L2, and/or and HVR-L3 of any of the antibodies described herein or known in the art. In some embodiments, one or more polynucleotides of the present disclosure encode an antibody comprising: a heavy chain variable region comprising one, two, or three HVRs selected from: an HVR-H1 comprising a sequence selected from SEQ ID NOS: 1-59; an HVR-H2 comprising a sequence selected from SEQ ID NOS: 60-122; and/or an HVR-H3 comprising a sequence selected from SEQ ID NOS: 123-185; and a light chain variable region comprising one, two, or three HVRs selected from: an HVR-L1 comprising a sequence selected from SEQ ID NOS: 186-242; an HVR-L2 comprising a sequence selected from SEQ ID NOS: 243-294; and/or an HVR-L3 comprising a sequence selected from SEQ ID NOS: 295-354. In some embodiments, one or more polynucleotides of the present disclosure encode an antibody comprising the HVR-H1, HVR-H2, HVR-H3, HVR-L1, HVR-L2, and HVR-L3 of any of the antibodies depicted in Table 1. In some embodiments, one or more polynucleotides of the present disclosure encode an antibody comprising the HVR-H1, HVR-H2, HVR-H3, HVR-L1, HVR-L2, and HVR-L3 of any of the heavy chain variable regions and light chain variable regions depicted in Table 2 or disclosed in SEQ ID NOS: 355-419, 420-482, 614-865, or 86-1116.

TABLE 1
HVRs of exemplary antibodies

| Ab | | | | | | |
|---|---|---|---|---|---|---|
| Name: | HVR-H1: | HVR-H2: | HVR-H3: | HVR-L1: | HVR-L2: | HVR-L3: |
| HSV- | DYAMH | AITWNSGHIDY | VSYLSTASSLD | RASQGIRNYLA | AASTLQS | QRYNRAPYT |
| AB1 | (SEQ ID NO: 1) | ADSVEG | Y | (SEQ ID NO: 186) | (SEQ ID NO: 243) | (SEQ ID NO: 295) |
| | | (SEQ ID NO: 60) | (SEQ ID NO: 123) | | | |
| HSV- | SYNMH | AIYPGNGDTSY | STYYGGDWYF | RASSSVSYIH | ATSNLAS | QQWTSNPPT |
| AB2 | (SEQ ID NO: 2) | NQKFKG | NV | (SEQ ID NO: 187) | (SEQ ID NO: 244) | (SEQ ID NO: 296) |
| | | (SEQ ID NO: 61) | (SEQ ID NO: 124) | | | |
| HSV- | NYGMN | WINTYTGEPTY | YPHYYGSSHW | SASQDISNYLN | FTSSLHS | QQYSTVPWT |
| AB3 | (SEQ ID NO: 3) | AADFKR | YFDV | (SEQ ID NO: 188) | (SEQ ID NO: 245) | (SEQ ID NO: 297) |
| | | (SEQ ID NO: 62) | (SEQ ID NO: 125) | | | |
| HSV- | TYWLG | IMSPVDSDIRYS | RRPGQGYFDF | RASQGISSWLA | AASSLQS | QQYNIYPYT |
| AB4 | (SEQ ID NO: 4) | PSFQG | (SEQ ID NO: 126) | (SEQ ID NO: 189) | (SEQ ID NO: 246) | (SEQ ID NO: 298) |
| | | (SEQ ID NO: 63) | | | | |
| HSV- | NYWMN | AINQDGSEKYY | DYYDILTDYYI | RASQSVSSSYL | GASSRAT | QQYGSSPCT |
| ABS | (SEQ ID NO: 5) | VGSVKG | HYWYFDL | A | (SEQ ID NO: 247) | (SEQ ID NO: 299) |
| | | (SEQ ID NO: 64) | (SEQ ID NO: 127) | (SEQ ID NO: 190) | | |
| HSV- | DYHIH | VINPMYGTTDY | YDYFTGTGVY | RSSRSLVHSRG | KVSNRFI | SQSTHLPFT |
| AB6 | (SEQ ID NO: 6) | NQRFKG | (SEQ ID NO: 128) | NTYLH | (SEQ ID NO: 248) | (SEQ ID NO: 300) |
| | | (SEQ ID NO: 65) | | (SEQ ID NO: 191) | | |
| HSV- | RYGIS | WISTYSGNTNY | RQLYFDY | RASQSVSSNLA | DASTRAT | QQYDNWPLT |
| AB7 | (SEQ ID NO: 7) | AQKLQG | (SEQ ID NO: 129) | (SEQ ID NO: 192) | (SEQ ID NO: 249) | (SEQ ID NO: 301) |
| | | (SEQ ID NO: 66) | | | | |
| HSV- | NYWIG | IIDPSNSYTRYSP | WYYKPFDV | TGSSNIGSGYD | GNSKRPS | ASWTDGLSLVV |
| AB8 | (SEQ ID NO: 8) | SFQG | (SEQ ID NO: 130) | VH | (SEQ ID NO: 250) | (SEQ ID NO: 302) |
| | | (SEQ ID NO: 67) | | (SEQ ID NO: 193) | | |
| HSV- | DQTIH | YIYPRDDSPKY | PDRSGYAWFIY | KASRDVAIAVA | WASTRHT | HQYSSYPFT |
| AB9 | (SEQ ID NO: 9) | NENFKG | (SEQ ID NO: 131) | (SEQ ID NO: 194) | (SEQ ID NO: 251) | (SEQ ID NO: 303) |
| | | (SEQ ID NO: 68) | | | | |
| HSV- | TYWMT | QIFPASGSADYN | GGGGFAY | RTSENIYSYLA | NAKTLAE | QHHYGIPFT |
| AB10 | (SEQ ID NO: 10) | EKFEG | (SEQ ID NO: 132) | (SEQ ID NO: 195) | (SEQ ID NO: 252) | (SEQ ID NO: 304) |
| | | (SEQ ID NO: 69) | | | | |
| HSV- | NHWMN | EIRSKSINSATH | NYYGSTYDY | RASQFVGSSIH | YASESMS | QQSHSWPFT |
| AB11 | (SEQ ID NO: 11) | YAESV | (SEQ ID NO: 133) | (SEQ ID NO: 196) | (SEQ ID NO: 253) | (SEQ ID NO: 305) |
| | | (SEQ ID NO: 70) | | | | |
| HSV- | NHWMN | EIRSKSINSATH | NYYGSTYDY | RASQFVGSSIH | YASESMS | QQSHSWPFT |
| AB11.1 | (SEQ ID NO: 12) | YAESV | (SEQ ID NO: 133) | (SEQ ID NO: 196) | (SEQ ID NO: 253) | (SEQ ID NO: 305) |
| | | (SEQ ID NO: 70) | | | | |
| HSV- | NHWMN | EIRSKSINSATH | NYYGSTYDY | RASQFVGSSIH | YASESMS | QQSHSWPFT |
| AB11.2 | (SEQ ID NO: 13) | YAESV | (SEQ ID NO: 133) | (SEQ ID NO: 196) | (SEQ ID NO: 253) | (SEQ ID NO: 305) |
| | | (SEQ ID NO: 70) | | | | |
| HSV- | ELSIH | GFDPEENEIVYA | VGSFSPLTLGL | TGSGSNIGAPY | HNNKRPS | ATVEAGLSGSV |
| AB12 | (SEQ ID NO: 14) | QRFQG | (SEQ ID NO: 134) | DVS | (SEQ ID NO: 254) | (SEQ ID NO: 306) |
| | | (SEQ ID NO: 71) | | (SEQ ID NO: 197) | | |
| HSV- | SDHAWS | YISYSGITTYNP | SLARTTAMDY | RASQDISSYLN | YTSRLHS | QQGNTLPYT |
| AB13 | (SEQ ID NO: 15) | SLKS | (SEQ ID NO: 135) | (SEQ ID NO: 198) | (SEQ ID NO: 255) | (SEQ ID NO: 307) |
| | | (SEQ ID NO: 72) | | | | |
| HSV- | DYAMH | GISWNSGRIGY | GRDSFDI | RASQGISSWLA | GASSLES | QQANSFPYT |
| AB14 | (SEQ ID NO: 1) | ADSVKG | (SEQ ID NO: 136) | (SEQ ID NO: 189) | (SEQ ID NO: 256) | (SEQ ID NO: 308) |
| | | (SEQ ID NO: 73) | | | | |
| HSV- | SYAMH | FMSYDGSNKKY | DRGIAAGGNYY | RASQSVYSYLA | DASNRAT | QQRSNWPPFT |
| AB15 | (SEQ ID NO: 16) | ADSVKG | YYGMDV | (SEQ ID NO: 199) | (SEQ ID NO: 257) | (SEQ ID NO: 309) |
| | | (SEQ ID NO: 74) | (SEQ ID NO: 137) | | | |
| HSV- | DYGMN | WINTYIGEPIYA | GYRSYAMDY | KASQNVGTNV | SASFLYS | QQYNIYPLT |
| AB16 | (SEQ ID NO: 17) | DSVKG | (SEQ ID NO: 138) | A | (SEQ ID NO: 258) | (SEQ ID NO: 310) |
| | | (SEQ ID NO: 75) | | (SEQ ID NO: 200) | | |
| HSV- | SFGMH | YISSDSSAIYYA | GRENIYYGSRL | KASQNVDTNV | SASYRYS | QQYNNYPFT |
| AB17 | (SEQ ID NO: 18) | DTVKG (SEQ ID | DY | A | (SEQ ID NO: 259) | (SEQ ID NO: 311) |
| | | NO: 76) | (SEQ ID NO: 139) | (SEQ ID NO: 201) | | |
| HSV- | SYTMH | FISYDGNNKYY | TGWLGPFDY | RASQSVGSSYL | GAFSRAT | QQYGSSPWT |
| AB18 | (SEQ ID NO: 19) | ADSVKG | (SEQ ID NO: 140) | A | (SEQ ID NO: 260) | (SEQ ID NO: 312) |
| | | (SEQ ID NO: 77) | | (SEQ ID NO: 202) | | |
| HSV- | DYVIH | YINPYDDDTTY | RGNSYDGYFDY | RASQNVGTAVA | SASNRYT | QQYTNYPMYT |
| AB19 | (SEQ ID NO: 20) | NQKFKG | SMDY | (SEQ ID NO: 203) | (SEQ ID NO: 261) | (SEQ ID NO: 313) |
| | | (SEQ ID NO: 78) | (SEQ ID NO: 141) | | | |
| HSV- | DSWIH | WISPYGGSTYY | RHWPGGFDY | RASQDVSTAVA | SASFLYS | QQYLYHPAT |
| AB20 | (SEQ ID NO: 21) | ADSVKG | (SEQ ID NO: 142) | (SEQ ID NO: 204) | (SEQ ID NO: 258) | (SEQ ID NO: 314) |
| | | (SEQ ID NO: 79) | | | | |
| HSV- | RYWMS | NIKQDGSEKYY | EGGWFGELAFD | RASQRVSSSYL | DASSRAT | QQYGSLPWT |
| AB21 | (SEQ ID NO: 22) | VDSVKG | Y | A | (SEQ ID NO: 262) | (SEQ ID NO: 315) |
| | | (SEQ ID NO: 80) | (SEQ ID NO: 143) | (SEQ ID NO: 205) | | |
| HSV- | NSGMH | VIWYDGSKRYY | NDDY | RASQSVSSYLA | DASNRAT | QQSSNWPRT |
| AB22 | (SEQ ID NO: 23) | ADSVKG | (SEQ ID NO: 144) | (SEQ ID NO: 206) | (SEQ ID NO: 257) | (SEQ ID NO: 316) |
| | | (SEQ ID NO: 81) | | | | |
| HSV- | NYYMY | GINPSNGGTISF | RDYRFDMGFD | RASKGVSTSGY | LASYLES | QHSRDLPLT |
| AB23 | (SEQ ID NO: 24) | NEKFKN | Y | SYLH | (SEQ ID NO: 263) | (SEQ ID NO: 317) |
| | | (SEQ ID NO: 82) | (SEQ ID NO: 145) | (SEQ ID NO: 207) | | |
| HSV- | VYGMN | IIWYDGDNQYY | DLRTGPFDY | RASQSIGSSLH | YASQSFS | HQSSSLPFT |
| AB24 | (SEQ ID NO: 25) | ADSVKG | (SEQ ID NO: 146) | (SEQ ID NO: 208) | (SEQ ID NO: 264) | (SEQ ID NO: 318) |
| | | (SEQ ID NO: 83) | | | | |
| HSV- | SYNMH | AIYPGNGDTSY | VVYYSNSYWY | RASSSVSYMH | APSNLAS | QQWSFNPPT |
| AB25 | (SEQ ID NO: 2) | NQKFKG | FDV | (SEQ ID NO: 209) | (SEQ ID NO: 265) | (SEQ ID NO: 319) |
| | | (SEQ ID NO: 61) | (SEQ ID NO: 147) | | | |
| HSV- | DYAMH | TISWNSGSIGYA | DIQYGNYYYG | RASQSVSSYLA | DASNRAT | QQRSNWPIT |
| AB26 | (SEQ ID NO: 1) | DSVKG | MDV | (SEQ ID NO: 206) | (SEQ ID NO: 257) | (SEQ ID NO: 320) |
| | | (SEQ ID NO: 84) | (SEQ ID NO: 148) | | | |
| HSV- | SYNMH | GIYPGNGDTSY | YDYNYAMDY | RASSSVSYMH | ATSNLAS | QQWTFNPPT |
| AB27 | (SEQ ID NO: 2) | NQKFKG | (SEQ ID NO: 149) | (SEQ ID NO: 209) | (SEQ ID NO: 244) | (SEQ ID NO: 321) |
| | | (SEQ ID NO: 85) | | | | |
| HSV- | DFYMN | FIRDKAKGYTT | EGHTAAPFDY | KASQNIDKYLN | NTNNLQT | LQHISRPRT |
| AB28 | (SEQ ID NO: 26) | EYNPSVKG | (SEQ ID NO: 150) | (SEQ ID NO: 210) | (SEQ ID NO: 266) | (SEQ ID NO: 322) |
| | | (SEQ ID NO: 86) | | | | |
| HSV- | AYEMK | VIGPSGGFTFYA | EGDNDAFDI | RASQSVSSYLA | DASNRAT | QQRSNWPMYT |
| AB29 | (SEQ ID NO: 27) | DSVKG | (SEQ ID NO: 151) | (SEQ ID NO: 206) | (SEQ ID NO: 257) | (SEQ ID NO: 323) |
| | | (SEQ ID NO: 87) | | | | |
| HSV- | DTYIH | RIDPANGYTKY | EGYYGNYGVY | KTSQDINKYMA | YTSALQP | LQYDNLWT |
| AB30 | (SEQ ID NO: 28) | DPKFQG | AMDY | (SEQ ID NO: 211) | (SEQ ID NO: 267) | (SEQ ID NO: 324) |
| | | (SEQ ID NO: 88) | (SEQ ID NO: 152) | | | |
| HSV- | NYWIQ | EILPGSGSTEYT | YFFGSSPNWYF | GASENIYGALN | GATNLAD | QNVLNTPLT |
| AB31 | (SEQ ID NO: 29) | ENFKD | DV | (SEQ ID NO: 212) | (SEQ ID NO: 268) | (SEQ ID NO: 325) |
| | | (SEQ ID NO: 89) | (SEQ ID NO: 153) | | | |
| HSV- | HDHAWS | FISYSGITNYNP | SLARTTAMDY | QASTDISSHLN | YGSHLLS | GQGNRLPYT |
| AB32 | (SEQ ID NO: 30) | SLQG | (SEQ ID NO: 135) | (SEQ ID NO: 213) | (SEQ ID NO: 269) | (SEQ ID NO: 326) |
| | | (SEQ ID NO: 90) | | | | |
| HSV- | NYWIA | IIYPGDSDIRYSP | HDIEGFDY | RASQSVSSSFFA | GASSRAT | QQYDSSAIT |
| AB33 | (SEQ ID NO: 31) | SFQG | (SEQ ID NO: 154) | (SEQ ID NO: 214) | (SEQ ID NO: 247) | (SEQ ID NO: 327) |
| | | (SEQ ID NO: 91) | | | | |
| HSV- | NNAIN | GIIPMFGTAKYS | SRDLLLFPHHA | QGDSLRSYYAS | GKNNRPS | SSRDSSGNHWV |
| AB34 | (SEQ ID NO: 32) | QNFQG | LSP | (SEQ ID NO: 215) | (SEQ ID NO: 270) | (SEQ ID NO: 328) |
| | | (SEQ ID NO: 92) | (SEQ ID NO: 155) | | | |
| HSV- | RYWMH | AIYPGNSDTSY | DYGYYFDF | SASSSRSYMQ | DTSKLAS | HQRSSYT |
| AB35 | (SEQ ID NO: 33) | NQKFEG | (SEQ ID NO: 156) | (SEQ ID NO: 216) | (SEQ ID NO: 271) | (SEQ ID NO: 329) |
| | | (SEQ ID NO: 93) | | | | |
| HSV- | SYRMH | YINPSTGYTEYN | GGGVFDY | SASSSISYMH | TTSNLAS | HQRSTYPLT |
| AB36 | (SEQ ID NO: 34) | QKFKD | (SEQ ID NO: 157) | (SEQ ID NO: 217) | (SEQ ID NO: 272) | (SEQ ID NO: 330) |
| | | (SEQ ID NO: 94) | | | | |
| HSV- | DYAMT | SISGSGGNTYY | DRLSITIRPRYY | RSSQSLLYSIGY | LGSNRAS | MQALQTPYT |
| AB37 | (SEQ ID NO: 35) | ADSVKG | GLDV | NYLD | (SEQ ID NO: 273) | (SEQ ID NO: 331) |
| | | (SEQ ID NO: 95) | (SEQ ID NO: 158) | (SEQ ID NO: 218) | | |
| HSV- | NYAMN | TISGSGGTTNYA | DSNWGNFDL | KSSQSVLYRSN | WASTRES | QQYYTTPYT |
| AB38 | (SEQ ID NO: 36) | DSVKG | (SEQ ID NO: 159) | NRNFLG | (SEQ ID NO: 274) | (SEQ ID NO: 332) |
| | | (SEQ ID NO: 96) | | (SEQ ID NO: 219) | | |
| HSV- | DYWMQ | TIYPGDGDTGY | GDYYGSNSLDY | KASQDVSTVVA | SASYRYI | QQHYSPPYT |
| AB39 | (SEQ ID NO: 37) | AQKFQG | (SEQ ID NO: 160) | (SEQ ID NO: 220) | (SEQ ID NO: 275) | (SEQ ID NO: 333) |
| | | (SEQ ID NO: 97) | | | | |
| HSV- | NFGMT | GISGGGRDTYF | WGNIYFDY | RASLSINTFLN | AASSLHG | QQSSNTPFT |
| AB40 | (SEQ ID NO: 38) | ADSVKG | (SEQ ID NO: 161) | (SEQ ID NO: 221) | (SEQ ID NO: 276) | (SEQ ID NO: 334) |
| | | (SEQ ID NO: 98) | | | | |
| HSV- | SYDMS | YISSGGGITYFP | HYFGSSGPFAY | RASENIFSYLA | NTKTLAE | QHHYGTPFT |
| AB41 | (SEQ ID NO: 39) | DTVQG | (SEQ ID NO: 162) | (SEQ ID NO: 222) | (SEQ ID NO: 277) | (SEQ ID NO: 335) |
| | | (SEQ ID NO: 99) | | | | |
| HSV- | SNVIS | GVIPIVDIANYA | TLGLVLDAMD | RASQSLGSSYL | GASSRAP | QQYADSPIT |
| AB42 | (SEQ D NO: 40) | QRFKG | Y | A | (SEQ ID NO: 278) | (SEQ ID NO: 336) |
| | | (SEQ ID NO: 100) | (SEQ ID NO: 163) | (SEQ ID NO: 223) | | |
| HSV- | DSSIN | MIWGDGRIDYA | DGYFPYAMDF | RASESVDSYGQ | LASNLES | QQNAEDSRT |
| AB43 | (SEQ ID NO: 41) | DALKS | (SEQ ID NO: 164) | SYMH | (SEQ ID NO: 279) | (SEQ ID NO: 337) |
| | | (SEQ ID NO: 101) | | (SEQ ID NO: 224) | | |
| HSV- | SYWIH | MIDPSDGETRL | LKEYGNYDSFY | HASQNIDVWLS | KASNLHT | QQAHSYPFT |
| AB44 | (SEQ ID NO: 42) | NQRFQG | FDV | (SEQ ID NO: 225) | (SEQ ID NO: 280) | (SEQ ID NO: 338) |
| | | (SEQ ID NO: 102) | (SEQ ID NO: 165) | | | |
| HSV- | SYNMH | YIYPGNGATNY | GDSVPFAY | SAHSSVSFMH | STSSLAS | QQRSSFPLT |
| AB45 | (SEQ ID NO: 2) | NQKFQG | (SEQ ID NO: 166) | (SEQ ID NO: 226) | (SEQ ID NO: 281) | (SEQ ID NO: 339) |
| | | (SEQ ID NO: 103) | | | | |
| HSV- | SYNMH | YIYPGNGATNY | GDSVPFAY | SAHSSVSFMH | STSSLAS | QQRSSFPLT |
| AB46 | (SEQ ID NO: 2) | NQKFQG | (SEQ ID NO: 166) | (SEQ ID NO: 226) | (SEQ ID NO: 281) | (SEQ ID NO: 339) |
| | | (SEQ ID NO: 103) | | | | |
| HSV- | SYGMH | IIWYDGSNKYY | VATSGDFDYYG | RASQRISTYLA | DASKRAT | QQRSNWPLT |
| AB47 | (SEQ ID NO: 43) | ADSVKG | MDV | (SEQ ID NO: 227) | (SEQ ID NO: 282) | (SEQ ID NO: 340) |
| | | (SEQ ID NO: 104) | (SEQ ID NO: 167) | | | |
| HSV- | DYGMT | GIHWHGKRTGY | GGMSTGDWFD | RASQSINSYLN | VASSLQS | QQSYSTPPIT |
| AB48 | (SEQ ID NO: 44) | ADSVKG | P | (SEQ ID NO: 228) | (SEQ ID NO: 283) | (SEQ ID NO: 341) |
| | | (SEQ ID NO: 105) | (SEQ ID NO: 168) | | | |
| HSV- | NYAMS | TISSGGSHTYYL | LFTGYAMDY | TASSSVSSSYLH | STSNLAS | HQYYRLPPIT |
| AB49 | (SEQ ID NO: 45) | DSVKG | (SEQ ID NO: 169) | (SEQ ID NO: 229) | (SEQ ID NO: 284) | (SEQ ID NO: 342) |
| | | (SEQ ID NO: 106) | | | | |
| HSV- | RSAMN | GISGSGGRTYY | DSYTTSWYGG | RASQGIFSWLA | AASSLQS | QQANSVPIT |
| AB50 | (SEQ ID NO: 46) | ADSVKG | MDV | (SEQ ID NO: 230) | (SEQ ID NO: 246) | (SEQ ID NO: 343) |
| | | (SEQ ID NO: 107) | (SEQ ID NO: 170) | | | |
| HSV- | NYNIH | AIYPGNGDAPY | ANWDVAFAY | KASQDIDRYMA | DTSTLQS | LQYDNLWT |
| AB51 | (SEQ ID NO: 47) | SQKFQG | (SEQ ID NO: 171) | (SEQ ID NO: 231) | (SEQ ID NO: 285) | (SEQ ID NO: 324) |
| | | (SEQ ID NO: 108) | | | | |
| HSV- | DYAMN | AISGDGGSTYY | DLRNTIFGVVIP | RASQSIRSWLA | KASSLES | QQYNSYSYT |
| AB52 | (SEQ ID NO: 48) | ADSVKG | DAFDI | (SEQ ID NO: 232) | (SEQ ID NO: 286) | (SEQ ID NO: 344) |
| | | (SEQ ID NO: 109) | (SEQ ID NO: 172) | | | |
| HSV- | ELSIH | GFDPEDGETIYA | IFGVVTNFDN | RASQAIRNDLG | AAFNLQS | QQYNRYPWT |
| AB53 | (SEQ ID NO: 14) | QKFQG | (SEQ ID NO: 173) | (SEQ ID NO: 233) | (SEQ ID NO: 287) | (SEQ ID NO: 345) |
| | | (SEQ ID NO: 110) | | | | |
| HSV- | SHFWS | YILYTGGTSFNP | ARSGITFTGIIVP | RASQSVSSSYL | GASSRAT | QQYGSSPWT |
| AB54 | (SEQ ID NO: 49) | SLKS | GSFDI | A | (SEQ ID NO: 247) | (SEQ ID NO: 312) |
| | | (SEQ ID NO: 111) | (SEQ ID NO: 174) | (SEQ ID NO: 190) | | |
| HSV- | SDAMY | VIYYDGNYQYY | LNWDYWYLDL | RASQSISSYLN | AASSLQS | QQSYSTPPIT |
| AB55 | (SEQ ID NO: 50) | EDSVKG | (SEQ ID NO: 175) | (SEQ ID NO: 234) | (SEQ ID NO: 246) | (SEQ ID NO: 341) |
| | | (SEQ ID NO: 112) | | | | |
| HSV- | DYTMH | GISWNSGSIGYA | DNSGYGHYYY | RASQSVSSNLA | GASTRAT | QHYINWPLT |
| AB56 | (SEQ ID NO: 51) | DSVKG | GMDV | (SEQ ID NO: 192) | (SEQ ID NO: 288) | (SEQ ID NO: 346) |
| | | (SEQ ID NO: 113) | (SEQ ID NO: 176) | | | |
| HSV- | DYAMH | VISWNSDSIGYA | DNHYGSGSYYY | RASQSVSSNLA | GASTRAT | QHYINWPLT |
| AB57 | (SEQ ID NO: 1) | DSVKG | YQYGMDV | (SEQ ID NO: 192) | (SEQ ID NO: 288) | (SEQ ID NO: 346) |
| | | (SEQ ID NO: 114) | (SEQ ID NO: 177) | | | |
| HSV- | NYNIN | LISGSSSYIYYA | RTLSYYVMDV | RASQGISNYLA | AASTLQS | QKYNSAPYT |
| AB58 | (SEQ ID NO: 52) | DSVKG | (SEQ ID NO: 178) | (SEQ ID NO: 235) | (SEQ ID NO: 243) | (SEQ ID NO: 347) |
| | | (SEQ ID NO: 115) | | | | |
| HSV- | SSYWT | YIYYSGSSNYNP | EGNVDTTMIFD | RASQGIRNDLG | AASSLQS | LQDFNYPWT |
| AB59 | (SEQ ID NO: 53) | SLKS | Y | (SEQ ID NO: 236) | (SEQ ID NO: 246) | (SEQ ID NO: 348) |
| | | (SEQ ID NO: 116) | (SEQ ID NO: 179) | | | |
| HSV- | SFGMH | VISFDGSIKYSV | DRLNYYDSSGY | SGSSSNIGNNYV | DNNKRPS | GTWDSRLSAVV |
| AB60 | (SEQ ID NO: 18) | DSVKG | YHYKYYGMAV | S | (SEQ ID NO: 289) | (SEQ ID NO: 349) |
| | | (SEQ ID NO: 117) | (SEQ ID NO: 180) | (SEQ ID NO: 237) | | |
| HSV- | SSYIN | TINPVSGSTSYA | TGTSSDVGSYN | YVN | GVSKRPS | GTFAGGSYYGV |
| AB61 | (SEQ ID NO: 55) | QKFQG | GGWFDY | (SEQ ID NO: 238) | (SEQ ID NO: 290) | (SEQ ID NO: 350) |
| | | (SEQ ID NO: 118) | (SEQ ID NO: 181) | | | |
| HSV- | HYGMN | WINTYTGEPTY | YPYYYGTSHW | SASQDISNYLN | FTSSLHS | QQYSTVPWT |
| AB62 | (SEQ ID NO: 56) | AADFKR | YFDV | (SEQ ID NO: 188) | (SEQ ID NO: 245) | (SEQ ID NO: 297) |
| | | (SEQ ID NO: 62) | (SEQ ID NO: 182) | | | |
| HSV- | WYWLE | EIDPGTFITNYN | FSHFSGSNYDY | RASQSIGTNIH | YASESIS | QQSWSWPTT |
| AB63 | (SEQ ID NO: 57) | EKFKA | FDY | (SEQ ID NO: 240) | (SEQ ID NO: 292) | (SEQ ID NO: 352) |
| | | (SEQ ID NO: 120) | (SEQ ID NO: 183) | | | |
| HSV- | SGYSWN | SITYDGSTNYA | GSHYFGHWHF | RASQSVDYDGD | AASYLES | QQSHEDPYT |
| AB64 | (SEQ ID NO: 58) | DSVKG | AV | SYMN | (SEQ ID NO: 293) | (SEQ ID NO: 353) |
| | | (SEQ ID NO: 121) | (SEQ ID NO: 184) | (SEQ ID NO: 241) | | |
| HSV- | SYGMH | VISYEESNRYH | DGGIAAPGPDY | RSSQSLLYSNG | LGSNRAS | MQARQTPFT |
| AB65 | (SEQ ID NO: 43) | ADSVKG | YNYLD | (SEQ ID NO: 242) | (SEQ ID NO: 273) | (SEQ ID NO: 354) |
| | | (SEQ ID NO: 122) | (SEQ ID NO: 185) | | | |
| HSV- | DYNIH | YIYPNNGDNGY | GRLRYFDV | RASESVDNYGH | LASNLES | QQYNEDPPT |
| AB66 | (SEQ ID NO: 54) | NQKFRG | (SEQ ID NO: 607) | SFMH | (SEQ ID NO: 279) | (SEQ ID NO: 351) |
| | | (SEQ ID NO: 119) | | (SEQ ID NO: 239) | | |
| HSV- | SYWMS | NIKQDGSEKYY | DRGSLYY | RPSQGINWELA | DASSLEQ | QQFNSYPLT |
| AB67 | (SEQ ID NO: 59) | VDSVKG | (SEQ ID NO: 608) | (SEQ ID NO: 610) | (SEQ ID NO: 291) | (SEQ ID NO: 612) |
| | | (SEQ ID NO: 80) | | | | |
| HSV- | SYAMS | GIIPIFGTVNYA | RRGAKFDY | SGSTSNIGSHYV | RNHQRPS | AVWDDTLSGW |
| AB68 | (SEQ ID NO: 605) | QKFQG | (SEQ ID NO: 609) | V | (SEQ ID NO: 294) | V |
| | | (SEQ ID NO: 606) | | (SEQ ID NO: 611) | | (SEQ ID NO: 613) |

In some embodiments, one or more polynucleotides of the present disclosure encode an antibody comprising a heavy chain variable region comprising: an HVR-H1 comprising the sequence of SEQ ID NO: 1; an HVR-H2 comprising the sequence of SEQ ID NO: 60; and an HVR-H3 comprising the sequence of SEQ ID NO: 123. In some embodiments, one or more polynucleotides of the present disclosure encode an antibody comprising a heavy chain variable region comprising: an HVR-H1 comprising the sequence of SEQ ID NO: 2; an HVR-H2 comprising the sequence of SEQ ID NO: 61; and an HVR-H3 comprising the sequence of SEQ ID NO: 124. In some embodiments, one or more polynucleotides of the present disclosure encode an antibody comprising a heavy chain variable region comprising: an HVR-H1 comprising the sequence of SEQ ID NO: 3; an HVR-H2 comprising the sequence of SEQ ID NO: 62; and an HVR-H3 comprising the sequence of SEQ ID NO: 125.

In some embodiments, one or more polynucleotides of the present disclosure encode an antibody comprising a light chain variable region comprising: an HVR-L1 comprising the sequence of SEQ ID NO: 186; an HVR-L2 comprising the sequence of SEQ ID NO: 243; and an HVR-L3 comprising the sequence of SEQ ID NO: 295. In some embodiments, one or more polynucleotides of the present disclosure encode an antibody comprising a light chain variable region comprising: an HVR-L1 comprising the sequence of SEQ ID NO: 187; an HVR-L2 comprising the sequence of SEQ ID NO: 244; and an HVR-L3 comprising the sequence of SEQ ID NO: 296. In some embodiments, one or more polynucleotides of the present disclosure encode an antibody comprising a light chain variable region comprising: an HVR-L1 comprising the sequence of SEQ ID NO: 188; an HVR-L2 comprising the sequence of SEQ ID NO: 245; and an HVR-L3 comprising the sequence of SEQ ID NO: 2297.

In some embodiments, one or more polynucleotides of the present disclosure encode an antibody comprising: an HVR-H1 comprising the sequence of SEQ ID NO: 1; an HVR-H2 comprising the sequence of SEQ ID NO: 60; an HVR-H3 comprising the sequence of SEQ ID NO: 123; an HVR-L1 comprising the sequence of SEQ ID NO: 186; an HVR-L2 comprising the sequence of SEQ ID NO: 243; and an HVR-L3 comprising the sequence of SEQ ID NO: 295. In some embodiments, one or more polynucleotides of the present disclosure encode an antibody comprising: an HVR-H1 comprising the sequence of SEQ ID NO: 2; an HVR-H2 comprising the sequence of SEQ ID NO: 61; an HVR-H3 comprising the sequence of SEQ ID NO: 124; an HVR-L1 comprising the sequence of SEQ ID NO: 187; an HVR-L2 comprising the sequence of SEQ ID NO: 244; and an HVR-L3 comprising the sequence of SEQ ID NO: 296. In some embodiments, one or more polynucleotides of the present disclosure encode an antibody comprising: an HVR-H1 comprising the sequence of SEQ ID NO: 3; an HVR-H2 comprising the sequence of SEQ ID NO: 62; an HVR-H3 comprising the sequence of SEQ ID NO: 125; an HVR-L1 comprising the sequence of SEQ ID NO: 188; an HVR-L2 comprising the sequence of SEQ ID NO: 245; and an HVR-L3 comprising the sequence of SEQ ID NO: 297.

In some embodiments, one or more polynucleotides of the present disclosure encode an antibody comprising a heavy chain variable region of any of the antibodies described herein or known in the art. In some embodiments, one or more polynucleotides of the present disclosure encode an antibody comprising a heavy chain variable region comprising a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 355-419 or 614-865. In some embodiments, one or more polynucleotides of the present disclosure encode an antibody comprising a heavy chain variable region comprising a sequence selected from SEQ ID NOS: 355-419 or 614-865. In some embodiments, one or more polynucleotides of the present disclosure encode an antibody comprising the heavy chain variable region of any of the antibodies depicted in Table 2.

In some embodiments, one or more polynucleotides of the present disclosure encode an antibody comprising a light chain variable region of any of the antibodies described herein or known in the art. In some embodiments one or more polynucleotides of the present disclosure encode an antibody comprising a light chain variable region comprising a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 420-482 or 866-1116. In some embodiments, one or more polynucleotides of the present disclosure encode an antibody comprising a light chain variable region comprising a sequence selected from SEQ ID NOS: 420-482 or 866-1116. In some embodiments, one or more polynucleotides of the present disclosure encode an antibody comprising the light chain variable region of any of the antibodies depicted in Table 2.

In some embodiments, one or more polynucleotides of the present disclosure encode an antibody comprising a heavy chain variable region and a light chain variable region of any of the antibodies described herein or known in the art. In some embodiments, one or more polynucleotides of the present disclosure encode an antibody comprising: a heavy chain variable region comprising a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 355-419 or 614-865; and a light chain variable region comprising a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 420-482 or 866-1116. In some embodiments, one or more polynucleotides of the present disclosure encode an antibody comprising a heavy chain variable region comprising a sequence selected from SEQ ID NOS: 355-419 or 614-865, and a light chain variable region comprising a sequence selected from SEQ ID NOS: 420-482 or 866-1116. In some embodiments, one or more polynucleotides of the present disclosure encode an antibody comprising a heavy chain variable region and a light chain variable region each independently having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the heavy and light chain variable regions of any of the antibodies depicted in Table 2. In some embodiments, one or more polynucleotides of the present disclosure encode an antibody comprising the heavy chain variable region and the light chain variable region of any of the antibodies depicted in Table 2.

TABLE 2
Heavy and light chain variable regions of exemplary antibodies

| AB | | |
|---|---|---|
| Name: | VH: | VL: |
| HSV- | EVQLVESGGGLVQPGRSLRLSCAASGFTFDDYAMHWVRQAPG | DIQMTQSPSSLSASVGDRVTITCRASQGIRNYLA |
| AB1 | KGLEWVSAITWNSGHIDYADSVEGRFTISRDNAKNSLYLQMNS | WYQQKPGKAPKLLIYAASTLQSGVPSRFSGSGSG |
| | LRAEDTAVYYCAKVSYLSTASSLDYWGQGTLVTVSS (SEQ ID | TDFTLTISSLQPEDVATYYCQRYNRAPYTEGQGT |
| | NO: 355) | KVEIK (SEQ ID NO: 420) |
| HSV- | QVQLQQPGAELVKPGASVKMSCKASGYTFTSYNMHWVKQTPG | QIVLSQSPAILSASPGEKVTMTCRASSSVSYIHWF |
| AB2 | RGLEWIGAIYPGNGDTSYNQKFKGKATLTADKSSSTAYMQLSSL | QQKPGSSPKPWIYATSNLASGVPVRFSGSGSGTS |
| | TSEDSAVYYCARSTYYGGDWYFNVWGAGTTVTVSA (SEQ ID | YSLTISRVEAEDAATYYCQQWTSNPPTFGGGTK |
| | NO: 356) | LEIK (SEQ ID NO: 421) |
| HSV- | EVQLVESGGGLVQPGGSLRLSCAASGYTFTNYGMNWVRQAPG | DIQMTQSPSSLSASVGDRVTITCSASQDISNYLN |
| AB3 | KGLEWVGWINTYTGEPTYAADFKRRFTFSLDTSKSTAYLQMNS | WYQQKPGKAPKVLIYFTSSLHSGVPSRFSGSGSG |
| | LRAEDTAVYYCAKYPHYYGSSHWYFDVWGQGTLVTVSS (SEQ | TDFTLTISSLQPEDFATYYCQQYSTVPWTFGQGT |
| | ID NO: 357) | KVEIK (SEQ ID NO: 422) |
| HSV- | EVQLVQSGAEVKKPGESLKISCKGSGYSFITYWLGWVRQMPGK | DIQMTQSPSSLSASVGDRVTITCRASQGISSWLA |
| AB4 | GLDWIGIMSPVDSDIRYSPSFQGQVTMSVDKSITTAYLQWNSLK | WYQQKPEKAPKSLIYAASSLQSGVPSRFSGSGSG |
| | ASDTAMYYCARRRPGQGYFDFWGQGTLVTVSS (SEQ ID NO: | TDFTLTISSLQPEDFATYYCQQYNIYPYTFGQGT |
| | 358) | KLEIK (SEQ ID NO: 423) |
| HSV- | EVQLVESGGGLVQPGGSLRLSCAASGFTESNYWMNWVRQAPG | EIVLTQSPGTLSLSPGERATLSCRASQSVSSSYLA |
| ABS | KGLEWVAAINQDGSEKYYVGSVKGRFTISRDNAKNSLYLQMNS | WYQQKPGQAPRLLIYGASSRATGIPDRFSGSGSG |
| | LRVEDTAVYYCVRDYYDILTDYYIHYWYFDLWGRGTLVTVSS | TDFTLTISRLEPEDFAVYYCQQYGSSPCTFGQGT |
| | (SEQ ID NO: 359) | RLEIK (SEQ ID NO: 424) |
| HSV- | QVQLVQSGAEVKKPGSSVKVSCKASGYSFTDYHIHWVRQAPGQ | DIVMTQTPLSLSVTPGQPASISCRSSRSLVHSRGN |
| AB6 | GLEWMGVINPMYGTTDYNQRFKGRVTITADESTSTAYMELSSL | TYLHWYLQKPGQSPQLLIYKVSNRFIGVPDRFSG |
| | RSEDTAVYYCARYDYFTGTGVYWGQGTLVTVSS (SEQ ID NO: | SGSGTDFTLKISRVEAEDVGVYYCSQSTHLPFTF |
| | 360) | GQGTKLEIK (SEQ ID NO: 425) |
| HSV- | QVQLVQSGAEVKKPGASVKVSCKASGYTFTRYGISWVRQAPGQ | EIVMTQSPATLSVSPGERATLSCRASQSVSSNLA |
| AB7 | GLEWMGWISTYSGNTNYAQKLQGRVTMTTDTSTSTAYMELRS | WFQQKPGQAPRPLIYDASTRATGVPARFSGSGSG |
| | LRSDDTAVYYCARRQLYFDYWGQGTLVTVSS | TDFTLTISSLQSEDFAVYYCQQYDNWPLTEGGGT |
| | (SEQ ID NO: 361) | KVEIK (SEQ ID NO: 426) |
| HSV- | EVQLVQSGAEVKKPGESLKISCKGSGYSFSNYWIGWVRQMPGK | QSVLTQPPSVSGAPGQRVTISCTGSSSNIGSGYDV |
| AB8 | GLEWMGIIDPSNSYTRYSPSFQGQVTISADKSISTAYLQWSSLKA | HWYQQLPGTAPKLLIYGNSKRPSGVPDRFSGSKS |
| | SDTAMYYCARWYYKPFDVWGQGTLVTVSS (SEQ ID NO: 362) | GTSASLAITGLQSEDEADYYCASWTDGLSLVVF |
| | | GGGTKLTVL (SEQ ID NO: 427) |
| HSV- | QVQLVQSGAEVKKPGSSVKVSCKASGYTFTDQTIHWMRQAPG | DIQMTQSPSSLSASVGDRVTITCKASRDVAIAVA |
| AB9 | QGLEWIGYIYPRDDSPKYNENFKGKVTITADKSTSTAYMELSSL | WYQQKPGKVPKLLIYWASTRHTGVPSRFSGSGS |
| | RSEDTAVYYCAIPDRSGYAWFIYWGQGTLVTVSS (SEQ ID NO: | RTDFTLTISSLQPEDVADYFCHQYSSYPFTEGSGT |
| | 363) | KLEIK (SEQ ID NO: 428) |
| HSV- | QVQLVQSGAEVKKPGASVKVSCKASGYIFITYWMTWVRQAPG | DIQMTQSPSSLSASVGDRVTITCRTSENIYSYLAW |
| AB10 | QGLEWMGQIFPASGSADYNEKFEGRVTMTTDTSTSTAYMELRS | YQQKPGKAPKLLIYNAKTLAEGVPSRFSGSGSGT |
| | LRSDDTAVYYCARGGGGFAYWGQGTLVTVSS (SEQ ID NO: | DFTLTISSLQPEDFATYYCQHHYGIPFTEGQGTKV |
| | 364) | EIK (SEQ ID NO: 429) |
| HSV- | EVKLEESGGGLVQPGGSMKLSCVASGFIFSNHWMNWVRQSPEK | DILLTQSPAILSVSPGERVSFSCRASQFVGSSIHW |
| AB11 | GLEWVAEIRSKSINSATHYAESVKGRFTISRDDSKSAVYLQMTD | YQQRTNGSPRLLIKYASESMSGIPSRFSGSGSGTD |
| | LRTEDTGVYYCSRNYYGSTYDYWGQGTTLTVSS (SEQ ID NO: | FTLSINTVESEDIADYYCQQSHSWPFTFGSGTNLE |
| | 365) | VK (SEQ ID NO: 430) |
| HSV- | QVQLVQSGAEVKKPGASVKVSCKVSGYTLTELSIHWVRQAPGK | QSVLTQPPSVSGAPGQRVTISCTGSGSNIGAPYD |
| AB12 | GLEWMGGFDPEENEIVYAQRFQGRVTMTEDTSTDTAYMELSSL | VSWYQQLPGTAPKLLIYHNNKRPSGVPDRFSGS |
| | RSEDTAVYYCAIVGSFSPLTLGLWGQGTMVTVSS (SEQ ID NO: | KSGTSASLAITGLQAEDEADYYCATVEAGLSGS |
| | 366) | VFOGGTKLTVL (SEQ ID NO: 431) |
| HSV- | EVQLQESGPGLVRPSQTLSLTCTVSGYSITSDHAWSWVRQPPGR | DIQMTQSPSSLSASVGDRVTITCRASQDISSYLN |
| AB13 | GLEWIGYISYSGITTYNPSLKSRVTMLRDTSKNQFSLRLSSVTAA | WYQQKPGKAPKLLTYYTSRLHSGVPSRFSGSGS |
| | DTAVYYCARSLARTTAMDYWGQGSLVTVSS | GTDFTFTISSLQPEDIATYYCQQGNTLPYTEGQGT |
| | (SEQ ID NO: 367) | KVEIK (SEQ ID NO: 432) |
| HSV- | EVQLVESGGGLVQPGRSLRLSCAASRFTFDDYAMHWVRQAPG | DIQMTQSPSSVSASVGDRVTITCRASQGISSWLA |
| AB14 | KGLEWVSGISWNSGRIGYADSVKGRFTISRDNAENSLFLQMNGL | WYQQKPGKAPKWYGASSLESGVPSRFSGSGSG |
| | RAEDTALYYCAKGRDSFDIWGQGTMVTVSS (SEQ ID NO: | TDFTLTISSLQPEDFASYYCQQANSFPYTEGQGT |
| | 368) | KLEIK (SEQ ID NO: 433) |
| HSV- | QVQLVESGGGVVQPGRSLRLSCAASGFIFSSYAMHWVRQAPGN | EIVLTQSPATLSLSPGERATLSCRASQSVYSYLA |
| AB15 | GLEWVAFMSYDGSNKKYADSVKGRFTISRDNSKNTLYLQMNSL | WYQQKPGQAPRLLIYDASNRATGIPARFSGSGSG |
| | RAEDTAVYYCARDRGIAAGGNYYYYGMDVWGQGTTVTVSS | TDFTLTISSLEPEDFAVYYCQQRSNWPPFTEGPGT |
| | (SEQ ID NO: 369) | KVDIK (SEQ ID NO: 434) |
| HSV- | EVQLVESGGGLVQPGGSLRLSCAASGYVFTDYGMNWVRQAPG | DIQMTQSPSSLSASVGDRVTITCKASQNVGTNVA |
| AB16 | KGLEWMGWINTYIGEPIYADSVKGRFTFSLDTSKSTAYLQMNSL | WYQQKPGKAPKALIYSASFLYSGVPYRFSGSGS |
| | RAEDTAVYYCARGYRSYAMDYWGQGTLVTVSS (SEQ ID NO: | GTDFTLTISSLQPEDFATYYCQQYNIYPLTFGQGT |
| | 370) | KVEIK (SEQ ID NO: 435) |
| HSV- | EVQLVESGGGLVQPGGSLRLSCAASGFTFSSFGMHWVRQAPGK | DIQLTQSPSFESASVGDRVTITCKASQNVDTNVA |
| AB17 | GLEWVAYISSDSSAIYYADTVKGRFTISRDNAKNSLYLQMNSLR | WYQQKPGKAPKALIYSASYRYSGVPSRFSGSGS |
| | DEDTAVYYCGRGRENIYYGSRLDYWGQGTTVTVSS (SEQ ID | GTDFTLTISSLQPEDFATYYCQQYNNYPFTFGQG |
| | NO: 371) | TKLEIK (SEQ ID NO: 436) |
| HSV- | QVQLVESGGGVVQPGRSLRLSCAASGFTESSYTMHWVRQAPGK | EIVLTQSPGTLSLSPGERATLSCRASQSVGSSYLA |
| AB18 | GLEWVTFISYDGNNKYYADSVKGRFTISRDNSKNTLYLQMNSL | WYQQKPGQAPRLLIYGAFSRATGIPDRFSGSGSG |
| | RAEDTAIYYCARTGWLGPFDYWGQGTLVTVSS (SEQ ID NO: | TDFTLTISRLEPEDFAVYYCQQYGSSPWTFGQGT |
| | 372) | KVEIK (SEQ ID NO: 437) |
| HSV- | QVQLQESGPGLVRPSQTLSLTCTASGYTFTDYVIHWVKQPPGRG | DIQMTQSPSSLSASVGDRVTITCRASQNVGTAVA |
| AB19 | LEWIGYINPYDDDTTYNQKFKGRVTMLVDTSSNTAYLRLSSVT | WLQQTPGKAPKLLIYSASNRYTGVPSRFSGSGSG |
| | AEDTAVYYCARRGNSYDGYFDYSMDYWGSGTPVTVSS (SEQ | TDYTFTISSLQPEDIATYYCQQYTNYPMYTEGQG |
| | ID NO: 373) | TKVQIK (SEQ ID NO: 438) |
| HSV- | EVQLVESGGGLVQPGGSLRLSCAASGFTESDSWIHWVRQAPGK | DIQMTQSPSSLSASVGDRVTITCRASQDVSTAVA |
| AB20 | GLEWVAWISPYGGSTYYADSVKGRFTISADTSKNTAYLQMNSL | WYQQKPGKAPKWYSASFLYSGVPSRFSGSGSG |
| | RAEDTAVYYCARRHWPGGFDYWGQGTLVTVSS (SEQ ID NO: | TDFTLTISSLQPEDFATYYCQQYLYHPATFGQGT |
| | 374) | KVEIK (SEQ ID NO: 439) |
| HSV- | EVQLVESGGGLVQPGGSLRLSCAASGFTESRYWMSWVRQAPGK | EIVLTQSPGTLSLSPGERATLSCRASQRVSSSYLA |
| AB21 | GLEWVANIKQDGSEKYYVDSVKGRFTISRDNAKNSLYLQMNSL | WYQQKPGQAPRLLIYDASSRATGIPDRFSGSGSG |
| | RAEDTAVYYCAREGGWFGELAFDYWGQGTLVTVSS (SEQ ID | TDFTLTISRLEPEDFAVYYCQQYGSLPWTFGQGT |
| | NO: 375) | KVEIK (SEQ ID NO: 440) |
| HSV- | QVQLVESGGGVVQPGRSLRLDCKASGITFSNSGMHWVRQAPGK | EIVLTQSPATLSLSPGERATLSCRASQSVSSYLAW |
| AB22 | GLEWVAVIWYDGSKRYVADSVKGRFTISRDNSKNTLFLQMNSL | YQQKPGQAPRLLIYDASNRATGIPARFSGSGSGT |
| | RAEDTAVYYCATNDDYWGQGTLVTVSS (SEQ ID NO: 376) | DFTLTISSLEPEDFAVYYCQQSSNWPRTFGQGTK |
| | | VEIK (SEQ ID NO: 441) |
| HSV- | QVQLVQSGVEVKKPGASVKVSCKASGYTFTNYYMYWVRQAPG | EIVLTQSPATLSLSPGERATLSCRASKGVSTSGYS |
| AB23 | QGLEWMGGINPSNGGTNFNEKFKNRVTLTTDSSTTTAYMELKS | YLHWYQQKPGQAPRLLIYLASYLESGVPARFSG |
| | LQFDDTAVYYCARRDYRFDMGFDYWGQGTTVTVSS (SEQ ID | SGSGTDFTLTISSLEPEDFAVYYCQHSRDLPLTFG |
| | NO: 377) | GGTKVEIK (SEQ ID NO: 442) |
| HSV- | QVQLVESGGGVVQPGRSLRLSCAASGFTESVYGMNWVRQAPG | EIVLTQSPDFQSVTPKEKVTITCRASQSIGSSLHW |
| AB24 | KGLEWVAIIWYDGDNQYYADSVKGRFTISRDNSKNTLYLQMN | YQQKPDQSPKLLIKYASQSFSGVPSRFSGSGSGT |
| | GLRAEDTAVYYCARDLRTGPFDYWGQGTLVTVSS (SEQ ID NO: | DFTLTINSLEAEDAAAVYCHQSSSLPFTEGPGTK |
| | 378) | VDIK (SEQ ID NO: 443) |
| HSV- | EVQLVESGGGLVQPGGSLRLSCAASGYTFTSYNMHWVRQAPG | DIQMTQSPSSLSASVGDRVTITCRASSSVSYMHW |
| AB25 | KGLEWVGAIYPGNGDTSYNQKFKGRFTISVDKSKNTLYLQMNS | YQQKPGKAPKPLIYAPSNLASGVPSRFSGSGSGT |
| | LRAEDTAVYYCARVVYYSNSYWYFDVWGQGTLVTVSS (SEQ | DFTLTISSLQPEDFATYYCQQWSENPPTEGQGTK |
| | ID NO: 379) | VEIK (SEQ ID NO: 444) |
| HSV- | EVQLVESGGGLVQPGRSLRLSCAASGFTFNDYAMHWVRQAPG | EIVLTQSPATLSLSPGERATLSCRASQSVSSYLAW |
| AB26 | KGLEWVSTISWNSGSIGYADSVKGRFTISRDNAKKSLYLQMNSL | YQQKPGQAPRLLIYDASNRATGIPARFSGSGSGT |
| | RAEDTALYYCAKDIQYGNYYYGMDVWGQGTTVTVSS (SEQ ID | DFTLTISSLEPEDFAVYYCQQRSNWPITEGQGTRL |
| | NO: 380) | EIK (SEQ ID NO: 445) |
| HSV- | QAYLQQSGAELVRPGASVKMSCKASGYTFTSYNMHWVKQTPR | QIVLSQSPAILSASPGEKVTMTCRASSSVSYMHW |
| AB27 | QGLEWIGGIYPGNGDTSYNQKFKGKATLTVGKSSSTAYMQLSS | YQQKPGSSPKPWIYATSNLASGVPARFSGSGSGT |
| | LTSEDSAVYFCARYDYNYAMDYWGQGTSVTVSS (SEQ ID NO: | SYSFTISRVEAEDAATYYCQQWTFNPPTFGGGTR |
| | 381) | LEIK (SEQ ID NO: 446) |
| HSV- | QVQLQESGPGLVRPSQTLSLTCTVSGFTFTDFYMNWVRQPPGRG | DIQMTQSPSSLSASVGDRVTITCKASQNIDKYLN |
| AB28 | LEWIGFIRDKAKGYTTEYNPSVKGRVTMLVDTSKNQFSLRLSSV | WYQQKPGKAPKWYNTNNLQTGVPSRFSGSGS |
| | TAADTAVYYCAREGHTAAPFDYWGQGSLVTVSS (SEQ ID NO: | GTDFTFTISSLQPEDIATYYCLQHISRPRTFGQGT |
| | 382) | KVEIK (SEQ ID NO: 447) |
| HSV- | EVQLLESGGGLVQPGGSLRLSCAASGFTFSAYEMKWVRQAPGK | DIQMTQSPATLSLSPGERATLSCRASQSVSSYLA |
| AB29 | GLEWVSVIGPSGGFTFYADSVKGRFTISRDNSKNTLYLQMNSLR | WYQQKPGQAPRLLIYDASNRATGIPARFSGSGSG |
| | AEDTAVYYCATEGDNDAFDIWGQGTTVTVSS (SEQ ID | TDFTLTISSLEPEDFAVYYCQQRSNWPMYTEGQG |
| | NO: 383) | TKLEIK (SEQ ID NO: 448) |
| HSV- | QVQLVQSGAEVKKPGASVKVSCKASGFNIKDTYIHWVRQAPGQ | DIQMTQSPSSLSASVGDRVTITCKTSQDINKYMA |
| AB30 | RLEWMGRIDPANGYTKYDPKFQGRVTITADTSASTAYMELSSL | WYQQTPGKAPRLLIHYTSALQPGIPSRFSGSGSG |
| | RSEDTAVYYCAREGYYGNYGVYAMDYWGQGTLVTVSS (SEQ | RDYTFTISSLQPEDIATYYCLQYDNLWTFGQGTK |
| | ID NO: 384) | VEIK (SEQ ID NO: 449) |
| HSV- | QVQLVQSGAEVKKPGASVKVSCKASGYIFSNYWIQWVRQAPG | DIQMTQSPSSLSASVGDRVTITCGASENIYGALN |
| AB31 | QGLEWMGEILPGSGSTEYTENFKDRVTMTRDTSTSTVYMELSSL | WYQQKPGKAPKWYGATNLADGVPSRFSGSGS |
| | RSEDTAVYYCARYFFGSSPNWYFDVWGQGTLVTVSS (SEQ ID | GTDFTLTISSLQPEDFATYYCQNVLNTPLTFGQG |
| | NO: 385) | TKVEIK (SEQ ID NO: 450) |
| HSV- | QVQLQESGPGLVKPSETLSLTCAVSGHSISHDHAWSWVRQPPGE | DIQMTQSPSSLSASVGDSVTITCQASTDISSHLNW |
| AB32 | GLEWIGFISYSGITNYNPSLQGRVTISRDNSKNTLYLQMNSLRAE | YQQKPGKAPELLIYYGSHLLSGVPSRFSGSGSGT |
| | DTAVYYCARSLARTTAMDYWGEGTLVTVSS (SEQ ID | DFTFTISSLEAEDAATYYCGQGNRLPYTFGQGTK |
| | NO: 386) | VEIE (SEQ ID NO: 451) |
| HSV- | EVQLVQSGAEVKKPGESLKISCKGSGYIFTNYWIAWVRQMPGK | EIVLTQSPGTLSLSPGERATLSCRASQSVSSSFFA |
| AB33 | GLESMGIIYPGDSDIRYSPSFQGQVTISADKSITTAYLQWSSLKAS | WYQQKPGQAPRLLIYGASSRATGIPDRLSGSGSG |
| | DTAMYYCARHDIEGFDYWGRGTLVTVSS (SEQ ID NO: 387) | TDFTLTITRLEPEDFAVYYCQQYDSSAITFGQGTR |
| | | LEIK (SEQ ID NO: 452) |
| HSV- | QVQLQQSGAEVKKPGSSVRVSCKASGGTFNNNAINWVRQAPG | SSELTQDPAVSVALGQTVRVTCQGDSLRSYYAS |
| AB34 | QGLEWMGGIIPMFGTAKYSQNFQGRVAITADESTGTASMELSSL | WYQQKPGQAPVLVIYGKNNRPSGIPDRFSGSSSG |
| | RSEDTAVYYCARSRDLLLFPHHALSPWGRGTMVTVSS (SEQ ID | NTASLTITGAQAEDEADYYCSSRDSSGNHWVFG |
| | NO: 388) | GGTELTVL (SEQ ID NO: 453) |
| HSV- | QLQQSGTVLARPGASVKMSCKASGYSFTRYWMHWIKQRPGQG | QIVSTQSPAIMSASPGEKVTMTCSASSSRSYMQW |
| AB35 | LEWIGAIYPGNSDTSYNQKFEGKAKLTAVTSASTAYMELSSLTH | YQQKPGTSPKRWIYDTSKLASGVPARFSGSGSGT |
| | EDSAVYYCSRDYGYYFDFWGQGTTLTVSS (SEQ ID NO: 389) | SYSLTISSMEAEDAATYYCHQRSSYTFGGGTKLE |
| | | IK (SEQ ID NO: 454) |
| HSV- | QVQLVQSGAEVKKPGSSVKVSCKASGYTFTSYRMHWVRQAPG | DIQMTQSPSTESASVGDRVTITCSASSSISYMHW |
| AB36 | QGLEWIGYINPSTGYTEYNQKFKDKATITADESTNTAYMELSSL | YQQKPGKAPKLLIYTTSNLASGVPARFSGSGSGT |
| | RSEDTAVYYCARGGGVFDYWGQGTLVTVSS (SEQ ID | EFTLTISSLQPDDFATYYCHQRSTYPLTFGQGTK |
| | NO: 390) | VEVK (SEQ ID NO: 455) |
| HSV- | EVQLVESGGGLEQPGGSLRLSCAGSGFTFRDYAMTWVRQAPGK | DIVMTQSPLSLPVTPGEPASISCRSSQSLLYSIGYN |
| AB37 | GLEWVSSISGSGGNTYYADSVKGRFTISRDNSKNTLYLQMNSLR | YLDWYLQKSGQSPQLLIYLGSNRASGVPDRFSGS |
| | AEDTAVYYCAKDRLSITIRPRYYGLDVWGQGTTVTVSS (SEQ ID | GSGTDETLKISRVEAEDVGFYYCMQALQTPYTE |
| | NO: 391) | GQGTKLEIK (SEQ ID NO: 456) |
| HSV- | EVQLVESGGGLVQPGGSLRLSCAASGFTFNNYAMNWVRQAPG | DIVMTQSPDSLAVSLGERATINCKSSQSVLYRSN |
| AB38 | KGLDWVSTISGSGGTTNYADSVKGRFIISRDSSKHTLYLQMNSL | NRNFLGWYQQKPGQPPNLLIYWASTRESGVPDR |
| | RAEDTAVYYCAKDSNWGIVEDLWGRGTLVTVSS (SEQ ID NO: | ESGSGSGTDETLTISSLQAEDVAVYYCQQYYTTP |
| | 392) | YTEGQGTKLEIK (SEQ ID NO: 457) |
| HSV- | QVQLVQSGAEVAKPGTSVKLSCKASGYTFTDYWMQWVKQRPG | DIVMTQSHLSMSTSLGDPVSITCKASQDVSTVVA |
| AB39 | QGLEWIGTIYPGDGDTGYAQKFQGKATLTADKSSKTVYMHLSS | WYQQKPGQSPRRLIYSASYRYIGVPDRFTGSGAG |
| | LASEDSAVYYCARGDYYGSNSLDYWGQGTSVTVSS (SEQ ID | TDETETISSVQAEDLAVYYCQQHYSPPYTEGGGT |
| | NO: 393) | KLEIK (SEQ ID NO: 458) |
| HSV- | EVQLLESGGVLVQPGGSLRLSCAASGFTFSNFGMTWVRQAPGK | DIQMTQSPSSLSASVGDSITITCRASLSINTFLNW |
| AB40 | GLEWVSGISGGGRDTYFADSVKGRFTISRDNSKNTLYLQMNSLK | YQQKPGKAPNLLIYAASSLHGGVPSRFSGSGSGT |
| | GEDTAVYYCVKWGNIYFDYWGQGTLVTVSS (SEQ ID | DETLTIRTLQPEDEATYYCQQSSNTPETEGPGTVV |
| | NO: 394) | DFR (SEQ ID NO: 459) |
| HSV- | EVQLQESGGVLVKPGGSLKLSCAASGEVESSYDMSWVRQTPEK | DIQMTQSPASLSASVGETVTITCRASENIFSYLAW |
| AB41 | RLEWVAYISSGGGITYFPDTVQGRETVSRDNAKNTLYLQMNSL | YQQKQGKSPQLLVYNTKTLAEGVPSRFSGSGSG |
| | KSEDTAIYYCAAHVEGSSGPFAYWGQGTLVTVSA (SEQ ID NO: | TQFSLKINSLQPEDEGSYYCQHHYGTPETEGSGT |
| | 395) | KLEIK (SEQ ID NO: 460) |
| HSV- | QVQLVQSGAEVKKPGSSVKVSCKASGYTESSNVISWVRQAPGQ | ETVLTQSPGTLSLSPGERATLSCRASQSLGSSYLA |
| AB42 | GLEWMGGVIPIVDIANYAQRFKGRVTITADESTSTTYMELSSLRS | WYQQKPGQAPRLLIVGASSRAPGIPDRESGSGSG |
| | EDTAVYYCASTLGLVLDAMDYWGQGTLVTVSS (SEQ ID NO: | TDETLTISRLEPEDFAVYYCQQYADSPITEGQGTR |
| | 396) | LEIK (SEQ ID NO: 461) |
| HSV- | EVQLKESGPGLVAPGGSLSITCTVSGFSLTDSSINWVRQPPGKGL | DIVLTQSPASLAVSLGQRATISCRASESVDSYGQS |
| AB43 | EWLGMIWGDGRIDYADALKSRLSISKDSSKSQVFLEMTSLRTDD | YMHWYQQKAGQPPKWYLASNLESGVPARESG |
| | TATYYCARDGYFPYAMDFWGQGTSVTVSS (SEQ ID NO: 397) | SGSRTDETLTIDPVQAEDAATYYCQQNAEDSRTE |
| | | GGGTKLEIK (SEQ ID NO: 462) |
| HSV- | QVQLQQSGPELVKPGASVKISCKASGYSFTSYWIHWIKQRPGQG | DIQMTQSPASLSVSVGDTITLTCHASQNIDVWLS |
| AB44 | LEWIGMIDPSDGETRLNQRFQGRATLTVDESTSTAYM | QLRSPTSWEQQKPGNIPKWYKASNLHTGVPSRFSGSGSG |
| | EDSAVYYCTRLKEYGNYDSEYEDVWGAGTLVTVSS (SEQ ID | TGETLTISSLQPEDIATYYCQQAHSYPETEGGGTK |
| | NO: 398) | LEIK (SEQ ID NO: 463) |
| HSV- | QAQLQVSGAEVVKPGASVKMSCKASGYTFTSYNMHWVKQTPG | EIVLTQSPATMSASPGERVTITCSAHSSVSFMHW |
| AB45 | QGLEWIGYIYPGNGATNYNQKFQGKATLTADTSSSTAYMQISSL | FQQKPGTSPKLWIYSTSSLASGVPARFGGSGSGT |
| | TSEDSAVYFCARGDSVPFAYWGQGTLVTVSA (SEQ ID | SYSLTISSMEAEDAATYYCQQRSSFPLTFGAGTK |
| | NO: 399) | LELK (SEQ ID NO: 464) |
| HSV- | QAQLQVSGAEVVKPGASVKMSCKASGYTFTSYNMHWVKQTPG | EIVLTQSPATMSASPGERVTITCSAHSSVSFMHW |
| AB46 | QGLEWIGYIYPGNGATNYNQKFQGKATLTADPSSSTAYMQISSL | FQQKPGTSPKLWIYSTSSLASGVPARFGGSGSGT |
| | TSEDSAVYFCARGDSVPFAYWGQGTLVTVSA (SEQ ID | SYSLTISSMEAEDAATYYCQQRSSFPLTFGAGTK |
| | NO: 400) | LELK (SEQ ID NO: 464) |
| HSV- | QVQLVESGGGVVQPGRSLRLSCVASGETESSYGMHWVRQAPGK | EIVLTQSPATLSLSPGERTTLSCRASQRISTYLAW |
| AB47 | GLEWVAIIWYDGSNKYYADSVKGRETISRDNSKNTQYLQMNSL | YQQKPGQAPRLLIYDASKRATGIPARFSGSGSGT |
| | RAEDTAVYYCASVATSGDFDYYGMDVWGQGTTVTVSS (SEQ | GETLTISSLEPEDFAVYYCQQRSNWPLTEGGGTK |
| | ID NO: 401) | VEIK (SEQ ID NO: 465) |
| HSV- | EVQLVESGGGVVRPGGSLRLSCAASGETFDDYGMTWVRQAPG | DIQMTQSPSSLSASLGDRVTITCRASQSINSYLNW |
| AB48 | RGLEWVSGIHWHGKRTGYADSVKGRFTISRDNAKKSLYLQMNS | YQQKPGKAPKLLIYVASSLQSGVPSRFSGSGSGT |
| | LKGEDTALYHCVRGGMSTGDWFDPWGQGTLVIVSS (SEQ ID | EFTLTISNLQPEDEATYYCQQSYSTPPITEGQGTR |
| | NO: 402) | LEIK (SEQ ID NO: 466) |
| HSV- | EVMLVESGGALVKPGGSLKLSCAASGETESNYAMSWVRQIPEK | QIVLTQSPAIMSASLGERVTMTCTASSSVSSSYLH |
| AB49 | RLEWVATISSGGSHTYYLDSVKGRFTISRDNARDTLYLQMSSLR | WYQQKPGSSPKLWIYSTSNLASGVPARFSGSGSG |
| | SEDTALYYCARLFTGYAMDYWGQGTSVTVSS (SEQ ID NO: | TEYSLTISSMEAEDDATYYCHQYYRLPPITEGAG |
| | 403) | TKLELK (SEQ ID NO: 467) |
| HSV- | EVQLVESGGNLEQPGGSLRLSCTASGETESRSAMNWVRRAPGK | DIQMTQSPSSVSASVGDRVTITCRASQGIFSWLA |
| AB50 | GLEWVSGISGSGGRTYYADSVKGRFTISRDNSKNTLYLQMNSLS | WYQQKPGKAPKLLIYAASSLQSGVPSRESGSGSG |
| | AEDTAAYYCAKDSYTTSWYGGMDVWGHGTTVTVSS (SEQ ID | TDETLTISSLQPEDFAIYYCQQANSVPITEGQGTR |
| | NO: 404) | LEIK (SEQ ID NO: 468) |
| HSV- | QVQLVQSGAEVKKPGSSVKVSCKASGYIFTNYNIHWVKKSPGQ | DIQMTQSPSSLSASVGDRVTITCKASQDIDRYMA |
| AB51 | GLEWIGAIYPGNGDAPYSQKFQGKATLTADTSTSTTYMELSSLR | WYQDKPGKAPRLLIHDTSTLQSGVPSRFSGSGSG |
| | SEDTAVYYCVRANWDVAFAYWGQGTLVTVSS (SEQ ID NO: | RDYTLTISNLEPEDEATYYCLQYDNLWTEGGGT |
| | 405) | KVEIK (SEQ ID NO: 469) |
| HSV- | EVQLVESGGGVIQPGGSLRLSCAASGETEDDYAMNWVRQGPGK | DIQMTQSPSTLSASVGDRVTITCRASQSIRSWLA |
| AB52 | GLEWVSAISGDGGSTYYADSVKGRFTISRDNSKNSLYLQMNSLR | WYQQKPGKAPKLLIYKASSLESGVPSRFSGSGSG |
| | AEDTAFFYCAKDLRNTIEGVVIPDAFDIWGQGTMVTVSS (SEQ | TEETLTISSLQPDDEATYYCQQYNSYSYTEGQGT |
| | ID NO: 406) | KLEIK (SEQ ID NO: 470) |
| HSV- | QVQLVQSGAEVKKPGASVKVSCKVSGETLTELSIHWVRQAPGK | DIQMTQSPSSLSASAGDRVTITCRASQAIRNDLG |
| AB53 | GLEWMGGFDPEDGETIYAQKFQGRVTMTEDTSTDTAYMELTSL | WYQQKPGKAPKRLIYAAFNLQSGVPSRESGSGS |
| | RSEDTAVYYCSTIEGVVTNEDNWGQGTLVTVSS (SEQ ID NO: | GTEETLTISSLQPEDLASYYCQQYNRYPWTEGQG |
| | 407) | TKVEIK (SEQ ID NO: 471) |
| HSV- | QVQLQESGPGLVKPSETLSLTCTVSGGSESSHEWSWIRQPPGKGL | EIVLTQSPGTLSLSPGERATLSCRASQSVSSSYLA |
| AB54 | EWIGYILYTGGTSENPSLKSRVSMSVGTSKNQESLKLSSVTAADT | WYQQKPGQAPRLLIVGASSRATGIPDRESGSGSG |
| | AVYYCARARSGITFTGIIVPGSFDIWGQGTMVTVSS (SEQ | TDETLTISRLEPEDFAVYYCQQYGSSPWTEGQGT |
| | ID NO: 408) | KVEIK (SEQ ID NO: 472) |
| HSV- | QVQLVESGGGVVQPGKSLRLSCVASGFTFSSDAMYWVRQAPG | DIQMTQSPSSLSASVGDRVTITCRASQSISSYLNW |
| AB55 | KGLEWVAVIYYDGNYQYYEDSVKGRFTISRDNSQNTLDLQMNS | YQQKPGKAPKLLIYAASSLQSGVPSRFSGSGSGT |
| | LRVDDTAVYFCARLNWDYWYLDLWGRGTLVTVSS (SEQ ID | DFTLTISSLQPEDFATYYCQQSYSTPPITFGQGTR |
| | NO: 409) | LEIK (SEQ ID NO: 473) |
| HSV- | EVQLVESGGGLVQPGRSLRLSCAASGFTFDDYTMHWVRQAPGK | AEIVMTQSPATLSVSPGERATLSCRASQSVSSNL |
| AB56 | GLEWVSGISWNSGSIGYADSVKGRFTISRDNAKKSLYLQMNSLR | AWYQQKPGQAPRLLIYGASTRATGIPARFSGSGS |
| | AEDTALYYCAKDNSGYGHYYYGMDVWGQGTTVTVAS (SEQ | GTEFTLTISSLQSEDFAVYYCQHYINWPLTFOGG |
| | ID NO: 410) | TKVEIK (SEQ ID NO: 474) |
| HSV- | EVQLVESGGGLVQPGRSLRLSCVASGFTFNDYAMHWVRQAPG | AEIVMTQSPATLSVSPGERATLSCRASQSVSSNL |
| AB 57 | KGLEWVSVISWNSDSIGYADSVKGRFTISRDNAKNSLYLQMHSL | AWYQQKPGQAPRLLIYGASTRATGIPARFSGSGS |
| | RAEDTALYYCAKDNHYGSGSYYYYQYGMDVWGQGTTVTVSS | GTEFTLTISSLQSEDFAVYYCQHYINWPLTFOGG |
| | (SEQ ID NO: 411) | TKVEIK (SEQ ID NO: 474) |
| HSV- | EVQLVESGGGLVKPGGSLRLSCAASGFTERNYNINWVRQAPGK | DIQVTQSPSPLSASVGDRVTITCRASQGISNYLA |
| AB58 | GLEWVSLISGSSSYIYYADSVKGRFTVSRDNAKNSLYLQMNSLR | WYQQKPGRVPQLLIYAASTLQSGVPSRFSGSGSG |
| | AEDTAVYYCARRTLSYYVMDVWGQGTTVTVSS (SEQ ID NO: | TDFTLTISSLQPEDVATYYCQKYNSAPYTEGQGT |
| | 412) | KLEIK (SEQ ID NO: 475) |
| HSV- | QVQLQESGPGLVKPSETLSLTCTVSGDSVSSSYWTWIRQPPGKG | AIQMTQSPSSLSASVGDRVTITCRASQGIRNDLG |
| AB59 | LEWIGYIYYSGSSNYNPSLKSRATISVDTSKNQFSLKLSSVTAAD | WYQQKPGKAPKLLIYAAS SLQSGVPSRFAGRGS |
| | TAVYYCAREGNVDTTMIFDYWGQGTLVTVSS (SEQ ID | GTDFTLTISSLQPEDFATYYCLQDFNYPWTFGQG |
| | NO: 413) | TKVEIK (SEQ ID NO: 476) |
| HSV- | QVQLVESGGGVVQPGRSLRLSCAASGFTFSSFGMHWVRQAPGK | QSVLTQPPSVSAAPGQKVTISCSGSSSNIGNNYVS |
| AB60 | GLEWVAVISFDGSIKYSVDSVKGRFTISRDNSKNTLFLQMNSLR | WYQQLPGTAPKWYDNNKRPSGIPDRFSGSKSG |
| | AEDTAVYYCARDRLNYYDSSGYYHYKYYGMAVWGQGTTVTV | TSTTLGITGLQTGDEADYYCGTWDSRLSAVVFG |
| | SS (SEQ ID NO: 414) | GGTKLTVL (SEQ ID NO: 477) |
| HSV- | QVQLVQSGAEVKKPGASVKVSCKASGYTFTSSYINWVRQAPGQ | QSALTQPASVSGSPGQSITISCTGTSSDVGSYNYV |
| AB61 | GLEWMGTINPVSGSTSYAQKFQGRVTMTRDTSISTAYMELSRLR | NWYQQHPGKAPKLMIYGVSKRPSGVSNRFSGSK |
| | SDDTAVYYCARGGWFDYWGQGTLVTVSS (SEQ ID NO: 415) | SGNTASLTISGLQAEDEADYYCGTFAGGSYYGV |
| | | FOGGTKLTVL (SEQ ID NO: 478) |
| HSV- | EVQLVESGGGLVQPGGSLRLSCAASGYDFTHYGMNWVRQAPG | DIQLTQSPSSLSASVGDRVTITCSASQDISNYLNW |
| AB62 | KGLEWVGWINTYTGEPTYAADFKRRFTFSLDTSKSTAYLQMNS | YQQKPGKAPKVLIYFTSSLHSGVPSRFSGSGSGT |
| | LRAEDTAVYYCAKYPYYYGTSHWYFDVWGQGTLVTVSS (SEQ | DFTLTISSLQPEDFATYYCQQYSTVPWTFGQGTK |
| | ID NO: 416) | VEIK (SEQ ID NO: 479) |
| HSV- | QVQLVQSGAEVMKPGSSVKVSCKASGYTFSWYWLEWVRQAPG | EIVMTQSPATLSVSPGERATLSCRASQSIGTNIHW |
| AB63 | HGLEWMGEIDPGTFITNYNEKFKARVTFTADTSTSTAYMELSSL | YQQKPGQAPRLLIYYASESISGIPARFSGSGSGTE |
| | RSEDTAVYYCARFSHFSGSNYDYFDYWGQGTLVTVSS (SEQ ID | FTLTISSLQSEDFAVYYCQQSWSWPTTFGGGTKV |
| | NO: 417) | EIK (SEQ ID NO: 480) |
| HSV- | EVQLVESGGGLVQPGGSLRLSCAVSGYSITSGYSWNWIRQAPGK | DIQLTQSPSSLSASVGDRVTITCRASQSVDYDGD |
| AB64 | GLEWVASITYDGSTNYADSVKGRFTISRDDSKNTFYLQMNSLRA | SYMNWYQQKPGKAPKLLIYAASYLESGVPSRFS |
| | EDTAVYYCARGSHYFGHWHFAVWGQGTLVTVSS (SEQ ID NO: | GSGSGTDFTLTISSLQPEDFATYYCQQSHEDPYTF |
| 418) | GQGTKVEIK (SEQ ID NO: 481) | |
| HSV- | QVQLVESGGGVVQPGRSLRLSCAASGFTESSYGMHWVRQAPGK | DIVMTQSPLSLTVTPGEPASISCRSSQSLLYSNGY |
| AB65 | GLEWVAVISYEESNRYHADSVKGRFTISRDNSKITLYLQMNSLR | NYLDWYLQKPGQSPQVLISLGSNRASGVPDRFS |
| | TEDTAVYYCARDGGIAAPGPDYWGQGTLVTVSS (SEQ ID NO: | GSGSGTDFTLKISRVEAEDVGVYYCMQARQTPF |
| | 419) | TFGPGTKVDIR (SEQ ID NO: 482) |
| HSV- | EVQLQQSGPELVKPGASVRMSCKASGYTFTDYNIHWVKQSHGK | NIVLTQSPASLAVSLGQRATISCRASESVDNYGH |
| AB66 | SLEWIGYIYPNNGDNGYNQKFRGKATLTVDKSSSTAYMELRSLT | SFMHWYQQKPGQPPKWYLASNLESGVPARFS |
| | SDDSAVYYCARGRLRYFDVWGTGTTVTVSS (SEQ ID | GSGSRTDFTLTLDPVEADDAATYYCQQYNEDPP |
| | NO: 614) | TFGSGTKLEIK (SEQ ID NO: 866) |
| HSV- | EVQLVESGGDLVQPGGSLRLSCAASGFTESSYWMSWVRQAPGK | AIQLTQSPSSLSASVGDRVTITCRPSQGINWELA |
| AB67 | GLEWVANIKQDGSEKYYVDSVKGRFTISRDNAKNSLYLQMNSL | WYQQKPGKAPKLLIYDAS SLEQGVPSRFSGSGSG |
| | RAEDTAVYYCARDRGSLYYWGQGTLVTVSS (SEQ ID | TDFTLTISSLQPEDFATYYCQQFNSYPLTEGGGTK |
| | NO: 615) | VEIK (SEQ ID NO: 867) |
| HSV- | QVQLVQSGAEVKKPGSSVKVSCKASEGTFSSYAMSWVRQAPG | SYVLTQPPSASGTPGQSVTISCSGSTSNIGSHYVV |
| AB68 | QGLEWMGGIIPIEGTVNYAQKFQGRVTMTRDTSTSTVYMELSSL | WYQQLPGTAPRLLIYRNHQRPSGVPDRLSGSKS |
| | RSDDTAVYYCARRRGAKFDYWGQGTLVTVSS (SEQ ID NO: | GTSASLAIGGLRSEDEADYYCAVWDDTLSGWVF |
| | 616) | GGGTKLTVL (SEQ ID NO: 868) |
| HSV- | EVQLVQSGAEVKKPGESLKISCKGSGYSFTSYWIGWVRQMPGK | EIVLTQSPGTLSLSPGERATLSCRASQSISSSYLA |
| AB69 | GLEWMGVIYPGDSYTRYSPSFQGQVTISADKSISTAYLQWSSLK | WYQQKPGQAPRLLIYGASSRATGIPDRFSGSGSG |
| | ASDTAMYYCARMPNWGSLDHWGQGTLVTVSS (SEQ ID NO: | TDFTLTISRLEPEDFAVYYCQQYASFGQGTKVEI |
| | 1117) | K (SEQ ID NO: 1118) |

In some embodiments, one or more polynucleotides of the present disclosure encode an antibody comprising a heavy chain variable region comprising the sequence of SEQ ID NO: 355, and light chain variable region comprising the sequence of SEQ ID NO: 420. In some embodiments, one or more polynucleotides of the present disclosure encode an antibody comprising a heavy chain variable region comprising the sequence of SEQ ID NO: 356, and light chain variable region comprising the sequence of SEQ ID NO: 421. In some embodiments, one or more polynucleotides of the present disclosure encode an antibody comprising a heavy chain variable region comprising the sequence of SEQ ID NO: 357, and light chain variable region comprising the sequence of SEQ ID NO: 422.

A polynucleotide of the present disclosure encoding an antibody may further encode additional coding and non-coding sequences. Examples of additional coding and non-coding sequences may include, but are not limited to, sequences encoding additional polypeptide tags (e.g., encoded in-frame with the antibody in order to produce a fusion protein), introns (e.g., native, modified, or heterologous introns), 5′ and/or 3′ UTRs (e.g., native, modified, or heterologous 5′ and/or 3′ UTRs), and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., green fluorescent protein, red fluorescent protein, etc.) and tags that have a detectable enzymatic activity (e.g., alkaline phosphatase, etc.), tags containing secretory sequences, signal sequences, leader sequences, and/or stabilizing sequences, protease cleavage sites (e.g., furin cleavage sites, TEV cleavage sites, Thrombin cleavage sites, etc.), and the like. In some embodiments, the 5′ and/or 3′UTRs increase the stability, localization, and/or translational efficiency of the polynucleotides. In some embodiments, the 5′ and/or 3′UTRs improve the level and/or duration of protein expression. In some embodiments, the 5′ and/or 3′UTRs include elements (e.g., one or more miRNA binding sites, etc.) that may block or reduce off-target expression (e.g., inhibiting expression in specific cell types (e.g., neuronal cells), at specific times in the cell cycle, at specific developmental stages, etc.). In some embodiments, the 5′ and/or 3′UTRs include elements (e.g., one or more miRNA binding sites, etc.) that may enhance antibody expression in specific cell types.

In some embodiments, a polynucleotide of the present disclosure encodes a leader, signal, and/or secretory sequence (in-frame) at the N-terminus of an encoded antibody. Any leader, signal, and/or secretory sequence known in the art may be encoded by a polynucleotide of the present disclosure, including, for example, a native antibody signal sequence (e.g., any of the antibody leader sequences described in Retter I et al. VBASE2, an integrative V gene database. Nucleic Acids Res. 2005 Jan. 1; 33: D671-4), or a heterologous or synthetic signal sequence (see e.g., von Heijne G. (1983) Patterns of amino acids near signal-sequence cleavage sites. Eur J Biochem 133 (1) 17-21; Martoglio B. and Dobberstein B. (1998) Signal sequences: More than just greasy peptides. Trends Cell Biol 8 (10), 410-5; Hegde R. S. and Bernstein H. D. (2006) The surprising complexity of signal sequences. Trends Biochem Sci 31(10), 563-71; Kapp K., Schrempf S., Lemberg M. K. and Dobberstein B. (2009) Post-Targeting Functions of Signal Peptides. Chapter in: Protein Transport into the Endoplasmic Reticulum, Landes Bioscience; and the sequences disclosed in www.signalpeptide.de). Exemplary secretion sequences include the human CD33 leader sequence (MPLLLLLPLLWAGALA, SEQ ID NO: 483), the human IL2 leader sequence (MYRMQLLSCIALSLALVTNS, SEQ ID NO: 484), the human tissue plasminogen activator leader sequence (MDAMKRGLCCVLLLCGAVFVSP, SEQ ID NO: 485), human antibody leader sequences (Tables 3A-C), and the synthetic secrecon leader sequence (MWWRLWWLLLLLLLLWPMVWA, SEQ ID NO: 486).

TABLE 3A
Human antibody heavy chain leader sequences

| | SEQ |
|---|---|
| Leader sequence: | ID NO: |
| MDWTWRILFLVAAATGAHS | 487 |
| MDWTWRVFCLLAVAPGAHS | 488 |
| MEFGLSWVFLVAIIKGVQC | 489 |
| MEFGLSWVFLVALLRGVQC | 490 |
| MEFGLSWVFLVAILKGVQC | 491 |
| MSVSFLIFLPVLGLPWGVLS | 492 |
| MDWTWSILFLVAAPTGAHS | 493 |
| MEFWLSWVFLVAILKGVQC | 494 |
| MDWTWRILFLVAAATSAHS | 495 |
| MDWIWRILFLVGAATGAHS | 496 |
| MELGLSWVFLVAILEGVQC | 497 |
| MELGLCWVFLVAILEGVQC | 498 |
| MKHLWFFLLLVAAPRWVLP | 499 |
| MKHLWFLLLWCQLPDVGVLS | 500 |
| MDCTWRILFLVAAATGTHA | 501 |
| MDTLCYTLLLLTTPSWVLS | 502 |
| MELGLRWVFLVAILEGVQC | 503 |
| MTEFGLSWVFLVAIFKGVQC | 504 |
| MGSTAILALLLAVLQGVCS | 505 |
| MDTLCSTLLLLTIPSWVLS | 506 |
| MEFGLSWIFLAAILKGVQC | 507 |
| MDWTWRILFLVAAATDAYS | 508 |
| MELGLSWIFLLAILKGVQC | 509 |
| MEFGLSWLFLVAILKGVQC | 510 |
| MEFGLSWVFLVVILQGVQC | 511 |
| MGSTAILGLLLAVLQGVCA | 512 |
| MKHLWFFLLLVAAPRWVLS | 513 |

TABLE 3B
Human antibody light chain (kappa)
leader sequences

| Leader sequence: | SEQ ID NO: |
|---|---|
| MDMRVPAQLLGLLLLWLRGARC | 514 |
| MDMRVPAQLLGLLQLWLSGARC | 515 |
| MDMRVPAQLLGLLLLWLSGARC | 516 |
| MDMRVPAQLLGLLLLWLPDTRC | 517 |
| MDMRVPAQLLGLLLLWFPGARC | 518 |
| MDMRVLAQLLGLLLLCFPGARC | 519 |
| MDMRVPAQLLGLLLLWFPGSRC | 520 |
| MDMRVPAQRLGLLLLWFPGARC | 521 |
| MRVPAQLLGLLLLWLPGARC | 522 |
| MDMRVPAQLLGLLLLWLPGARC | 523 |
| MDMRVPAQLLGLLLLWLPGAKC | 524 |
| MRLPAQLLGLLMLWVPGSSE | 525 |
| MVSPLQFLRLLLLWVPASRG | 526 |
| MRLPAQLLGLLMLWVPGSSE | 527 |
| MRLPAQLLGLLMLWVPGSSG | 528 |
| MRLPAQLLGLLMLWIPGSSA | 529 |
| MRLPAQLLGLLMLWVSGSSG | 530 |
| MRLLAQLLGLLMLWVPGSSG | 531 |
| METPAQLLFLLLLWLPDTTG | 532 |
| MEAPAQLLFLLLLWLPDTTG | 533 |
| MEAPAQLLFLLLLWLTDTTG | 534 |
| MEPWKPQHSFFFLLLLWLPDTTG | 535 |
| MVLQTQVFISLLLWISGAYG | 536 |
| MGSQVHLLSFLLLWISDTRA | 537 |
| MLPSQLIGFLLLWVPASRG | 538 |

TABLE 3C
Human antibody light chain (lambda)
leader sequences

| Leader sequence: | SEQ ID NO: |
|---|---|
| MAWSPLFLTLITHCAGSWA | 539 |
| MAWSPLLLTLLAHCTGSWA | 540 |
| MASFPLLLTLLTHCAGSWA | 541 |
| MAGFPLLLTLLTHCAGSWA | 542 |
| MTCSPLLLTLLIHCTGSWA | 543 |
| MAWALLLLSLLTQGTGSWA | 544 |
| MAWALLLLTLLTQGTGSWA | 545 |
| MAWALLLLTLLTQDTGSWA | 546 |
| MAWIPLFLGVLAYCTGSVA | 547 |
| MAWTALLLSLLAHFTGSVA | 548 |
| MAWTPLLLPLLTFCTVSEA | 549 |
| MAWTPLWLTLLTLCIGSVV | 550 |
| MAWTVLLLGLLSHCTGSVT | 551 |
| MAWATLLLPLLNLYTGSIA | 552 |
| MAWIPLLLPLLTLCTGSEA | 553 |
| MAWIPLLLPLLILCTVSVA | 554 |
| MAWVSFYLLPFIFSTGLCA | 555 |
| MAWTQLLLLFPLLLHWTGSLS | 556 |
| MAWTPLLFLTLLLHCTGSLS | 557 |
| MAWTPLLLLLLSHCTGSLS | 558 |
| MAWTPLLLLFLSHCTGSLS | 559 |
| MAWTLLLLVLLSHCTGSLS | 560 |
| MAWAPLLLTLLAHCTGSWA | 561 |
| MAWTPLFLFLLTCCPGSNS | 562 |
| MAWMMLLLGLLAYGSGVDS | 563 |
| MAWAPLLLTLLSLLTGSLS | 564 |
| MPWALLLLTLLTHSAVSVV | 565 |

In some embodiments, a polynucleotide of the present disclosure encoding an antibody is operably linked to one or more (e.g., one or more, two or more, three or more, four or more, five or more, ten or more, etc.) regulatory sequences. The term “regulatory sequence” may include enhancers, insulators, promoters, and other expression control elements (e.g., polyadenylation signals). Any suitable enhancer(s) known in the art may be used, including, for example, enhancer sequences from mammalian genes (such as globin, elastase, albumin, α-fetoprotein, insulin and the like), enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like), and any combinations thereof. Any suitable insulator(s) known in the art may be used, including, for example, HSV chromatin boundary (CTRL/CTCF-binding/insulator) elements CTRL1 and/or CTRL2, chicken hypersensitive site 4 insulator (cHS4), human HNRPA2B1-CBX3 ubiquitous chromatin opening element (UCOE), the scaffold/matrix attachment region (S/MAR) from the human interferon beta gene (IFNB1), and any combinations thereof. Any suitable promoter (e.g., suitable for transcription in mammalian host cells) known in the art may be used, including, for example, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), promoters from heterologous mammalian genes (such as the actin promoter (e.g., the β-actin promoter, a ubiquitin promoter (e.g., a ubiquitin C (UbC) promoter), a phosphoglycerate kinase (PG) promoter, an immunoglobulin promoter, from heat-shock promoters, and the like), promoters from homologous mammalian genes (e.g., native human immunoglobulin promoters), synthetic promoters (such as the CAGG promoter), and any combinations thereof, provided such promoters are compatible with the host cells. Regulatory sequences may include those which direct constitutive expression of a nucleic acid, as well as tissue-specific regulatory and/or inducible or repressible sequences.

In some embodiments, a polynucleotide of the present disclosure is operably linked to one or more heterologous promoters. In some embodiments, the one or more heterologous promoters are one or more of constitutive promoters, tissue-specific promoters, temporal promoters, spatial promoters, inducible promoters and repressible promoters. In some embodiments, the one or more heterologous promoters are one or more of the human cytomegalovirus (HCMV) immediate early promoter, the human elongation factor-1 (EF1) promoter, the human β-actin promoter, the human UbC promoter, the human PGK promoter, a human immunoglobulin promoter, the synthetic CAGG promoter, and any combinations thereof. In some embodiments, a polynucleotide of the present disclosure encoding an antibody is operably linked to an HCMV promoter.

In some embodiments, a polynucleotide of the present disclosure does not comprise the coding sequence of (e.g., a transgene encoding) a Collagen alpha-1 (VII) chain polypeptide (COLT). In some embodiments, a polynucleotide of the present disclosure does not comprise the coding sequence of (e.g., a transgene encoding) a Lysyl hydroxylase 3 polypeptide (LH3). In some embodiments, a polynucleotide of the present disclosure does not comprise the coding sequence of (e.g., a transgene encoding) a Keratin type I cytoskeletal 17 polypeptide (KRT17). In some embodiments, a polynucleotide of the present disclosure does not comprise the coding sequence of (e.g., a transgene encoding) a transglutaminase (TGM) polypeptide (e.g., a human transglutaminase polypeptide such as a human TGM1 polypeptide). In some embodiments, a polynucleotide of the present disclosure does not comprise the coding sequence of (e.g., a transgene encoding) a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, a transglutaminase (TGM) polypeptide (e.g., a human transglutaminase polypeptide such as a human TGM1 polypeptide), a cosmetic protein, and/or any chimeric polypeptides thereof.

Polynucleotides Encoding Single-chain Antibodies

In some embodiments, a recombinant nucleic acid (e.g., a recombinant herpes virus genome) of the present disclosure comprises one or more polynucleotides encoding an antibody, where the antibody is a single-chain antibody. Any suitable form of single-chain antibody known in the art may be encoded by a polynucleotide of the present disclosure. In some embodiments, the single-chain antibody comprises a heavy chain variable region and a light chain variable region. In some embodiments, the single-chain antibody comprises a heavy chain variable region and a light chain variable region separated by a linker polypeptide. In some embodiments, the single-chain antibody comprises, from N-terminus to C-terminus, 1) a heavy chain variable region, 2) a linker polypeptide, and 3) a light chain variable region. In some embodiments, the single-chain antibody comprises, from N-terminus to C-terminus, 1) a light chain variable region, 2) a linker polypeptide, and 3) a heavy chain variable region. In some embodiments, the single-chain antibody further comprises an antibody hinge region (e.g., an IgG1 hinge region). An exemplary IgG1 hinge region is provided as SEQ ID NO: 603. In some embodiments, the single-chain antibody further comprises an antibody Fc region (e.g., an IgG1 Fc region). An exemplary human IgG1 Fc region is provided as SEQ ID NO: 604. In some embodiments, the single-chain antibody is an scFv-Fc antibody (e.g., an scFv fused to the hinge and Fc region of an IgG1 antibody heavy chain).

Any suitable linker polypeptide known in the art may be used in a single-chain antibody of the present disclosure, including, for example, a GGGGSGGGGSGGGGS (SEQ ID NO: 566) linker, a GGSSRSSSSGGGGSGGGG (SEQ ID NO: 567) linker, a GGGGSGGGGSGGGGSGGGGS (SEQ ID NO: 568) linker, a CGGGSGGGGSGGGGS (SEQ ID NO: 569) linker, a SHGGHGGGGSGGGGS (SEQ ID NO: 570) linker, a MGGMSGGGGSGGGGS (SEQ ID NO: 571) linker, a YGGYSGGGGSGGGGS (SEQ ID NO: 572) linker, a WGGYSGGGGSGGGGS (SEQ ID NO: 573) linker, a SVSVGMKPSPRP (SEQ ID NO: 574) linker, a VISNHAGSSRRL (SEQ ID NO: 575) linker, a PWIPTPRPTFTG (SEQ ID NO: 576) linker, a RGRGRGRGRGR (SEQ ID NO: 577) linker, etc.

Exemplary polynucleotides encoding single-chain antibodies comprising a leader sequence, an antibody heavy chain variable region, a linker polypeptide, an antibody light chain variable region, and an antibody hinge and Fc region are provided as SEQ ID NOS: 578-583.

Polynucleotides Comprising Multiple Expression Cassettes

In some embodiments, a recombinant nucleic acid (e.g., recombinant herpes virus genome) of the present disclosure comprises one or more polynucleotides encoding an antibody, where at least one of the polynucleotides comprises two or more expression cassettes. In some embodiments, the polynucleotide comprises, from 5′ to 3′, a first expression cassette encoding a polypeptide comprising an antibody heavy chain variable region (e.g., a full-length antibody heavy chain), and a second expression cassette encoding a polypeptide comprising an antibody light chain variable region (e.g., a full-length antibody light chain). In some embodiments, the polynucleotide comprises, from 5′ to 3′, a first expression cassette encoding a polypeptide comprising an antibody light chain variable region (e.g., a full-length antibody light chain), and a second expression cassette encoding a polypeptide comprising an antibody heavy chain variable region (e.g., a full-length antibody heavy chain). In some embodiments, the first and second expression cassettes have independent regulatory sequences (e.g., promoters, enhancers, polyadenylation signals, etc.).

In some embodiments, the first and second expression cassettes are in the same orientation in the DNA. In some embodiments, the first and second expression cassettes are in opposite orientations to one another in the DNA. Without wishing to be bound by theory, incorporating two expression cassettes in an antisense orientation (opposite strands of DNA) may help to avoid read-through and ensure proper expression of each cassette.

Polynucleotides Encoding Polycistronic mRNAs

In some embodiments, a recombinant nucleic acid (e.g., recombinant herpes virus genome) of the present disclosure comprises one or more polynucleotides encoding an antibody, where at least one of the polynucleotides encodes a polycistronic mRNA. In some embodiments, the polycistronic mRNA comprises: 1) a first open reading frame (ORF) encoding a polypeptide comprising an antibody heavy chain variable region (e.g., an antibody heavy chain), and 2) a second open reading frame (ORF) encoding a polypeptide comprising an antibody light chain variable region (e.g., an antibody light chain). In some embodiments, the polycistronic mRNA comprises: 1) a first open reading frame (ORF) encoding a polypeptide comprising an antibody light chain variable region (e.g., an antibody light chain), and 2) a second open reading frame (ORF) encoding a polypeptide comprising an antibody heavy chain variable region (e.g., an antibody heavy chain). In some embodiments, the polycistronic mRNA further comprises an internal ribosomal entry site (IRES) separating the first ORF and the second ORF. In some embodiments, the polycistronic mRNA comprises, from 5′ to 3′, the first ORF encoding the polypeptide comprising the antibody heavy chain variable region—the IRES—the second ORF encoding the polypeptide comprising the antibody light chain variable region. In some embodiments, the polycistronic mRNA comprises, from 5′ to 3′, the first ORF encoding the polypeptide comprising the antibody light chain variable region—the IRES—the second ORF encoding the polypeptide comprising the antibody heavy chain variable region.

Any suitable IRES known in the art may be used in the polycistronic mRNAs of the present disclosure, including, for example, a virally-derived IRES (e.g. an IRES derived from a poliovirus, rhinovirus, encephalomyocarditis virus (EMCV), foot-and-mouth disease virus, hepatitis C virus, classic swine fever virus, rous sarcoma virus, human immunodeficiency virus, cricket paralysis virus, Kaposi's sarcoma-associated herpesvirus, etc.), a cellular mRNA-derived IRES (e.g. an IRES derived from growth factor mRNAs, such as fibroblast growth factor 2, platelet-derived growth factor B, and vascular endothelial growth factor; an IRES derived from transcription factor mRNAs, such as antennapedia, ultrabithorax, and NF-κB repressing factor; an IRES derived from oncogene mRNAs, such as c-myc, pim-1, and protein kinase p58^{PITSLRE}, etc.), a synthetic IRES (e.g., a CP148 IRES), and others (see e.g., Mokrejs et al. (2007) A Bioinformatical Approach to the Analysis of Viral and Cellular Internal Ribosome Entry Sites. Columbus F editors. New Messenger RNA Research Communications. Hauppauge, N.Y.: Nova Science Publishers; pp. 133-166; see also Mokrejs et al. (2006) Nucleic Acids Res 1; 34(Database issue): D125-30).

In some embodiments, the IRES is a CP148 IRES. An exemplary nucleic acid sequence encoding a CP148 IRES is provided as SEQ ID NO: 584. In some embodiments, the IRES is an EMCV IRES. An exemplary nucleic acid sequence encoding an EMCV IRES is provided as SEQ ID NO: 585.

In some embodiments, the nucleic acid sequence encoding the IRES has at least 80%, at least 85%, at least 90%, at least 91%, at least 92%, at least 93%, at least 94%, at least 95%, at least 96%, at least 97%, at least 98%, at least 99%, or 100% sequence identity to a nucleic acid sequence selected from SEQ ID NO: 584 or SEQ ID NO: 585. In some embodiments, the nucleic acid sequence encoding the IRES has the sequence of SEQ ID NO: 584 or SEQ ID NO: 585.

Exemplary polynucleotides encoding polycistronic mRNAs comprising 1) a first ORF encoding a leader sequence and an antibody light chain (Kappa), 2) an IRES, and 3) second ORF encoding a leader sequence and an antibody heavy chain (IgG1) are provided as SEQ ID NOS: 586-588.

Polynucleotides Encoding Chimeric Polypeptides

In some embodiments, a recombinant nucleic acid (e.g., recombinant herpes virus genome) of the present disclosure comprises one or more polynucleotides encoding a chimeric polypeptide comprising an antibody heavy chain variable region and an antibody light chain variable region separated by a cleavable linker polypeptide. In some embodiments, the chimeric polypeptide comprises a first amino acid sequence comprising an antibody heavy chain and a second amino acid sequence comprising an antibody light chain separated by a cleavable linker polypeptide. In some embodiments, the chimeric polypeptide comprises, from N-terminus to C-terminus, 1) a first amino acid sequence comprising an antibody light chain variable region (e.g., an antibody light chain), 2) a cleavable linker polypeptide, and 3) a second amino acid sequence comprising an antibody heavy chain variable region (e.g., an antibody heavy chain). In some embodiments, the chimeric polypeptide comprises, from N-terminus to C-terminus, 1) a first amino acid sequence comprising an antibody heavy chain variable region (e.g., an antibody heavy chain), 2) a cleavable linker polypeptide, and 3) a second amino acid sequence comprising an antibody light chain variable region (e.g., an antibody light chain).

Any cleavable linker polypeptide known in the art may be used in the chimeric polypeptides of the present disclosure, including, for example, a T2A linker (RAKRGSGEGRGSLLTCGDVEENPGP, SEQ ID NO: 589), a P2A linker (GSGATNFSLLKQAGDVEENPGP, SEQ ID NO: 590), a E2A linker (GSGQCTNYALLKLAGDVESNPGP, SEQ ID NO: 591), an F2A linker (GSGVKQTLNFDLLKLAGDVESNPGP, SEQ ID NO: 592), etc.

In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 589-592. In some embodiments, the linker polypeptide comprises a sequence selected from SEQ ID NOS: 589-592.

Exemplary polynucleotides encoding chimeric polypeptides comprising a leader sequence, an antibody light chain, a linker polypeptide, a leader sequence, and an antibody heavy chain are provided as SEQ ID NOS: 593-595.

Two or More Polynucleotides

In some embodiments, a recombinant nucleic acid (e.g., recombinant herpes virus genome) of the present disclosure comprises two or more polynucleotides encoding an antibody. In some embodiments, the recombinant nucleic acid comprises a first polynucleotide encoding a polypeptide comprising an antibody heavy chain variable region (e.g., a full-length antibody heavy chain), and a second polynucleotide encoding a polypeptide comprising an antibody light chain variable region (e.g., a full-length antibody light chain). In some embodiments, the recombinant nucleic acid comprises two copies of a first polynucleotide encoding a polypeptide comprising an antibody heavy chain variable region (e.g., a full-length antibody heavy chain), and two copies of a second polynucleotide encoding a polypeptide comprising an antibody light chain variable region (e.g., a full-length antibody light chain). In some embodiments, the recombinant nucleic acid comprises two copies of a first polynucleotide encoding a polypeptide comprising an antibody heavy chain variable region (e.g., a full-length antibody heavy chain), and a single copy of a second polynucleotide encoding a polycistronic mRNA comprising a first and second ORF each encoding a polypeptide comprising an antibody light chain variable region (e.g., a full-length antibody light chain) separated by an IRES (e.g., any of the IRESs described herein). In some embodiments, the recombinant nucleic acid comprises a single copy of a first polynucleotide encoding a polycistronic mRNA comprising a first and second ORF each encoding a polypeptide comprising an antibody heavy chain variable region (e.g., a full-length antibody heavy chain) separated by an IRES (e.g., any of the IRESs described herein), and two copies of a second polynucleotide encoding a polypeptide comprising an antibody light chain variable region (e.g., a full-length antibody light chain).

In some embodiments, the recombinant nucleic acid comprises a first polynucleotide encoding a first antibody and a second polynucleotide encoding a second antibody. In some embodiments, the first and second antibody are the same. In some embodiments, the first and second antibodies are different.

Recombinant Nucleic Acids

In some embodiments, the present disclosure relates to recombinant nucleic acids comprising any one or more of the polynucleotides described herein. In some embodiments, the recombinant nucleic acid is a vector (e.g., an expression vector, a display vector, etc.). In some embodiments, the vector is a DNA vector or an RNA vector. Generally, vectors suitable to maintain, propagate, and/or express polynucleotides to produce one or more polypeptides in a subject may be used. Examples of suitable vectors may include, for example, plasmids, cosmids, episomes, transposons, and viral vectors (e.g., adenoviral vectors, adeno-associated viral vectors, vaccinia viral vectors, Sindbis-viral vectors, measles vectors, herpes viral vectors, lentiviral vectors, retroviral vectors, etc.). In some embodiments, the vector is a herpes viral vector. In some embodiments, the vector is capable of autonomous replication in a host cell. In some embodiments, the vector is incapable of autonomous replication in a host cell. In some embodiments, the vector can integrate into a host DNA. In some embodiments, the vector cannot integrate into a host DNA (e.g., is episomal). Methods of making vectors containing one or more polynucleotides of interest are well known to one of ordinary skill in the art, including, for example, by chemical synthesis, or by artificial manipulation of isolated segments of nucleic acids (e.g., by genetic engineering techniques).

In some embodiments, a recombinant nucleic acid of the present disclosure is a herpes simplex virus (HSV) amplicon. Herpes virus amplicons, including the structural features and methods of making the same, are generally known to one of ordinary skill in the art (see e.g., de Silva S. and Bowers W. “Herpes Virus Amplicon Vectors”. Viruses 2009, 1, 594-629). In some embodiments, the herpes simplex virus amplicon is an HSV-1 amplicon. In some embodiments, the herpes simplex virus amplicon is an HSV-1 hybrid amplicon. Examples of HSV-1 hybrid amplicons may include, but are not limited to, HSV/AAV hybrid amplicons, HSV/EBV hybrid amplicons, HSV/EBV/RV hybrid amplicons, and/or HSV/Sleeping Beauty hybrid amplicons. In some embodiments, the amplicon is an HSV/AAV hybrid amplicon. In some embodiments, the amplicon is an HSV/Sleeping Beauty hybrid amplicon.

In some embodiments, a recombinant nucleic acid of the present disclosure is a recombinant herpes virus genome. The recombinant herpes virus genome may be a recombinant genome from any member of the Herpesviridae family of DNA viruses known in the art, including, for example, a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any combinations thereof or any derivatives thereof. In some embodiments, the recombinant herpes virus genome comprises more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes virus genes. In some embodiments, the recombinant herpes virus genome is attenuated (e.g., as compared to a corresponding, wild-type herpes virus genome). In some embodiments, the recombinant herpes virus genome is replication competent. In some embodiments, the recombinant herpes virus genome is replication defective.

In some embodiments, the recombinant nucleic acid is a recombinant herpes simplex virus (HSV) genome. In some embodiments, the recombinant herpes simplex virus genome is a recombinant type 1 herpes simplex virus (HSV-1) genome, a recombinant type 2 herpes simplex virus (HSV-2) genome, or any derivatives thereof. In some embodiments, the recombinant herpes simplex virus genome is a recombinant HSV-1 genome. In some embodiments, the recombinant herpes simplex virus genome is replication competent. In some embodiments, the recombinant herpes simplex virus genome is replication defective. In some embodiments, the recombinant herpes simplex virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes simplex virus genes. As used herein, an “inactivating mutation” may refer to any mutation that results in the gene product (RNA or protein) having reduced, undetectable, or eliminated quantity and/or function (e.g., as compared to a corresponding sequence lacking the inactivating mutation). Examples of inactivating mutations may include, but are not limited to, deletions, insertions, point mutations, and rearrangements in transcriptional control sequences (promoters, enhancers, insulators, etc.) and/or coding sequences of a given gene or regulon. Any suitable method of measuring the quantity of a gene or regulon product known in the art may be used, including, for example, qPCR, Northern blots, RNAseq, western blots, ELISAs, etc.

In some embodiments, the recombinant herpes simplex virus genome comprises an inactivating mutation in at least one, at least two, at least three, at least four, at least five, at least six, at least seven, or all eight of the Infected Cell Protein (or Infected Cell Polypeptide) (ICP) 0, ICP4, ICP22, ICP27, ICP47, thymidine kinase (tk), Long Unique Region (UL) 41 and/or UL55 herpes simplex virus genes. In some embodiments, the recombinant herpes simplex virus genome does not comprise an inactivating mutation in the ICP34.5 (one or both copies) and/or ICP47 herpes simplex virus genes (e.g., to avoid production of an immune-stimulating virus). In some embodiments, the recombinant herpes simplex virus genome does not comprise an inactivating mutation in the ICP34.5 (one or both copies) herpes simplex virus gene. In some embodiments, the recombinant herpes simplex virus genome does not comprise an inactivating mutation in the ICP47 herpes simplex virus gene. In some embodiments, the recombinant herpes simplex virus genome does not comprise an inactivating mutation in the ICP34.5 (one or both copies) and ICP47 herpes simplex virus genes. In some embodiments, the recombinant herpes simplex virus genome is not oncolytic.

In some embodiments, the recombinant herpes simplex virus genome comprises an inactivating mutation in the ICP0 gene (one or both copies). In some embodiments, the recombinant herpes simplex virus genome comprises an inactivating mutation in the ICP0 gene (one or both copies), and further comprises an initiating mutation in the ICP4 (one or both copies) ICP22, ICP27, ICP47, UL41, and/or UL55 genes. In some embodiments, the recombinant herpes simplex virus genome comprises an inactivating mutation in the ICP0 gene (one or both copies), and an inactivating mutation in the ICP4 gene (one or both copies). In some embodiments, the recombinant herpes simplex virus genome comprises an inactivating mutation in the ICP0 gene (one or both copies), and an inactivating mutation in the ICP22 gene. In some embodiments, the recombinant herpes simplex virus genome comprises an inactivating mutation in the ICP0 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), and an inactivating mutation in the ICP22 gene. In some embodiments, the recombinant herpes simplex virus genome comprises an inactivating mutation in the ICP0 gene (one or both copies), an inactivating mutation in the ICP4 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP22 gene, and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP0 (one or both copies), ICP4 (one or both copies), ICP22, and/or UL41 genes. In some embodiments, the recombinant herpes simplex virus genome further comprises an inactivating mutation in the ICP27, ICP47, and/or UL55 genes.

In some embodiments, the recombinant herpes simplex virus genome comprises an inactivating mutation in the ICP4 gene (one or both copies). In some embodiments, the recombinant herpes complex virus genome comprises an inactivating mutation in the ICP4 (one or both copies, and further comprises an inactivating mutation in the ICP0 (one or both copies), ICP22, ICP27, ICP47, UL41, and/or UL55 genes. In some embodiments, the recombinant herpes simplex virus genome comprises an inactivating mutation in the ICP4 gene (one or both copies), and an inactivating mutation in the ICP22 gene. In some embodiments, the recombinant herpes simplex virus genome comprises an inactivating mutation in the ICP4 gene (one or both copies), and an inactivating mutation in the UL41 gene. In some embodiments, the recombinant herpes simplex virus genome comprises an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP4 (one or both copies), ICP22, and/or UL41 genes. In some embodiments, the recombinant herpes simplex virus genome further comprises an inactivating mutation in the ICP0, ICP27, ICP47, and/or UL55 genes.

In some embodiments, the recombinant herpes simplex virus genome comprises an inactivating mutation in the ICP22 gene. In some embodiments, the recombinant herpes simplex virus genome comprises an inactivating mutation in the ICP22 gene, and further comprises an inactivating mutation in the ICP0 (one or both copies), ICP4 (one or both copies), ICP27, ICP47, UL41, and/or UL55 genes. In some embodiments, the recombinant herpes simplex virus genome comprises an inactivating mutation in the ICP22 gene, and an inactivating mutation UL41 gene. In some embodiments, the inactivating mutation is a deletion of the coding sequence of the ICP22 and/or UL41 genes. In some embodiments, the recombinant herpes simplex virus genome further comprises an inactivating mutation in the ICP0 (one or both copies), ICP4 (one or both copies), ICP27, ICP47, and/or UL55 genes.

In some embodiments, the recombinant herpes simplex virus genome comprises an inactivating mutation in the ICP27 gene. In some embodiments, the recombinant herpes simplex virus genome comprises an inactivating mutation in the ICP27 gene, and further comprises an inactivating mutation in the ICP0 (one or both copies), ICP4 (one or both copies), ICP22, ICP47, UL41, and/or UL55 genes. In some embodiments, the inactivating mutation is a deletion of the coding sequence of the ICP27 gene.

In some embodiments, the recombinant herpes simplex virus genome comprises an inactivating mutation in the ICP47 gene. In some embodiments, the recombinant herpes simplex virus genome comprises an inactivating mutation in the ICP47 gene, and further comprises an inactivating mutation in the ICP0 (one or both copies), ICP4 (one or both copies), ICP22, ICP27, UL41, and/or UL55 genes. In some embodiments, the inactivating mutation is a deletion of the coding sequence of the ICP47 gene.

In some embodiments, the recombinant herpes simplex virus genome comprises an inactivating mutation in the UL41 gene. In some embodiments, the recombinant herpes simplex virus genome comprises an inactivating mutation in the UL41 gene, and further comprises an inactivating mutation in the ICP0 (one or both copies), ICP4 (one or both copies), ICP22, ICP27, ICP47, and/or UL55 genes. In some embodiments, the inactivating mutation is a deletion of the coding sequence of the UL41 gene.

In some embodiments, the recombinant herpes simplex virus genome comprises an inactivating mutation in the UL55 gene. In some embodiments, the recombinant herpes simplex virus genome comprises an inactivating mutation in the UL55 gene, and further comprises an inactivating mutation in the ICP0 (one or both copies), ICP4 (one or both copies), ICP22, ICP27, ICP47, and/or UL41 genes. In some embodiments, the inactivating mutation is a deletion of the coding sequence of the UL55 gene.

In some embodiments, the recombinant herpes simplex virus genome comprises an inactivating mutation in (e.g., a deletion of) the internal repeat (Joint) region comprising the internal repeat long (IRL) and internal repeat short (IRS) regions. In some embodiments, inactivation (e.g., deletion) of the Joint region eliminates one copy each of the ICP4 and ICP0 genes. In some embodiments, inactivation (e.g., deletion) of the Joint region further inactivates (e.g., deletes) the promoter for the ICP22 and ICP47 genes. If desired, expression of one or both of these genes can be restored by insertion of an immediate early promoter into the recombinant herpes simplex virus genome (see e.g., Hill et al. (1995). Nature 375(6530): 411-415; Goldsmith et al. (1998). J Exp Med 187(3): 341-348). Without wishing to be bound by theory, it is believed that inactivating (e.g., deleting) the Joint region may contribute to the stability of the recombinant herpes simplex virus genome and/or allow for the recombinant herpes simplex virus genome to accommodate more and/or larger transgenes.

In some embodiments, the recombinant herpes simplex virus genome comprises an inactivating mutation in the ICP4 (one or both copies), ICP22, and ICP27 genes. In some embodiments, the recombinant herpes simplex virus genome comprises an inactivating mutation in the ICP4 (one or both copies), ICP27, and UL55 genes. In some embodiments, the recombinant herpes simplex virus genome comprises an inactivating mutation in the ICP4 (one or both copies), ICP22, ICP27, ICP47, and UL55 genes. In some embodiments, the inactivating mutation in the ICP4 (one or both copies), ICP27, and/or UL55 genes is a deletion of the coding sequence of the ICP4 (one or both copies), ICP27, and/or UL55 genes. In some embodiments, the inactivating mutation in the ICP22 and ICP47 genes is a deletion in the promoter region of the ICP22 and ICP47 genes (e.g., the ICP22 and ICP47 coding sequences are intact but are not transcriptionally active). In some embodiments, the recombinant herpes simplex virus genome comprises a deletion in the coding sequence of the ICP4 (one or both copies), ICP27, and UL55 genes, and a deletion in the promoter region of the ICP22 and ICP47 genes. In some embodiments, the recombinant herpes simplex virus genome further comprises an inactivating mutation in the ICP0 (one or both copies) and/or UL41 genes.

In some embodiments, the recombinant herpes simplex virus genome comprises an inactivating mutation in the ICP0 (one or both copies) gene. In some embodiments, the recombinant herpes simplex virus genome comprises an inactivating mutation in the ICP0 (one or both copies) and ICP4 (one or both copies) genes. In some embodiments, the recombinant herpes simplex virus genome comprises an inactivating mutation in the ICP0 (one or both copies), ICP4 (one or both copies), and ICP22 genes. In some embodiments, the recombinant herpes simplex virus genome comprises an inactivating mutation in the ICP0 (one or both copies), ICP4 (one or both copies), ICP22, and ICP27 genes. In some embodiments, the recombinant herpes simplex virus genome comprises an inactivating mutation in the ICP0 (one or both copies), ICP4 (one or both copies), ICP22, ICP27 and UL55 genes. In some embodiments, the inactivating mutation in the ICP0 (one or both copies), ICP4 (one or both copies), ICP22, ICP27 and/or UL55 genes comprises a deletion of the coding sequence of the ICP0 (one or both copies), ICP4 (one or both copies), ICP22, ICP27 and/or UL55 genes. In some embodiments, the recombinant herpes simplex virus genome further comprises an inactivating mutation in the ICP47 and/or the UL41 genes.

In some embodiments, a recombinant herpes simplex virus genome comprises one or more polynucleotides of the present disclosure within one, two, three, four, five, six, seven or more viral gene loci. Examples of suitable viral loci may include, without limitation, the ICP0 (one or both copies), ICP4 (one or both copies), ICP22, ICP27, ICP47, tk, UL41 and UL55 herpes simplex viral gene loci. In some embodiments, a recombinant herpes simplex virus genome comprises one or more polynucleotides of the present disclosure within one or both of the viral ICP4 gene loci (e.g., a recombinant virus carrying a polynucleotide encoding an antibody (or a portion thereof) in one or both of the ICP4 loci). In some embodiments, a recombinant herpes simplex virus genome comprises one or more polynucleotides of the present disclosure within the viral ICP22 gene locus (e.g., a recombinant virus carrying a polynucleotide encoding an antibody (or a portion thereof) in the ICP22 locus). In some embodiments, a recombinant herpes simplex virus genome comprises one or more polynucleotides of the present disclosure within the viral UL41 gene locus (e.g., a recombinant virus carrying a polynucleotide encoding an antibody (or a portion thereof) in the UL41 locus). In some embodiments, a recombinant herpes simplex virus genome comprises one or more polynucleotides of the present disclosure within the viral ICP47 gene locus (e.g., a recombinant virus carrying a polynucleotide encoding an antibody (or a portion thereof) in the ICP47 locus). In some embodiments, a recombinant herpes simplex virus genome comprises one or more polynucleotides of the present disclosure within one or both of the viral ICP4 gene loci, and one or more polynucleotides of the present disclosure within the viral ICP22 gene locus (e.g., a recombinant virus carrying a polynucleotide encoding an antibody heavy chain in one or both of the ICP4 loci, and a polynucleotide encoding an antibody light chain in the ICP22 locus; a recombinant virus carrying a polynucleotide encoding an antibody heavy chain in one or both of the ICP4 loci, and a polynucleotide encoding a polycistronic mRNA encoding two copies of an antibody light chain in the ICP22 locus; etc.). In some embodiments, a recombinant herpes simplex virus genome comprises one or more polynucleotides of the present disclosure within one or both of the viral ICP4 gene loci, and one or more polynucleotides of the present disclosure within the viral UL41 gene locus (e.g., a recombinant virus carrying a polynucleotide encoding an antibody heavy chain in one or both of the ICP4 loci, and a polynucleotide encoding an antibody light chain in the UL41 locus; a recombinant virus carrying a polynucleotide encoding an antibody heavy chain in one or both of the ICP4 loci, and a polynucleotide encoding a polycistronic mRNA encoding two copies of an antibody light chain in the UL41 locus; etc.). In some embodiments, a recombinant herpes simplex virus genome comprises one or more polynucleotides of the present disclosure within the viral UL41 gene locus, and one or more polynucleotides of the present disclosure within the viral ICP22 gene locus (e.g., a recombinant virus carrying a polynucleotide encoding an antibody heavy chain in the UL41 locus, and a polynucleotide encoding an antibody light chain in the ICP22 locus; a recombinant virus carrying a polynucleotide encoding an antibody light chain in the UL41 locus, and a polynucleotide encoding an antibody heavy chain in the ICP22 locus; etc.). In some embodiments, a recombinant herpes simplex virus genome comprises one or more polynucleotides of the present disclosure within one or more of the viral ICP4 gene loci, one or more polynucleotides of the present disclosure within the viral ICP22 gene locus, and one or more polynucleotides of the present disclosure within the viral UL41 gene locus (e.g., a recombinant virus carrying a polynucleotide encoding an antibody heavy chain in one or both of the ICP4 loci, a polynucleotide encoding an antibody light chain in the ICP22 locus, and a polynucleotide encoding an antibody light chain in the UL41 locus; a recombinant virus carrying a polynucleotide encoding an antibody light chain in one or both of the ICP4 loci, a polynucleotide encoding an antibody heavy chain in the ICP22 locus, and a polynucleotide encoding an antibody heavy chain in the UL41 locus; etc.)

In some embodiments, the recombinant herpes virus genome (e.g., a recombinant herpes simplex virus genome) has been engineered to decrease or eliminate expression of one or more toxic herpes virus genes (such as one or both copies of the HSV ICP4 gene, the ICP22 gene, the UL41 gene, and/or the ICP27 gene). In some embodiments, the recombinant herpes virus genome (e.g., recombinant herpes simplex virus genome) has been engineered to reduce cytotoxicity of the recombinant genome (e.g., when introduced into a target cell) as compared to a corresponding wild-type herpes virus genome (e.g., a wild-type herpes simplex virus genome). In some embodiments, cytotoxicity (e.g., in human keratinocytes and/or fibroblast cells) of the recombinant virus genome (e.g., a recombinant herpes simplex virus genome) is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% as compared to a corresponding wild-type herpes virus genome (e.g., measuring the relative cytotoxicity of a recombinant ΔICP4 (one or both copies) herpes simplex virus genome vs. a wild-type herpes simplex virus genome in human keratinocytes or fibroblasts (primary cells or cell lines); measuring the relative cytotoxicity of a recombinant ΔICP4 (one or both copies)/ΔICP22 herpes simplex virus genome vs. a wild-type herpes simplex virus genome in human keratinocytes or fibroblasts (primary cells or cell lines); etc.). In some embodiments, cytotoxicity (e.g., in human keratinocytes and/or fibroblast cells) of the recombinant herpes virus genome (e.g., a recombinant herpes simplex virus genome) is reduced by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more as compared to a corresponding wild-type herpes virus genome (e.g., measuring the relative cytotoxicity of a recombinant ΔICP4 (one or both copies) herpes simplex virus genome vs. a wild-type herpes simplex virus genome in human keratinocytes or fibroblasts (primary cells or cell lines); measuring the relative cytotoxicity of a recombinant ΔICP4 (one or both copies)/ΔICP22 herpes simplex virus genome vs. a wild-type herpes simplex virus genome in human keratinocytes or fibroblasts (primary cells or cell lines); etc.). Methods of measuring cytotoxicity are known to one of ordinary skill in the art, including, for example, through the use of vital dyes (formazan dyes), protease biomarkers, an MTT assay (or an assay using related tetrazolium salts such as XTT, MTS, water-soluble tetrazolium salts, etc.), measuring ATP content, etc.

In some embodiments, the recombinant herpes virus genome (e.g., a recombinant herpes simplex virus genome) has been engineered to reduce its impact on host cell proliferation after exposure of a target cell to the recombinant genome, as compared to a corresponding wild-type herpes virus genome (e.g., a wild-type herpes simplex virus genome). In some embodiments, the target cell is a human cell. In some embodiments, the target cell is a cell of the epidermis and/or dermis. In some embodiment, the target cell is a cell of the eye. In some embodiments, the target cell is a cell of the joint. In some embodiments, the target cell is a cell of the lungs. In some embodiments, host cell proliferation (e.g., of human keratinocytes and/or fibroblast cells) after exposure to the recombinant genome is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% faster as compared to host cell proliferation after exposure to a corresponding wild-type herpes virus genome (e.g., measuring the relative cellular proliferation after exposure to a recombinant ΔICP4 (one or both copies) herpes simplex virus genome vs. cellular proliferation after exposure to a wild-type herpes simplex virus genome in human keratinocytes or fibroblasts (primary cells or cell lines); measuring the relative cellular proliferation after exposure to a recombinant ΔICP4 (one or both copies)/ΔICP22 herpes simplex virus genome vs. cellular proliferation after exposure to a wild-type herpes simplex virus genome in human keratinocytes or fibroblasts (primary cells or cell lines); etc.). In some embodiments, host cell proliferation (e.g., of human keratinocytes and/or fibroblast cells) after exposure to the recombinant genome is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, or at least about 1000-fold faster as compared to host cell proliferation after exposure to a corresponding wild-type herpes virus genome (e.g., measuring the relative cellular proliferation after exposure to a recombinant ΔICP4 (one or both copies) herpes simplex virus genome vs. cellular proliferation after exposure to a wild-type herpes simplex virus genome in human keratinocytes or fibroblasts (primary cells or cell lines); measuring the relative cellular proliferation after exposure to a recombinant ΔICP4 (one or both copies)/ΔICP22 herpes simplex virus genome vs. cellular proliferation after exposure to a wild-type herpes simplex virus genome in human keratinocytes or fibroblasts (primary cells or cell lines); etc.). Methods of measuring cellular proliferation are known to one of ordinary skill in the art, including, for example, through the use of a Ki67 cell proliferation assay, a BrdU cell proliferation assay, etc.

A vector (e.g., herpes viral vector) may include one or more polynucleotides of the present disclosure in a form suitable for expression of the polynucleotide in a host cell. Vectors may include one or more regulatory sequences operatively linked to the polynucleotide to be expressed (e.g., as described above).

In some embodiments, a recombinant nucleic acid (e.g., a recombinant herpes simplex virus genome) of the present disclosure comprises one or more of the polynucleotides described herein inserted in any orientation in the recombinant nucleic acid. If the recombinant nucleic acid comprises two or more polynucleotides described herein (e.g., two or more, three or more, etc.), the polynucleotides may be inserted in the same orientation or opposite orientations to one another. Without wishing to be bound be theory, incorporating two polynucleotides (e.g., two transgenes) into a recombinant nucleic acid (e.g., a vector) in an antisense orientation may help to avoid read-through and ensure proper expression of each polynucleotide.

### IV. Viruses

Certain aspects of the present disclosure relate to viruses comprising any of the polynucleotides and/or recombinant nucleic acids described herein. In some embodiments, the virus is capable of infecting one or more target cells of a subject (e.g., a human) In some embodiments, the virus is suitable for delivering the polynucleotides and/or recombinant nucleic acid into one or more target cells of a subject (e.g., a human subject). In some embodiments, the one or more target cells are one or more cells of the mucosa or skin (e.g., one or more cells of the epidermis, dermis, and/or subcutis). In some embodiments, the one or more cells are selected from keratinocytes, melanocytes, Langerhans cells, Merkel cells, mast cells, fibroblasts, and/or adipocytes. In some embodiments, the one or more cells are keratinocytes. In some embodiments, the one or more cells reside in the stratum corneum, stratum granulosum, stratum spinulosum, stratum basale, and/or basement membrane. In some embodiments, the one or more target cells are one or more epidermal cells. In some embodiments, the one or more target cells are one or more dermal cells. In some embodiments, the one or more target cells are one or more cells of a joint. In some embodiments, the one or more target cells are one or more cells of the eye. In some embodiments, the one or more target cells are one or more cells of the airway and/or lung.

Any suitable virus known in the art may be used, including, for example, adenovirus, adeno-associated virus, retrovirus, lentivirus, sendai virus, herpes virus (e.g., a herpes simplex virus), vaccinia virus, and/or any hybrid or derivative viruses thereof. In some embodiments, the virus is attenuated. In some embodiments, the virus is replication defective. In some embodiments, the virus is replication competent. In some embodiments, the virus has been modified to alter its tissue tropism relative to the tissue tropism of a corresponding unmodified, wild-type virus. In some embodiments, the virus has reduced cytotoxicity as compared to a corresponding wild-type virus. Methods of producing a virus comprising recombinant nucleic acids are well known to one of ordinary skill in the art.

In some embodiments, the virus is a member of the Herpesviridae family of DNA viruses, including, for example, a herpes simplex virus, a varicella zoster virus, a human cytomegalovirus, a herpesvirus 6A, a herpesvirus 6B, a herpesvirus 7, and a Kaposi's sarcoma-associated herpesvirus, etc. In some embodiments, the herpes virus is attenuated. In some embodiments, the herpes virus is replication defective. In some embodiments, the herpes virus is replication competent. In some embodiments, the herpes virus has reduced cytotoxicity as compared to a corresponding wild-type herpes virus. In some embodiments, the herpes virus is not oncolytic.

In some embodiments, the virus is a herpes simplex virus. Herpes simplex viruses comprising recombinant nucleic acids may be produced by a process disclosed, for example, in WO2015/009952 and/or WO2017/176336. In some embodiments, the herpes simplex virus is attenuated. In some embodiments, the herpes simplex virus is replication defective. In some embodiments, the herpes simplex virus is replication competent. In some embodiments, the herpes simplex virus is a herpes simplex type 1 virus (HSV-1), a herpes simplex type 2 virus (HSV-2), or any derivatives thereof. In some embodiments, the herpes simplex virus is a herpes simplex type 1 virus (HSV-1). In some embodiments, the HSV-1 is attenuated. In some embodiments, the HSV-1 is replication defective. In some embodiments, the HSV-1 is replication competent. In some embodiments, the HSV-1 has reduced cytotoxicity as compared to a corresponding wild-type HSV-1. In some embodiments, the HSV-1 is not oncolytic.

In some embodiments, the herpes simplex virus has been modified to alter its tissue tropism relative to the tissue tropism of an unmodified, wild-type herpes simplex virus. In some embodiments, the herpes simplex virus comprises a modified envelope. In some embodiments, the modified envelope comprises one or more (e.g., one or more, two or more, three or more, four or more, etc.) mutant herpes simplex virus glycoproteins. Examples of herpes simplex virus glycoproteins may include, but are not limited to, the glycoproteins gB, gC, gD, gH, and gL. In some embodiments, the modified envelope alters the herpes simplex virus tissue tropism relative to a wild-type herpes simplex virus

In some embodiments, the transduction efficiency (in vitro and/or in vivo) of a virus of the present disclosure (e.g., a herpes virus such as a herpes simplex virus) for one or more target cells (e.g., one or more human keratinocytes and/or fibroblasts) is at least about 25%. For example, the transduction efficiency of the virus for one or more target cells may be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 99.5%, or more. In some embodiments, the virus is a herpes simplex virus and the transduction efficiency of the virus for one or more target cells (e.g., one or more human keratinocytes and/or fibroblasts) is about 85% to about 100%. In some embodiments, the virus is a herpes simplex virus and the transduction efficiency of the virus for one or more target cells (e.g., one or more human keratinocytes and/or fibroblasts) is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100%. Methods of measuring viral transduction efficiency in vitro or in vivo are well known to one of ordinary skill in the art, including, for example, qPCR analysis, deep sequencing, western blotting, fluorometric analysis (such as fluorescent in situ hybridization (FISH), fluorescent reporter gene expression, immunofluorescence, FACS), etc.

### V. Pharmaceutical Compositions and Formulations

Certain aspects of the present disclosure relate to pharmaceutical compositions and formulations comprising any of the recombinant nucleic acids (e.g., a recombinant herpes virus genome) and/or viruses (e.g., a herpes virus comprising a recombinant genome) described herein, and a pharmaceutically acceptable excipient or carrier.

In some embodiments, the pharmaceutical composition or formulation comprises any one or more of the viruses (e.g., herpes viruses) as described herein. In some embodiments, the pharmaceutical composition or formulation comprises from about 10⁴ to about 10¹² plaque forming units (PFU)/mL of the virus. For example, the pharmaceutical composition or formulation may comprise from about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10⁴ to about 10⁹, about 10⁵ to about 10⁹, about 10⁶ to about 10⁹, about 10⁷ to about 10⁹, about 10⁸ to about 10⁹, about 10⁴ to about 10⁸, about 10⁵ to about 10⁸, about 10⁶ to about 108, about 10⁷ to about 10⁸, about 10⁴ to about 10⁷, about 10⁵ to about 10⁷, about 10⁶ to about 10⁷, about 10⁴ to about 10⁶, about 10⁵ to about 10⁶, or about 10⁴ to about 10⁵ PFU/mL of the virus. In some embodiments, the pharmaceutical composition or formulation comprises about 10⁴, about 10⁵, about 10⁶, about 10⁷, about 10⁸, about 10⁹, about 10¹⁰, about 10¹¹, or about 10¹² PFU/mL of the virus.

Pharmaceutical compositions and formulations can be prepared by mixing the active ingredient(s) (such as a recombinant nucleic acid and/or a virus) having the desired degree of purity with one or more pharmaceutically acceptable carriers or excipients. Pharmaceutically acceptable carriers or excipients are generally nontoxic to recipients at the dosages and concentrations employed, and may include, but are not limited to: buffers (such as phosphate, citrate, acetate, and other organic acids); antioxidants (such as ascorbic acid and methionine); preservatives (such as octadecyldimethylbenzyl ammonium chloride, benzalkonium chloride, benzethonium chloride, phenol, butyl or benzyl alcohol, alkyl parabens, catechol, resorcinol, cyclohexanol, 3-pentanol, and m-cresol); amino acids (such as glycine, glutamine, asparagine, histidine, arginine, or lysine); low molecular weight (less than about 10 residues) polypeptides; proteins (such as serum albumin, gelatin, or immunoglobulins); polyols (such as glycerol, e.g., formulations including 10% glycerol); hydrophilic polymers (such as polyvinylpyrrolidone); monosaccharides, disaccharides, and other carbohydrates (including glucose, mannose, or dextrins); chelating agents (such as EDTA); sugars (such as sucrose, mannitol, trehalose, or sorbitol); salt-forming counter-ions (such as sodium); metal complexes (such as Zn-protein complexes); and/or non-ionic surfactants (such as polyethylene glycol (PEG)). A thorough discussion of pharmaceutically acceptable carriers is available in REMINGTON'S PHARMACEUTICAL SCIENCES (Mack Pub. Co., N.J. 1991).

In some embodiments, the pharmaceutical composition or formulation comprises one or more lipid (e.g., cationic lipid) carriers. In some embodiments, the pharmaceutical composition or formulation comprises one or more nanoparticle carriers. Nanoparticles are submicron (less than about 1000 nm) sized drug delivery vehicles that can carry encapsulated drugs (such as synthetic small molecules, proteins, peptides, cells, viruses, and nucleic acid-based biotherapeutics for rapid or controlled release. A variety of molecules (e.g., proteins, peptides, recombinant nucleic acids, etc.) can be efficiently encapsulated in nanoparticles using processes well known in the art. In some embodiments, a molecule “encapsulated” in a nanoparticle may refer to a molecule (such as a virus) that is contained within the nanoparticle or attached to and/or associated with the surface of the nanoparticle, or any combination thereof. Nanoparticles for use in the compositions or formulations described herein may be any type of biocompatible nanoparticle known in the art, including, for example, nanoparticles comprising poly(lactic acid), poly(glycolic acid), PLGA, PLA, PGA, and any combinations thereof (see e.g., Vauthier et al. Adv Drug Del Rev. (2003) 55: 519-48; US2007/0148074; US2007/0092575; US2006/0246139; U.S. Pat. Nos. 5,753,234; 7,081,483; and WO2006/052285).

In some embodiments, the pharmaceutically acceptable carrier or excipient may be adapted for or suitable for any administration route known in the art, including, for example, intravenous, intramuscular, subcutaneous, cutaneous, oral, intratracheal, sublingual, buccal, topical, transdermal, intradermal, intraperitoneal, intraorbital, intravitreal, subretinal, transmucosal, intraarticular, by implantation, by inhalation, intrathecal, intraventricular, and/or intranasal administration. In some embodiments, the pharmaceutically acceptable carrier or excipient is adapted for or suitable for topical, transdermal, subcutaneous, and/or intradermal administration. In some embodiments, the pharmaceutical composition or formulation is adapted for or suitable for topical, transdermal, subcutaneous, and/or intradermal administration. In some embodiments, the pharmaceutically acceptable carrier or excipient is adapted for or suitable for topical, transdermal, and/or intradermal administration. In some embodiments, the pharmaceutical composition or formulation is adapted for or suitable for topical, transdermal, and/or intradermal administration. In some embodiments, the pharmaceutically acceptable carrier or excipient is adapted for or suitable for oral, sublingual, nasal, intranasal, intratracheal, or buccal administration, or administration via inhalation. In some embodiments, the pharmaceutical composition or formulation is adapted for or suitable for oral, sublingual, nasal, intranasal, intratracheal, or buccal administration, or administration via inhalation. In some embodiments, the pharmaceutically acceptable carrier or excipient is adapted for or suitable for topical (to the eye), intravitreal, subretinal, or intraorbital administration. In some embodiments, the pharmaceutical composition or formulation is adapted for or suitable for topical (to the eye), intravitreal, subretinal, or intraorbital administration. In some embodiments, the pharmaceutically acceptable carrier or excipient is adapted for or suitable for intraarticular administration. In some embodiments, the pharmaceutical composition or formulation is adapted for or suitable for intraarticular administration.

Examples of carriers or excipients adapted for or suitable for use in pharmaceutical compositions or formulations of the present disclosure may include, but are not limited to, ointments, oils, pastes, creams, aerosols, suspensions, emulsions, fatty ointments, gels, powders, liquids, lotions, solutions, sprays, patches (e.g., transdermal patches or microneedle patches), adhesive strips, a microneedle or microneedle arrays, and inhalants. In some embodiments, the carrier or excipient (e.g., the pharmaceutically acceptable carrier or excipient) comprises one or more (e.g., one or more, two or more, three or more, four or more, five or more, etc.) of an ointment, oil, paste, cream, aerosol, suspension, emulsion, fatty ointment, gel, powder, liquid, lotion, solution, spray, adhesive strip, and an inhalant. In some embodiments, the carrier comprises a patch (e.g. a patch that adheres to the skin), such as a transdermal patch or a microneedle patch. In some embodiments, the carrier comprises a microneedle or microneedle array. Methods for making and using microneedle arrays suitable for composition delivery are generally known in the art (Kim Y. et al. “Microneedles for drug and vaccine delivery”. Advanced Drug Delivery Reviews 2012, 64 (14): 1547-68).

In some embodiments, the pharmaceutical composition or formulation further comprises one or more additional components. Examples of additional components may include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; hydroxymethylcellulose; polyvinylpyrrolidone; sweetenings; flavorings; perfuming agents; colorants; moisturizers; sunscreens; antibacterial agents; agents able to stabilize polynucleotides or prevent their degradation, and the like. In some embodiments, the pharmaceutical composition or formulation comprises a hydroxypropyl methylcellulose gel. In some embodiments, the pharmaceutical composition or formulation comprises a phosphate buffer. In some embodiments, the pharmaceutical composition or formulation comprises glycerol (e.g., at about 1%, about 2%, about 3%, about 4%, about 5%, about 6%, about 7%, about 8%, about 9%, about 10%, about 15%, etc.).

Compositions and formulations (e.g., pharmaceutical compositions and formulations) to be used for in vivo administration are generally sterile. Sterility may be readily accomplished, e.g., by filtration through sterile filtration membranes.

In some embodiments, any of the recombinant nucleic acids, viruses, and/or pharmaceutical compositions or formulations described herein may be used to deliver one or more polynucleotides encoding an antibody (e.g., a therapeutic antibody) into one or more cells of a subject and/or may be used to express an antibody (e.g., a therapeutic antibody) in one or more tissues of a subject. In some embodiments, any of the recombinant nucleic acids, viruses, and/or pharmaceutical compositions or formulations described herein may be used in a therapy. In some embodiments, any of the recombinant nucleic acids, viruses, and/or pharmaceutical compositions or formulations may be used in the treatment of a disease or condition that would benefit from the administration of an antibody (e.g., a therapeutic antibody). In some embodiments, any of the recombinant nucleic acids, viruses, and/or pharmaceutical compositions or formulations described herein may be used in the treatment of one or more of psoriasis (e.g., chronic plaque psoriasis), atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer (e.g., skin cancer, breast cancer, lymphoma, colorectal cancer, head and neck cancer, etc.), hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, autoimmune disease (e.g., multiple sclerosis, systemic lupus erythematosus, inflammatory bowel disease, Addison's disease, Graves' disease, Sjogren's syndrome, Hashimoto's thyroiditis, Myasthenia gravis, vasculitis, pernicious anemia, celiac disease, etc.), asthma, uveal melanoma, thyroid eye disease, infectious diseases, graft/tissue/organ rejection, and/or neurological diseases (e.g., Alzheimer's).

In some embodiments, any of the recombinant nucleic acids, viruses, and/or pharmaceutical compositions or formulations described herein may be used in the preparation or manufacture of a medicament. In some embodiments, any of the recombinant nucleic acids, viruses, and/or pharmaceutical compositions or formulations described herein may be used in the preparation or manufacture of a medicament useful for delivering one or more polynucleotides encoding an antibody (e.g., a therapeutic antibody) into one or more cells of a subject and/or may be used in the preparation or manufacture of a medicament useful for expressing an antibody (e.g., a therapeutic antibody) in one or more tissues of a subject. In some embodiments, any of the recombinant nucleic acids, viruses, and/or pharmaceutical compositions or formulations described herein may be used in the preparation or manufacture of a medicament useful for the treatment of a disease or condition that would benefit from the administration of an antibody (e.g., a therapeutic antibody). In some embodiments, any of the recombinant nucleic acids, viruses, and/or pharmaceutical compositions or formulations described herein may be used in the preparation or manufacture of a medicament useful for the treatment of one or more of psoriasis (e.g., chronic plaque psoriasis), atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer (e.g., skin cancer, breast cancer, lymphoma, colorectal cancer, head and neck cancer, etc.), hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, autoimmune disease (e.g., multiple sclerosis, systemic lupus erythematosus, inflammatory bowel disease, Addison's disease, Graves' disease, Sjogren's syndrome, Hashimoto's thyroiditis, Myasthenia gravis, vasculitis, pernicious anemia, celiac disease, etc.), asthma, uveal melanoma, thyroid eye disease, infectious diseases, graft/tissue/organ rejection, and/or neurological diseases (e.g., Alzheimer's)

### VI. Methods

Certain aspects of the present disclosure relate to methods of delivering an antibody to a subject comprising administering to the subject an effective amount of any of the recombinant nucleic acids, viruses, medicaments, and/or compositions or formulations described herein. In some embodiments, the present disclosure relates to a method of locally delivering an antibody to one or more specific tissues of interest in a subject (e.g., tissues of the eye, tissues of the joints, tissues of the skin, tissues of the lungs, etc.) comprising administering to the subject an effective amount of any of the recombinant nucleic acids, viruses, medicaments, and/or compositions or formulations described herein. In some embodiments, the subject is a human.

In some embodiments, localized delivery of an antibody using a recombinant nucleic acid, virus, medicament, and/or pharmaceutical composition or formulation described herein reduces or eliminates systemic exposure to the antibody in the subject, e.g., as compared to a subject who received treatment with a purified antibody delivered via a traditional route of antibody administration (such as intravenous or subcutaneous administration). Methods of measuring systemic exposure to an antibody are well known to one of ordinary skill in the art, including, for example, by measuring the quantity of the antibody present in the blood or serum of a subject by ELISA. In some embodiments, use of a recombinant nucleic acid, virus, medicament, and/or pharmaceutical composition or formulation described herein for localized delivery of an antibody to a subject reduces or eliminates one or more side effects of the antibody, as compared to the side effects observed after systemic exposure of the subject to the same antibody (e.g., comparing one or more side effects of expressing the antibody in the subject after delivery (e.g., topical, intraarticular, intravitreal, etc.) of a recombinant nucleic acid, virus, medicament, and/or pharmaceutical composition or formulation described herein vs. the side effects after systemic (e.g., intravenous or subcutaneous) administration of the purified antibody). Examples of side effects resulting from systemic exposure to a therapeutic antibody include, for example, allergic reactions, chills, weakness, diarrhea, nausea, vomiting, rash, itching, high blood glucose levels, cough, constipation, shortness of breath, peripheral edema, headache, fever, muscle aches and pains, decreased appetite, increased triglyceride levels, insomnia, abdominal pain, back pain, dizziness, low blood pressure, anaphylaxis, infections, cancer, serum sickness, autoimmune thyroiditis, arterial and venous blood clots, congestive heart failure, bleeding, interstitial lung disease, hepatitis, gastrointestinal perforation, enterocolitis, mucositis, stomatitis, anemia, reduced white blood cell count, and/or hypothyroidism. Methods of assessing antibody side effects are well known to one of ordinary skill in the art.

In some embodiments, use of a recombinant nucleic acid, virus, medicament, and/or pharmaceutical composition or formulation described herein for localized delivery of an antibody to a subject improves one or more pharmacokinetic properties of the antibody at the site of interest (e.g., in the skin, in a joint, in the eye, etc.), as compared the pharmacokinetic properties of the antibody at the site of interest after systemic (e.g., intravenous) administration of the purified antibody. For a review of antibody pharmacokinetic properties, see e.g., Ryman and Meibohm “Pharmacokinetics of Monoclonal Antibodies”, CPT Pharmacometrics Syst Pharmacol. 2017 September; 6(9):576-588.

In some embodiments, use of a recombinant nucleic acid, virus, medicament, and/or pharmaceutical composition or formulation described herein for localized delivery of an antibody to a subject increases antibody tissue accessibility and/or infiltration at a site of interest (e.g., in the skin, in a joint, in the eye, in the airway or lungs, etc.), as compared to antibody tissue accessibility and/or infiltration at the site of interest after systemic (e.g., intravenous or subcutaneous) administration of the purified antibody. In some embodiments, use of a recombinant nucleic acid, virus, medicament, and/or pharmaceutical composition or formulation described herein for localized delivery of an antibody to a subject increases antibody concentration at the site of interest (e.g., in the skin, in a joint, in the eye, in the airway or lungs, etc.), as compared to antibody concentration at the site of interest after systemic (e.g., intravenous or subcutaneous) administration of the purified antibody. For example, in some embodiments, use of a recombinant nucleic acid, virus, medicament, and/or pharmaceutical composition or formulation described herein for localized delivery of an antibody increases antibody concentration at a site of interest (e.g., in the skin, in a joint, in the eye, in the airway or lungs, etc.) by at least about 25%, at least about 30%, at least about 40%, at least about 50%, at least about 50%, at least about 60%, at least about 70%, at least about 80%, at least about 90%, at least about 95%, at least about 99%, or more, as compared to antibody concentration at the site of interest after systemic (e.g., intravenous or subcutaneous) administration of the purified antibody. In some embodiments, use of a recombinant nucleic acid, virus, medicament, and/or pharmaceutical composition or formulation described herein for localized delivery of an antibody increases antibody concentration at a site of interest (e.g., in the skin, in a joint, in the eye, in the airway or lungs, etc.) by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, or at least about 1000-fold, as compared to antibody concentration at the site of interest after systemic (e.g., intravenous or subcutaneous) administration of the purified antibody. Methods of measuring antibody concentration in a tissue sample are readily available to one of ordinary skill in the art, including, for example, by western blots, ELISAs, immunofluorescence, mass spectrometry, etc.

Other aspects of the present disclosure relate to methods of providing prophylactic, palliative, and/or therapeutic relief of one or more signs or symptoms of a disease in a subject comprising administering to the subject an effective amount of any of the recombinant nucleic acids, viruses, medicaments, and/or pharmaceutical compositions or formulations described herein. In some embodiments, the subject is a human. The disease may be any disease known in the art that may benefit from treatment with a therapeutic antibody, including, for example, psoriasis (e.g., chronic plaque psoriasis), atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer (e.g., skin cancer, breast cancer, lymphoma, colorectal cancer, head and neck cancer, etc.), hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, autoimmune disease (e.g., multiple sclerosis, systemic lupus erythematosus, inflammatory bowel disease, Addison's disease, Graves' disease, Sjogren's syndrome, Hashimoto's thyroiditis, Myasthenia gravis, vasculitis, pernicious anemia, celiac disease, etc.), asthma, uveal melanoma, thyroid eye disease, infectious diseases, graft/tissue/organ rejection, neurological diseases (e.g., Alzheimer's), etc. In some embodiments, the disease not cancer.

The recombinant nucleic acids, viruses, medicaments, and/or pharmaceutical compositions or formulations described herein may be administered by any suitable method or route known in the art, including, without limitation, by oral administration, sublingual administration, buccal administration, intranasal administration, intratracheal administration, topical administration, rectal administration, via inhalation, transdermal administration, subcutaneous injection, intradermal injection, intravenous (IV) injection, intra-arterial injection, intramuscular injection, intracardiac injection, intraosseous injection, intraperitoneal injection, transmucosal administration, vaginal administration, intravitreal administration, intraorbital administration, subconjunctival administration (e.g., the use of subconjunctival depots), suprachoroidal administration, subretinal administration, intra-articular administration, peri-articular administration, local administration, epicutaneous administration, or any combinations thereof. The present disclosure thus encompasses methods of delivering any of the recombinant nucleic acids, viruses, medicaments, and/or pharmaceutical compositions or formulations described herein to an individual (or a specific site or tissue thereof).

In some embodiments, the recombinant nucleic acid, virus, medicament, and/or pharmaceutical composition or formulation used in the methods of the present disclosure is administered cutaneously, topically, transdermally, subcutaneously, intradermally, transmucosally, sublingually, nasally, buccally, intranasally, intratracheally, intravitreally, subconjunctivally, suprachoroidally, subretinally, intraarticularly, or via inhalation to the subject. In some embodiments, the recombinant nucleic acid, virus, medicament, and/or pharmaceutical composition or formulation is administered topically to the subject. In some embodiments, the recombinant nucleic acid, virus, medicament, and/or pharmaceutical composition or formulation is administered intradermally to the subject. In some embodiments, the recombinant nucleic acid, virus, medicament, and/or pharmaceutical composition or formulation is administered orally, sublingually, buccally, nasally, intranasally, intratracheally, or via inhalation to the subject. In some embodiments, the recombinant nucleic acid, virus, medicament, and/or pharmaceutical composition or formulation is administered intraarticularly to the subject. In some embodiments, the recombinant nucleic acid, virus, medicament, and/or pharmaceutical composition or formulation is administered intraorbitally, intravitreally, subconjunctivally, suprachoroidally, subretinally, or topically (to the eye) of the subject.

In some embodiments, the recombinant nucleic acid, virus, medicament, and/or pharmaceutical composition or formulation is administered once to the subject. In some embodiments, the recombinant nucleic acid, virus, medicament, and/or pharmaceutical composition or formulation is administered at least twice (e.g., at least 2 times, at least 3 times, at least 4 times, at least 5 times, at least 10 times, etc.) to the subject. In some embodiments, at least about 1 hour (e.g., at least about 1 hour, at least about 6 hours, at least about 12 hours, at least about 18 hours, at least about 1 day, at least about 2 days, at least about 3 days, at least about 4 days, at least about 5 days, at least about 6 days, at least about 7 days, at least about 15 days, at least about 20 days, at least about 30 days, at least about 40 days, at least about 50 days, at least about 60 days, at least about 70 days, at least about 80 days, at least about 90 days, at least about 100 days, at least about 120 days, etc.) pass between administrations (e.g., between the first and second administrations, between the second and third administrations, etc.). In some embodiments, the recombinant nucleic acid, virus, medicament, and/or pharmaceutical composition or formulation is administered one, two, three, four, five or more times per day to the subject. In some embodiments, the recombinant nucleic acid, virus, medicament and/or pharmaceutical composition or formulation is administered to one or more affected and/or unaffected areas of the subject.

Other aspects of the present disclosure relate to a method of administering an antibody to the epidermis and/or dermis of a subject comprising topically, transdermally, and/or intradermally administering an effective amount of any of the recombinant nucleic acids, viruses, medicaments, and/or pharmaceutical compositions or formulations described herein to the subject. In some embodiments, the subject is not exposed to the antibody systemically (e.g., it is not detectable in the serum). In some embodiments, the recombinant nucleic acid, virus, medicament, and/or pharmaceutical composition or formulation is administered topically. In some embodiments, the recombinant nucleic acid, virus, medicament, and/or pharmaceutical composition or formulation is administered intradermally. In some embodiments, the subject is a human. In some embodiments, the subject suffers from a disease or disorder of the skin. In some embodiments, the subject suffers from one or more of psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, skin cancer, and/or hidradenitis suppurativa. In some embodiments, the disease or disorder is not cancer (e.g., is not skin cancer).

In some embodiments, one or more portions of the skin of the subject is abraded or made more permeable prior to treatment with a recombinant nucleic acid, virus, medicament, and/or pharmaceutical composition or formulation described herein. Any suitable method of abrading the skin or increasing skin permeability known in the art may be used, including, for example, use of a dermal roller, repeated use of adhesive strips to remove layers of skin cells (tape stripping), scraping with a scalpel or blade, use of sandpaper, use of chemical permeation enhancers (e.g., cell-penetrating polypeptides) or electrical energy, use of sonic or ultrasonic energy, use of light (e.g., laser) energy, use of micron-sized needles or blades with a length suitable to pierce but not completely pass through the epidermis, etc.

Other aspects of the present disclosure relate to a method of administering an antibody to the mucosa of a subject comprising topically, transmucosally, orally, sublingually, nasally, intranasally, intratracheally, via inhalation, or buccally administering an effective amount of any of the recombinant nucleic acids, viruses, medicaments, and/or pharmaceutical compositions or formulations described herein to the subject. In some embodiments, the subject is not exposed to the antibody systemically (e.g., it is not detectable in the serum). In some embodiments, the recombinant nucleic acid, virus, medicament, and/or composition is administered sublingually. In some embodiments, the recombinant nucleic acid, virus, medicament, and/or pharmaceutical composition or formulation is administered topically. In some embodiments, the recombinant nucleic acid, virus, medicament, and/or pharmaceutical composition or formulation is administered buccally. In some embodiments, the recombinant nucleic acid, virus, medicament, and/or pharmaceutical composition or formulation is administered intranasally. In some embodiments, the recombinant nucleic acid, virus, medicament, and/or pharmaceutical composition or formulation is administered via inhalation. In some embodiments, the subject is a human.

Other aspects of the present disclosure relate to a method of administering an antibody to the airway or lungs of a subject comprising orally, sublingually, nasally, intranasally, intratracheally, via inhalation, or buccally administering an effective amount of any of the recombinant nucleic acids, viruses, medicaments, and/or pharmaceutical compositions or formulations described herein to the subject. In some embodiments, the subject is not exposed to the antibody systemically (e.g., it is not detectable in the serum). In some embodiments, the recombinant nucleic acid, virus, medicament, and/or composition is administered sublingually. In some embodiments, the recombinant nucleic acid, virus, medicament, and/or pharmaceutical composition or formulation is administered buccally. In some embodiments, the recombinant nucleic acid, virus, medicament, and/or pharmaceutical composition or formulation is administered intranasally. In some embodiments, the recombinant nucleic acid, virus, medicament, and/or pharmaceutical composition or formulation is administered via inhalation. In some embodiments, the subject is a human. In some embodiments, the subject suffers from a disease or disorder of the airway or lungs (e.g., a respiratory disease such as asthma, lung cancer, respiratory infections, chronic obstructive pulmonary disease, idiopathic pulmonary fibrosis, etc.).

Other aspects of the present disclosure relate to a method of administering an antibody to one or more joints of a subject comprising intraarticularly administering an effective amount of any of the recombinant nucleic acids, viruses, medicaments, and/or pharmaceutical compositions or formulations described herein to the subject. In some embodiments, the subject is not exposed to the antibody systemically (e.g., it is not detectable in the serum). In some embodiments, the subject suffers from a disease of the joints. In some embodiments, the subject suffers from one or more of arthritis, rheumatoid arthritis, psoriatic arthritis, osteoarthritis, juvenile idiopathic arthritis, ankylosing spondylitis, axial spondylarthritis, reactive arthritis, and/or enteropathic arthritis. In some embodiments, the subject is a human.

Other aspects of the present disclosure relate to a method of administering an antibody to one or both eyes of a subject comprising topically, intravitreally, intraorbitally, subconjunctivally, subretinally, or suprachoroidally administering an effective amount of any of the recombinant nucleic acids, viruses, medicaments, and/or pharmaceutical compositions or formulations described herein to the subject. In some embodiments, the subject is not exposed to the antibody systemically (e.g., it is not detectable in the serum). In some embodiments, the subject suffers from a disease of the eye. In some embodiments, the subject suffers from an autoimmune disease that effects the eyes. In some embodiments, the subject suffers from uveal melanoma or thyroid eye disease. In some embodiments, the subject is a human.

### VII. Host Cells

Certain aspects of the present disclosure relate to one or more host cells comprising any of the recombinant nucleic acids described herein. Any suitable host cell (prokaryotic or eukaryotic) known in the art may be used, including, for example: prokaryotic cells including eubacteria, such as Gram-negative or Gram-positive organisms, for example Enterobacteriaceae such as Escherichia (e.g., E. coli), Enterobacter, Erminia, Klebsiella, Proteus, Salmonella (e.g., S. typhimurium), Serratia (e.g., S. marcescans), and Shigella, as well as Bacilli such as B. subtilis and B. licheniformis; fungal cells (e.g., S. cerevisiae); insect cells (e.g., S2 cells, etc.); and mammalian cells, including monkey kidney CV1 line transformed by SV40 (COS-7, ATCC CRL 1651), human embryonic kidney line (293 or 293 cells subcloned for growth in suspension culture), baby hamster kidney cells (BHK, ATCC CCL 10), mouse Sertoli cells (TM4), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells, MRC 5 cells, FS4 cells, human hepatoma line (Hep G2), Chinese hamster ovary (CHO) cells, including DHFR″ CHO cells, and myeloma cell lines such as NS0 and Sp2/0. In some embodiments, the host cell is a human or non-human primate cell. In some embodiments, the host cells are cells from a cell line. Examples of suitable host cells or cell lines may include, but are not limited to, 293, HeLa, SH-Sy5y, Hep G2, CACO-2, A549, L929, 3T3, K562, CHO-K1, MDCK, HUVEC, Vero, N20, COS-7, PSN1, VCaP, CHO cells, and the like.

In some embodiments, the recombinant nucleic acid is a herpes simplex viral vector. In some embodiments, the recombinant nucleic acid is a herpes simplex virus amplicon. In some embodiments, the recombinant nucleic acid is an HSV-1 amplicon or HSV-1 hybrid amplicon. In some embodiments, a host cell comprising a helper virus is contacted with an HSV-1 amplicon or HSV-1 hybrid amplicon described herein, resulting in the production of a virus comprising one or more recombinant nucleic acids described herein. In some embodiments, the virus is collected from the supernatant of the contacted host cell. Methods of generating virus by contacting host cells comprising a helper virus with an HSV-1 amplicon or HSV-1 hybrid amplicon are known in the art

In some embodiments, the host cell is a complementing host cell. In some embodiments, the complementing host cell expresses one or more genes that are inactivated in any of the viral vectors described herein. In some embodiments, the complementing host cell is contacted with a recombinant herpes viral genome (e.g., a recombinant herpes simplex viral genome) described herein. In some embodiments, contacting a complementing host cell with a recombinant herpes virus genome results in the production of a herpes virus comprising one or more recombinant nucleic acids described herein. In some embodiments, the virus is collected from the supernatant of the contacted host cell. Methods of generating virus by contacting complementing host cells with a recombinant herpes simplex virus are generally described in WO2015/009952 and/or WO2017/176336.

### VIII. Articles of Manufacture or Kits

Certain aspects of the present disclosure relate to an article of manufacture or a kit comprising any of the recombinant nucleic acids, viruses, medicaments, and/or pharmaceutical compositions or formulations) described herein. In some embodiments, the article of manufacture or kit comprises a package insert comprising instructions for administering the recombinant nucleic acid, virus, medicament, and/or pharmaceutical composition or formulation (e.g., to provide a method of locally delivering an antibody to one or more tissues of the subject (such as the epidermis and/or dermis of a subject) in need thereof by administering the recombinant nucleic acid, virus, medicament, and/or pharmaceutical composition or formulation).

Suitable containers for the recombinant nucleic acids, viruses, medicaments and/or pharmaceutical compositions or formulations may include, for example, bottles, vials, bags, tubes, and syringes. The container may be formed from a variety of materials such as glass, plastic (such as polyvinyl chloride or polyolefin), or metal alloy (such as stainless steel or hastelloy). In some embodiments, the container comprises a label on, or associated with the container, wherein the label indicates directions for use. The article of manufacture or kit may further include other materials desirable from a commercial and user standpoint, including other buffers, diluents, filters, needles, syringes, package inserts, and the like.

### IX. Enumerated Embodiments

Embodiment 1: a recombinant herpes simplex virus (HSV) genome comprising one or more polynucleotides encoding an antibody.

Embodiment 2: the recombinant genome of embodiment 1, wherein the antibody is an antibody fragment.

Embodiment 3: the recombinant genome of embodiment 2, wherein the antibody fragment is a Fab, Fab′, Fab′-SF, F(ab′)2, Fv, scFv, or scFv-Fc fragment.

Embodiment 4: the recombinant genome of embodiment 1, wherein the antibody is a full-length antibody.

Embodiment 5: the recombinant genome of any one of embodiments 1-4, wherein the antibody is a murine antibody, a chimeric antibody, a humanized antibody, a human antibody, a monoclonal antibody, or a multispecific antibody.

Embodiment 6: the recombinant genome of any one of embodiments 1-5, wherein the antibody is an IgA, IgD, IgE, IgG, or IgM antibody.

Embodiment 7: the recombinant genome of any one of embodiments 1-6, wherein the antibody is an IgG antibody.

Embodiment 8: the recombinant genome of embodiment 7, wherein the IgG antibody is an IgG1, IgG2, IgG3, or IgG4 antibody.

Embodiment 9: the recombinant genome of any one of embodiments 1-8, wherein the antibody is an agonist antibody.

Embodiment 10: the recombinant genome of any one of embodiments 1-8, wherein the antibody is an antagonist antibody.

Embodiment 11: the recombinant genome of any one of embodiments 1-10, wherein the antibody comprises a heavy chain variable region comprising an HVR-H1, an HVR-H2, and an HVR-H3, wherein the HVR-H1 comprises a sequence selected from the group consisting of SEQ ID NOS: 1-59, the HVR-H2 comprises a sequence selected from the group consisting of SEQ ID NOS: 60-122, and/or the HVR-H3 comprises a sequence selected from the group consisting of SEQ ID NOS: 123-185.

Embodiment 12: the recombinant genome of embodiment 11, wherein the heavy chain variable region comprises a sequence having at least 85%, at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOS: 355-419.

Embodiment 13: the recombinant genome of any one of embodiments 1-12, wherein the antibody comprises a light chain variable region comprising an HVR-L1, an HVR-L2, and an HVR-L3, wherein the HVR-L1 comprises a sequence selected from the group consisting of SEQ ID NOS: 186-242, the HVR-L2 comprises a sequence selected from the group consisting of SEQ ID NOS: 243-294, and/or the HVR-L3 comprises a sequence selected from the group consisting of SEQ ID NOS: 295-354.

Embodiment 14: the recombinant genome of embodiment 13, wherein the light chain variable region comprises a sequence having at least 85%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 420-482.

Embodiment 15: the recombinant genome of any one of embodiments 1-14, wherein the recombinant genome is a recombinant HSV-1 genome, a recombinant HSV-2 genome, or any derivatives thereof.

Embodiment 16: the recombinant genome of any one of embodiments 1-15, wherein the recombinant genome comprises an inactivating mutation in a herpes simplex virus gene.

Embodiment 17: the recombinant genome of embodiment 16, wherein the herpes simplex virus gene is selected from the group consisting of Infected Cell Protein (ICP) 0, ICP4, ICP22, ICP27, ICP47, thymidine kinase (tk), Long Unique Region (UL) 41, and UL55.

Embodiment 18: the recombinant genome of embodiment 17, wherein the recombinant genome comprises an inactivating mutation in one or both copies of the ICP4 gene.

Embodiment 19: the recombinant genome of embodiment 17 or 18, wherein the recombinant genome comprises an inactivating mutation in the ICP22 gene.

Embodiment 20: the recombinant genome of any one of embodiments 17-19, wherein the recombinant genome comprises an inactivating mutation in the UL41 gene.

Embodiment 21: the recombinant genome of any one of embodiments 17-20, wherein the recombinant genome comprises an inactivating mutation in the ICP0 gene.

Embodiment 22: the recombinant genome of any one of embodiments 17-21, wherein the recombinant genome comprises an inactivating mutation in the ICP27 gene.

Embodiment 23: the recombinant genome of any one of embodiments 16-22, wherein the inactivating mutation is a deletion of the coding sequence of the gene(s).

Embodiment 24: the recombinant genome of any one of embodiments 1-23, wherein the recombinant genome has reduced cytotoxicity when introduced into a target cell as compared to a wild-type herpes simplex virus genome.

Embodiment 25: the recombinant genome of embodiment 24, wherein the target cell is a human cell.

Embodiment 26: the recombinant genome of embodiment 24 or 25, wherein the target cell is a keratinocyte or fibroblast.

Embodiment 27: the recombinant genome of any one of embodiments 1-2, wherein the recombinant genome comprises the one or more polynucleotides within one or more viral gene loci.

Embodiment 28: the recombinant genome of embodiment 27, wherein the recombinant genome comprises one or more polynucleotides within one or both of the ICP4 viral gene loci.

Embodiment 29: the recombinant genome of embodiment 27 or 28, wherein the recombinant genome comprises the one or more polynucleotides within the ICP22 viral gene locus.

Embodiment 30: the recombinant genome of any one of embodiments 27-29, wherein the recombinant genome comprises the one or more polynucleotides within the UL41 viral gene locus.

Embodiment 31: a herpes simplex virus (HSV) comprising the recombinant genome of any one of embodiments 1-30.

Embodiment 32: the virus of embodiment 31, wherein the HSV is replication competent.

Embodiment 33: the virus of embodiment 31, wherein the HSV is replication defective.

Embodiment 34: the virus of any one of embodiments 31-33, wherein the HSV has reduced cytotoxicity as compared to a wild-type herpes simplex virus.

Embodiment 35: the virus of any one of embodiments 31-34, wherein the HSV is a herpes simplex type 1 virus, a herpes simplex type 2 virus, or any derivatives thereof.

Embodiment 36: a pharmaceutical composition comprising the recombinant genome of any one of embodiments 1-30 or the virus of any one of embodiments 31-35 and a pharmaceutically acceptable excipient.

Embodiment 37: the pharmaceutical composition of embodiment 36, wherein the pharmaceutically acceptable excipient is suitable for topical, transdermal, subcutaneous, intradermal, transmucosal, sublingual, nasal, buccal, intraorbital, intravitreal, subconjunctival, suprachoroidal, intraarticular, and/or inhaled administration.

Embodiment 38: the pharmaceutical composition of embodiment 36 or 37, wherein the pharmaceutically acceptable excipient is suitable for topical administration.

Embodiment 39: the pharmaceutical composition of any one of embodiments 36-38, wherein the pharmaceutically acceptable excipient comprises a hydroxypropyl methylcellulose gel.

Embodiment 40: the pharmaceutical composition of any one of embodiments 36-39, wherein the pharmaceutically acceptable excipient comprises a phosphate buffer.

Embodiment 41: the pharmaceutical composition of any one of embodiments 36-40, wherein the pharmaceutically acceptable excipient comprises glycerol.

Embodiment 42: the pharmaceutical composition of any one of embodiments 36-41, wherein the pharmaceutically acceptable excipient comprises a lipid carrier.

Embodiment 43: the pharmaceutical composition of any one of embodiments 36-42, wherein the pharmaceutically acceptable excipient comprises a nanoparticle carrier.

Embodiment 44: a method of administering an antibody to a subject, the method comprises administering to the subject an effective amount of the virus of any one of embodiments 31-35 or the pharmaceutical composition of any one of embodiments 36-43.

Embodiment 45: a method of providing prophylactic, palliative, and/or therapeutic relief of one or more signs or symptoms of a disease in a subject, the method comprising administering to the subject an effective amount of the virus of any one of embodiments 31-35 or the pharmaceutical composition of any one of embodiments 36-43.

Embodiment 46: the method of embodiment 44 or 45, wherein the virus or composition is administered topically, transdermally, subcutaneously, intradermally, transmucosally, sublingually, nasally, buccally, intravitreally, subconjunctivally, suprachoroidally, intraarticularly, or via inhalation.

Embodiment 47: the method of embodiment 45 or 46, wherein the disease is selected from the group consisting of psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer, hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, autoimmune disease, melanoma, uveal melanoma, and thyroid eye disease.

Embodiment 48: a method of administering an antibody to the epidermis and/or dermis of a subject, the method comprising topically transdermally or intradermally administering to the subject an effective amount of the virus of any one of embodiments 31-35 or the pharmaceutical composition of any one of embodiments 36-43.

Embodiment 49: the method of embodiment 48, wherein the skin of the subject is abraded prior to administration.

Embodiment 50: a method of administering an antibody to the mucosa of a subject, the method comprising topically, transmucosally, sublingually, nasally, or buccally administering to the subject an effective amount of the virus of any one of embodiments 31-35 or the pharmaceutical composition of any one of embodiments 36-43.

Embodiment 51: a method of administering an antibody to one or more joints of a subject, the method comprising intraarticularly administering to the subject an effective amount of the virus of any one of embodiments 31-35 or the pharmaceutical composition of any one of embodiments 36-43.

Embodiment 52: a method of administering an antibody to one or both eyes of a subject, the method comprising topically, intraorbitally, intravitreally, subconjunctivally, or suprachoroidally administering to the subject an effective amount of the virus of any one of embodiments 31-35 or the pharmaceutical composition of any one of embodiments 36-43.

Embodiment 53: the method of any one of embodiments 44-52, wherein the subject is a human.

Embodiment 54: the method of any one of embodiments 44-53, wherein the subject is not exposed to the antibody systemically.

The specification is considered to be sufficient to enable one skilled in the art to practice the present disclosure. Various modifications of the present disclosure in addition to those shown and described herein will become apparent to those skilled in the art from the foregoing description and fall within the scope of the appended claims.

### EXAMPLES

The present disclosure will be more fully understood by reference to the following examples. It should not, however, be construed as limiting the scope of the present disclosure. It is understood that the examples and embodiments described herein are for illustrative purposes only and that various modifications or changes in light thereof will be suggested to persons skilled in the art, and are to be included within the spirit and purview of this application and scope of the appended claims.

### Example 1: Modified Herpes Simplex Virus Vectors Encoding Antibodies

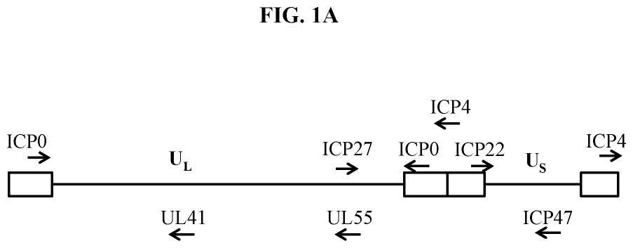

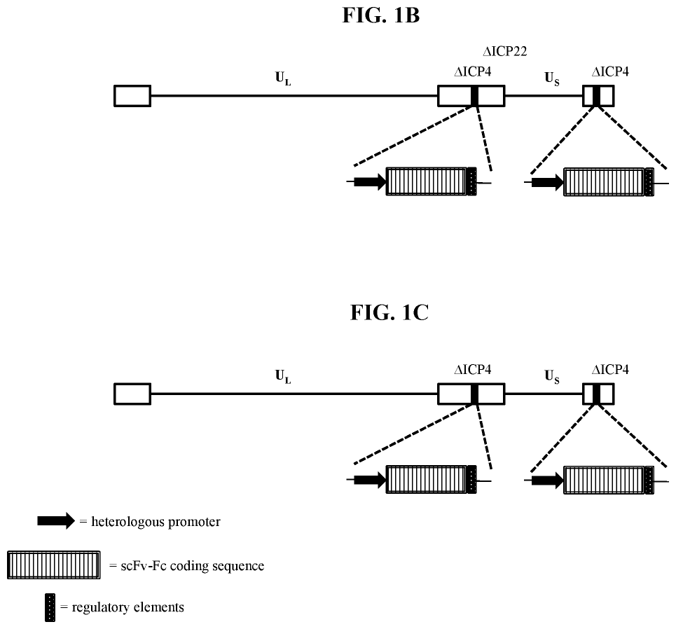

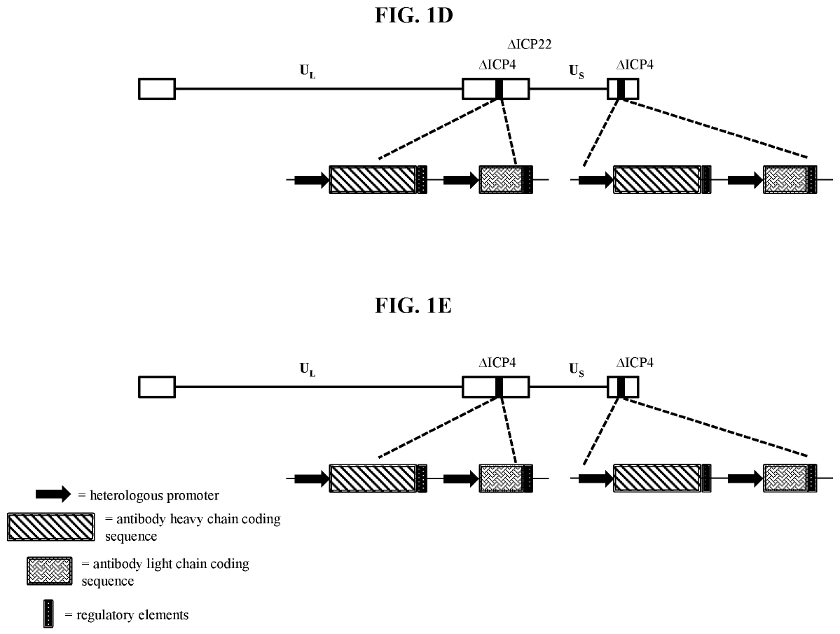

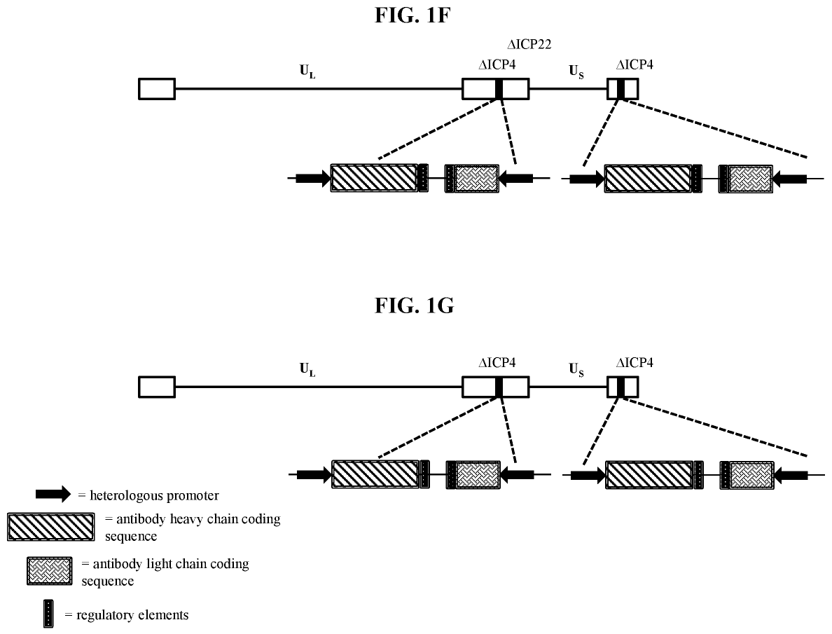

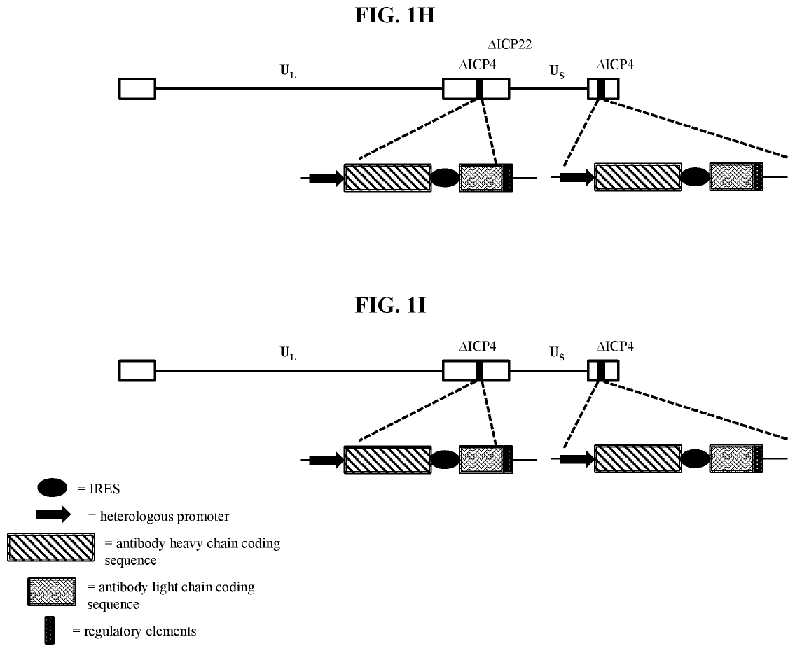

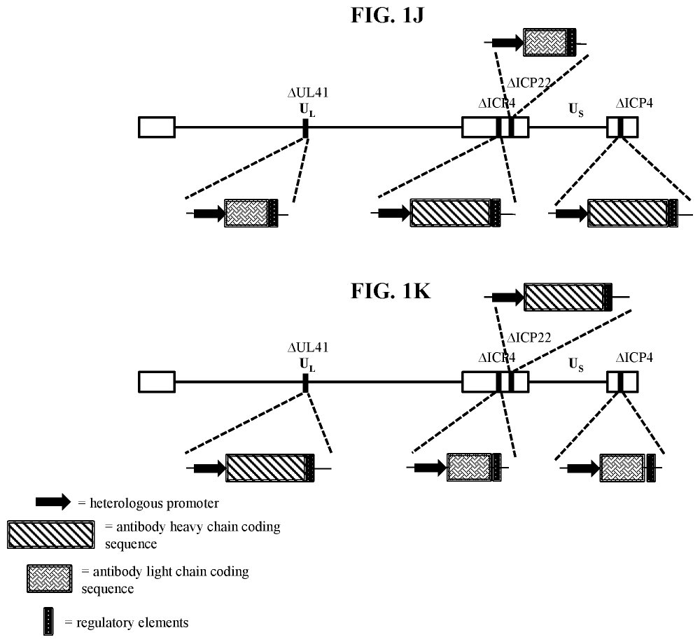

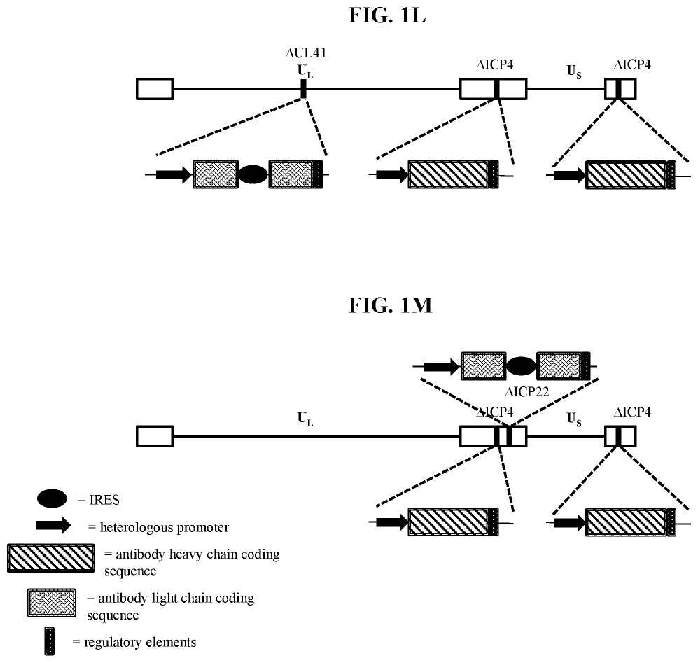

To make modified herpes simplex virus genome vectors capable of expressing antibodies in a target mammalian cell (such as a human keratinocyte or fibroblast), a herpes simplex virus genome (FIG. 1A) is first modified to inactivate one or more herpes simplex virus genes. Such modifications may decrease the toxicity of the genome in mammalian cells. Next, variants of these modified/attenuated recombinant viral constructs are generated such that they carry one or more polynucleotides encoding the desired antibody. These variants include: 1) a recombinant ΔICP4/ΔICP22-modified HSV-1 genome comprising expression cassettes containing the coding sequence of an antibody fragment (e.g., an scFv-Fc) under the control of a heterologous promoter integrated at each ICP4 locus (FIG. 1B); 2) a recombinant ΔICP4-modified HSV-1 genome comprising expression cassettes containing the coding sequence of an antibody fragment (e.g., an scFv-Fc) under the control of a heterologous promoter integrated at each ICP4 locus (FIG. 1C); 3) a recombinant ΔICP4/ΔICP22-modified HSV-1 genome comprising expression cassettes containing the coding sequence of an antibody heavy chain under the control of a first heterologous promoter and the coding sequence of an antibody light chain under the control of a second heterologous promoter on the same strand of DNA integrated at each ICP4 locus (FIG. 1D); 4) a recombinant ΔICP4-modified HSV-1 genome comprising expression cassettes containing the coding sequence of an antibody heavy chain under the control of a first heterologous promoter and the coding sequence of an antibody light chain under the control of a second heterologous promoter on the same strand of DNA integrated at each ICP4 locus (FIG. 1E); 5) a recombinant ΔICP4/ΔICP22-modified HSV-1 genome comprising expression cassettes containing the coding sequence of an antibody heavy chain under the control of a first heterologous promoter and the coding sequence of an antibody light chain under the control of a second heterologous promoter on opposite strands of DNA integrated at each ICP4 locus (FIG. 1F); 6) a recombinant ΔICP4-modified HSV-1 genome comprising expression cassettes containing the coding sequence of an antibody heavy chain under the control of a first heterologous promoter and the coding sequence of an antibody light chain under the control of a second heterologous promoter on opposite strands of DNA integrated at each ICP4 locus (FIG. 1G); 7) a recombinant ΔICP4/ΔICP22-modified HSV-1 genome comprising expression cassettes encoding a polycistronic mRNA under the control of a heterologous promoter integrated at each of the ICP4 loci, where the polycistronic mRNA contains the coding sequence of an antibody heavy and light chain separated by an internal ribosomal entry site (IRES) (FIG. 1H); 8) a recombinant ΔICP4-modified HSV-1 genome comprising expression cassettes encoding a polycistronic mRNA under the control of a heterologous promoter integrated at each of the ICP4 loci, where the polycistronic mRNA contains the coding sequence of an antibody heavy and light chain separated by an internal ribosomal entry site (IRES) (FIG. 1I); 9) a recombinant ΔICP4/ΔICP22/ΔUL41-modified HSV-1 genome comprising expression cassettes containing the coding sequence of an antibody heavy chain under the control of a heterologous promoter integrated at each of the ICP4 loci, and an expression cassette containing the coding sequence of an antibody light chain under the control of a heterologous promoter integrated at the UL41 and ICP22 loci (FIG. 1J); 10) a recombinant ΔICP4/ΔICP22/ΔUL41-modified HSV-1 genome comprising expression cassettes containing the coding sequence of an antibody light chain under the control of a heterologous promoter integrated at each of the ICP4 loci, and an expression cassette containing the coding sequence of an antibody heavy chain under the control of a heterologous promoter integrated at the UL41 and ICP22 loci (FIG. 1K); 11) a recombinant ΔICP4/ΔUL41-modified HSV-1 genome comprising expression cassettes containing the coding sequence of an antibody heavy chain under the control of a heterologous promoter integrated at each of the ICP4 loci, and an expression cassette containing the coding sequence of a polycistronic mRNA under the control of a heterologous promoter integrated at the UL41 locus, where the polycistronic mRNA contains two copies of the coding sequence of an antibody light chain separated by an internal ribosomal entry site (IRES) (FIG. 1L); 12) a recombinant ΔICP4/ΔICP22-modified HSV-1 genome comprising expression cassettes containing the coding sequence of an antibody heavy chain under the control of a heterologous promoter integrated at each of the ICP4 loci, and an expression cassette containing the coding sequence of a polycistronic mRNA under the control of a heterologous promoter integrated at the ICP22 locus, where the polycistronic mRNA contains two copies of the coding sequence of an antibody light chain separated by an internal ribosomal entry site (IRES) (FIG. 1M); and 14) a recombinant ΔICP4/ΔICP22/ΔUL41-modified HSV-1 genome comprising expression cassettes containing the coding sequence of an antibody light chain under the control of a heterologous promoter integrated at the UL41 locus, and an expression cassette containing the coding sequence of an antibody heavy chain under the control of a heterologous promoter integrated at the ICP22 locus (FIG. 1N). The modified herpes simplex virus genome may be engineered as described above, except that the coding sequence for the full-length antibody heavy chain is replaced with the coding sequence of an antibody heavy chain variable region and constant region 1 (CH₁), such that the recombinant herpes simplex virus produces a Fab fragment.

These modified herpes simplex virus genome vectors are transfected into engineered Vero cells that are modified to express one or more herpes virus genes. These engineered Vero cells secrete in the supernatant of the cell culture a replication defective herpes simplex virus with the modified genomes packaged therein. The supernatant is then collected, concentrated, and sterile filtered through a 5 μm filter.

### Example 2: Construction and Validation of Recombinant Herpes Viruses Encoding Antibodies

First, a recombinant, attenuated HSV vector was designed to express a full-length antibody. A polynucleotide construct was generated which encoded, from 5′ to 3′, a first ORF encoding the antibody light chain (with a leader sequence), a synthetic IRES, and second ORF encoding the antibody heavy chain (with a leader sequence) (e.g., as depicted in FIGS. 1H and 1I). The IRES-based construct (encoded in an expression cassette which further contained a heterologous promoter and a polyA sequence) was then inserted into both copies of the HSV1 ICP4 gene locus. This first viral construct (HSV-flAb2) was engineered to express a chimeric anti-human CD20 IgG1 antibody containing a heavy chain variable region comprising the amino acid sequence of SEQ ID NO: 356 and a light chain variable region comprising the amino acid sequence of SEQ ID NO: 421.

Multiple plaques identified in a lawn of transgenic cells were picked and screened to identify vectors with correctly inserted IRES constructs. Briefly, Vero cells were infected with putative IRES viral isolates, infections were allowed to proceed for five days, and cell supernatants were harvested and tested for the presence of secreted full-length antibody by ELISA according to the manufacturer's instructions (Abcam, cat. no. ab195215). All of the tested isolates expressed and secreted their encoded antibody into the cell supernatants; however, the antibodies were produced at relatively low levels. The most productive isolate for HSV-flAb2 secreted 0.844 ng/mL of the encoded full-length chimeric antibody into the supernatant of infected Vero cells.

As an alternative to expressing full-length antibodies from the engineered vectors, which required the use of multiple ORFs encoding antibody light and heavy chains, recombinant HSV1 vectors were constructed to express single-chain antibodies from a single ORF (see e.g., FIGS. 1B and 1C). Here, single-chain antibodies were constructed to contain a leader sequence, a heavy chain variable region sequence, a light chain variable region sequence, a linker polypeptide linking the heavy and light chain variable regions, and an Fc region (i.e., an scFv-Fc antibody). Two different variants of each single-chain antibody were designed to contain both possible relative orientations of the light and heavy chain variable region sequences: the “Fc1” variant of each antibody contained, from n-terminus to c-terminus, a leader sequence-a heavy chain variable region-a linker sequence-a light chain variable region-and an Fc region; the “Fc2” variant of each antibody contained, from n-terminus to c-terminus, a leader sequence-a light chain variable region-a linker sequence-a heavy chain variable region-and an Fc region. These single-chain antibody constructs (encoded in an expression cassette which further contained a heterologous promoter and a polyA sequence) were then inserted into both copies of the HSV1 ICP4 gene locus. A summary of the sequences employed in each scFv-Fc antibody tested is provided in Table 4 below.

TABLE 4
HSV-encoded scFv-Fc antibodies

| | | | Heavy chain | Light chain | | |
|---|---|---|---|---|---|---|
| | | | variable | variable | | |
| Antibody | | | region | region | | |
| name: | Target: | Leader: | (VH): | (VL): | Linker: | Fc region: |
| Ab1Fc1 | Human | SEQ ID | SEQ ID | SEQ ID | SEQ ID | Human IgG1 Fc |
| | TNFα | NO: 510 | NO: 355 | NO: 420 | NO: 568 | (SEQ ID NO: 1120) |
| Ab1Fc2 | Human | SEQ ID | SEQ ID | SEQ ID | SEQ ID | Human IgG1 Fc |
| | TNFα | NO: 514 | NO: 355 | NO: 420 | NO: 568 | (SEQ ID NO: 1120) |
| Ab2Fc2 | Human | SEQ ID | SEQ ID | SEQ ID | SEQ ID | Human IgG1 Fc |
| | CD20 | NO: 514 | NO: 356 | NO: 421 | NO: 568 | (SEQ ID NO: 1120) |
| Ab5Fc1 | Human | SEQ ID | SEQ ID | SEQ ID | SEQ ID | Human IgG1 Fc |
| | IL-17 | NO: 488 | NO: 359 | NO: 424 | NO: 568 | (SEQ ID NO: 1120) |
| Ab5Fc2 | Human | SEQ ID | SEQ ID | SEQ ID | SEQ ID | Human IgG1 Fc |
| | IL-17 | NO: 536 | NO: 359 | NO: 424 | NO: 568 | (SEQ ID NO: 1120) |
| Ab37Fc1 | Human | SEQ ID | SEQ ID | SEQ ID | SEQ ID | Human IgG4 Fc |
| | IL-4Ra | NO: 510 | NO: 391 | NO: 456 | NO: 568 | (SEQ ID NO: 1121) |
| Ab37Fc2 | Human | SEQ ID | SEQ ID | SEQ ID | SEQ ID | Human IgG4 Fc |
| | IL-4Ra | NO: 536 | NO: 391 | NO: 456 | NO: 568 | (SEQ ID NO: 1121) |
| Ab66Fc1 | Mouse | SEQ ID | SEQ ID | SEQ ID | SEQ ID | Mouse IgG1 Fc |
| | IL-4Ra | NO: 510 | NO: 614 | NO: 866 | NO: 568 | (SEQ ID NO: 1122) |
| Ab66Fc2 | Mouse | SEQ ID | SEQ ID | SEQ ID | SEQ ID | Mouse IgG1 Fc |
| | IL-4Ra | NO: 1119 | NO: 614 | NO: 866 | NO: 568 | (SEQ ID NO: 1122) |
| Ab67Fc2 | Mouse | SEQ ID | SEQ ID | SEQ ID | SEQ ID | Human IgG1 Fc |
| | IL-17 | NO: 523 | NO: 615 | NO: 867 | NO: 568 | (SEQ ID NO: 1120) |
| Ab68Fc1 | Human | SEQ ID | SEQ ID | SEQ ID | SEQ ID | Human IgG1 Fc |
| | CCR4 | NO: 488 | NO: 616 | NO: 868 | NO: 568 | (SEQ ID NO: 1120) |
| Ab68Fc2 | Human | SEQ ID | SEQ ID | SEQ ID | SEQ ID | Human IgG1 Fc |
| | CCR4 | NO: 551 | NO: 616 | NO: 868 | NO: 568 | (SEQ ID NO: 1120) |

Virus plaques were identified and picked from lawns of engineered cells, and the isolates were individually screened in Vero cells to identify vectors that produced the encoded antibodies, as described above. Cell supernatants were harvested from the infected Vero cells and tested for the presence of secreted antibody by ELISA, according to the manufacturer's instructions (Abcam, cat. no. ab195215 for single-chain antibodies containing human Fc sequences; Abcam, cat. no. ab151276 for single-chain antibodies containing mouse Fc sequences). Table 5 below provides the average calculated concentration of each antibody secreted into the supernatant from infected cells.

TABLE 5
Concentration of single-chain antibodies
secreted from HSV-infected cells

| | | Concentration in Cell |
|---|---|---|
| Antibody name: | Target: | Supernatant (ng/mL): |
| Ab1Fc1 | Human TNFα | 5844.3724 |
| Ab1Fc2 | Human TNFα | 2880.65185 |
| Ab2Fc2 | Human CD20 | 16132.458 |
| Ab5Fc1 | Human IL-17 | >750 |
| Ab5Fc2 | Human IL-17 | >750 |
| Ab37Fc1 | Human IL-4Ra | 495.7056639 |
| Ab37Fc2 | Human IL-4Ra | >750 |
| Ab66Fc1 | Mouse IL-4Ra | 366.1442 |
| Ab66Fc2 | Mouse IL-4Ra | 357.26944 |
| Ab67Fc2 | Mouse IL-17 | >750 |
| Ab68Fc1 | Human CCR4 | >750 |
| Ab68Fc2 | Human CCR4 | >750 |

Surprisingly, all of the single-chain antibodies were secreted into the cell supernatant at a much higher concentration (at least >800-fold) than the full-length HSV-flAb2 antibody. As a direct comparison, the Fc2 single-chain variant of HSV-Ab2 was detected at a concentration 19,114-fold higher than the full-length variant of HSV-Ab2.

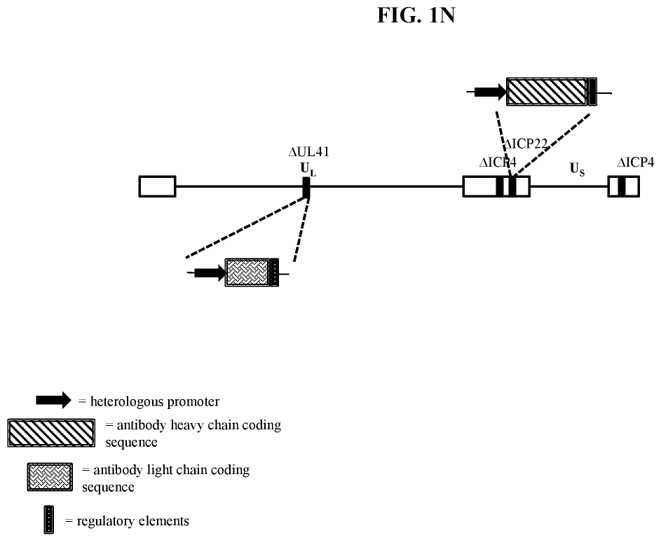

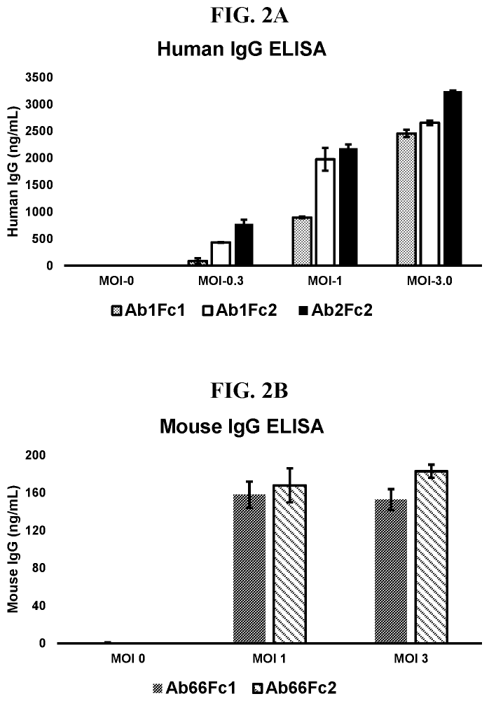

Next, a dose-ranging study was conducted to measure antibody secretion from human cells infected with engineered HSV1 vector encoding single-chain antibodies. Immortalized human keratinocytes (HaCaTs) were either mock infected (MOI 0) or infected with HSV vectors at various multiplicities of infection (MOIs) for 48 hours. The antibody-encoding vectors used in this experiment were: HSV-Ab1Fc1 (a single-chain human anti-human TNFα antibody), HSV-Ab1Fc2 (a single-chain human anti-human TNFα antibody), HSV-Ab2Fc2 (a single-chain chimeric anti-human CD20 antibody), HSV-Ab66Fc1 (a single-chain mouse anti-mouse IL-4Ra antibody), and HSV-Ab66Fc2 (a single-chain mouse anti-mouse IL-4Ra antibody). Upon completion of infection, supernatants were harvested from the infected cells and centrifuged at 11,000×g for 5 minutes. The antibodies secreted into the clarified supernatants were then quantified using an anti-human IgG (FIG. 2A) ELISA kit (Abcam, cat. no. ab195215) or an anti-mouse IgG (FIG. 2B) ELISA kit (Abcam, cat. no. ab151276). While little-to-know antibody was detected from mock infected cells, secreted human and chimeric antibodies were robustly detected in a dose-dependent manner from infected human keratinocytes (FIG. 2A). Single-chain mouse antibodies were also detected from secreted human keratinocytes, though their secretion did not appear to be dose-dependent (FIG. 2B).

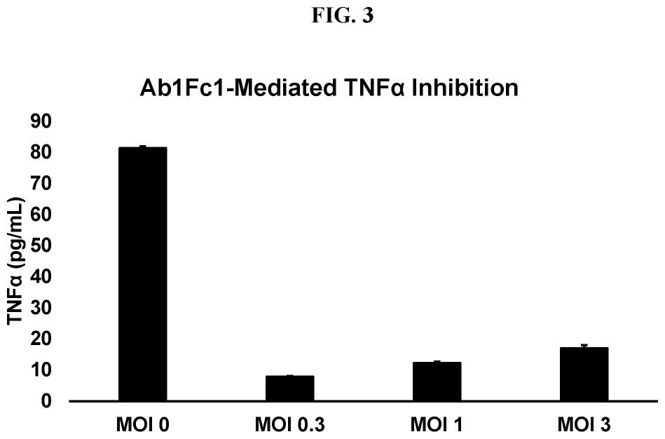

Finally, to confirm that the HSV-encoded antibodies were functional, the ability of HSV-Ab1Fc1, a single-chain human anti-human TNFα antagonist antibody, to inhibit ELISA-based detection of TNFα was examined Immortalized human keratinocytes (HaCaTs) were left uninfected or were infected with HSV-Ab1Fc1 at an MOI of 0.3, 1, and 3 for 48 hours. After completion of infection, cell supernatants were harvested and cleared by centrifugation at 11,000×6 for 5 minutes. Recombinant human TNFα (1000 pg/mL) was added into the clarified cell supernatants and incubated for 2 hours at 37° C. An anti-TNFα ELISA was then performed (Abcam, cat. no. ab181421) to determine whether the single-chain Ab1Fc1 produced by the engineered virus could mediate suppression of TNFα detection. The detectable level of TNFα was significantly reduced in cell supernatants harvested from infected human cells, as compared to mock-infected cells (FIG. 3), confirming that the secreted single-chain antibody was indeed functional.

Taken together, the data presented in this example indicated that: (1) recombinant HSV-1 vectors were successfully constructed to encode full-length antibodies as well as antibody fragments; (2) the engineered vectors expressed/secreted hundreds to thousands-fold higher levels of single-chain antibodies compared to full-length antibodies; (3) vectors could reliably express fully human, chimeric, and mouse antibodies at comparable levels (containing Fc regions from multiple IgG isotypes); (4) HSV-mediated antibody secretion from human cells was dose-dependent; and (5) the secreted antibodies were functional. Without wishing to be bound by theory, it is believed that engineered herpes viral vectors present a novel method of administering multiple types of antibodies to humans.

### Example 3: Herpes Virus-Encoded Antibodies in a Mouse Atopic Dermatitis (AD) Model

Atopic dermatitis is a chronic, relapsing, and often intensely pruritic inflammatory disorder of the skin. The main cause of the disease appears to be a defect of the epidermal barrier resulting from a combination of a genetic predisposition, functioning of the immune system, and environmental factors. While the treatment for moderate-to-severe AD has been largely unchanged for decades, relying on broad-acting immunosuppressants, in recent years multiple therapies employing systemic administration of antibodies targeting various aspects of the complex immune activation of atopic dermatitis (such as antibodies targeting the IL-4 (e.g., dupilumab), IL-12/23p40 (e.g., ustekinumab), IL-13 (e.g., tralokinumab, lebrikizumab), IL-17 (e.g., secukinumab), and IL-31 (e.g., nemolizumab) pathways) have been explored in the clinical setting. In fact, a systemic therapy (bi-weekly subcutaneous injections) using the monoclonal antibody dupilumab (anti-IL-4Ra) was recently approved by the FDA for the treatment of atopic dermatitis. However, systemic administration of therapeutic antibodies targeting immune pathways have been shown to repress the immune system, exposing the patient to significant risk of infections and other complications.

The topical application of vitamin D3, or its synthetic analogs, induces AD-like inflammation in mouse skin. The skin inflammation model induced by the vitamin D analog calcipotriol (MC903) has recently gained increased attention, as topical application of MC903 induces high levels of TSLP and the infiltration of group 2 (IL-5+ and IL-13+) innate lymphoid cells to the skin, thereby resembling some immune perturbations observed in skin lesions of humans with AD. Topical MC903 administration has also been shown to induce increased IL-4 signaling in treated animals (Martel et al., Yale J Biol Med (2017)).

The goals of the in vivo experiments described here were: (1) to establish a mouse MC903-induced model of atopic dermatitis and demonstrate the feasibility of expressing an HSV-encoded antibody when the virus was topically administered to AD-like skin in these animals; and (2) to assess whether a mouse surrogate single-chain antibody of dupilumab (anti-IL-4Ra) would provide relief to one or more AD symptoms in this animal model.

Materials and Methods

For nucleic acid analysis, skin biopsies were lysed in Buffer RLT (AllPrep DNA/RNA Mini Kit, Qiagen, cat. no. 80204) with DTT (G-Biosciences, cat. no. 3483-12-3) using sonication with a QSONICA sonicator (125 W, 20 Hz) at an amplitude of 25%. Following sonication, high speed centrifugation was used to pellet any insoluble material. DNA and RNA were isolated using an AllPrep DNA/RNA Mini kit (Qiagen) according to the manufacturer's protocol. For qPCR/qRT-PCR analysis, 50 ng of DNA or RNA were used per reaction in a total reaction volume of 25 μL. DNA quantification was determined by qPCR analysis using a Taqman® Fast Advanced Master Mix (Applied Biosystems); RNA quantification was determined by qRT-PCR analysis using Quantabio 1-Step RT-qPCR ToughMix. All samples were run in duplicate or triplicate.

For immunofluorescence, 5 μm sections were taken from OCT frozen tissue, mounted on slides, and air dried for up to 1 hour. The slides were then dipped in 100% methanol (MeOH) for 10 minutes at −20° C. and left to air dry. The methanol-fixed sections were rehydrated by washing 3 times in PBS (5 minutes each) at room temperature, followed by an incubation at room temperature in 3% H₂O₂ for 10 minutes, and three subsequent washes in PBS. The samples were then incubated with a blocking solution (Power Block) for 1 hour at room temperature in a humidified chamber. Excess blocking solution was removed, and the sections were stained with a drop of primary antibody (Ab) solution prepared in antibody diluent buffer (30-50 μL primary Ab solution/section). The sections were incubated with the primary antibody (mouse anti-human IgG antibody; Abcam, cat. no. ab200699) for 16 hours at 4° C. or 1 hour at room temperature, washed three times in TBST (TBS+0.025% Triton X-100) for 5 minutes at room temperature, and secondary Ab (Alexa Fluor® 488-conjugated goat anti-mouse antibody; ThermoFisher, cat. no. A-11029) was applied at a 1:200 dilution in antibody diluent buffer for 30 minutes at room temperature in a humidified chamber. Slides were once again washed three times with TB ST, and the stained sections were mounted with mounting media (ProLong™ Gold Antifade Mountant with DAPI, ThermoFisher, cat. no. P36931) and covered with a coverslip. The sections were imaged after dehydration (approximately 24 hours) using an ECHO Fluorescence Microscope.

For hematoxylin and eosin (H&E) staining, 5 μm sections were taken from cryopreserved tissue, mounted on slides, and air dried for up to 1 hour. The dried slides were rehydrated by soaking in double-distilled water for 2 minutes at room temperature. Sections were then incubated in Hematoxylin Gill 2× (VWR) for 2 minutes at room temperature, followed by being dipped 2 to 3 times in acid alcohol, dipped 3 to 4 times in Blue in Ammonia water, and incubated in eosin (Eosin Y Solution 1%, VWR) for 2 minutes. Samples were rinsed 3 to 4 times with tap water between each step. The stained and rinsed sections were gradually dehydrated with ethanol (EtOH) by first rinsing twice with 95% EtOH for 2 minutes each, then twice with 100% EtOH for 2 minutes each. Sections were then cleared through three rinses with Histo-Clear for 2 minutes each, mounted with mounting media (Permount™ Mounting Medium, @P15-100), and covered with a coverslip. The sections were imaged approximately 24 hours after dehydration using a brightfield microscope.

Results

### Establishment of an AD Animal Model

An in vivo study was initiated to establish that MC903 induced atopic dermatitis-like symptoms in treated animals. For this study, 14 C57BL/6J mice were used (two mice per group). MC903 was prepared in ethanol (EtOH) at a concentration of 100 μM. Mice were anaesthetized with isoflurane, their backs were shaved and treated with a chemical hair removal agent, and 25 μL of EtOH or MC903/EtOH was applied to the left and right ears (both sides) and to 4 marked dorsal sites (˜2 cm² each) on Day 1. The mice were then retreated with EtOH or MC903/EtOH at the same sites on Days 2, 3, 4, and 5. Next, select cohorts of animals received topical treatment with HSV-Ab1Fc1 (or vehicle control) formulated in a gel carrier to the left and right ears and 2 dorsal sites of MC903 treatment, and received an intradermal injection of HSV-Ab1Fc1 (or vehicle control) to the remaining 2 dorsal sites of MC903 treatment, on Day 5 or Day 7. These mice were then euthanized on Days 7 and 9, respectively. The HSV-AbFc1 vector (fully human anti-TNFα single-chain antibody) was used in this experiment as a proof-of-concept to assess antibody expression in AD-like lesions since this virus was appropriately purified and characterized at the time of study initiation. Table 6 provides a summary of the experimental design.

TABLE 6
summary of study design establishing MC903 model

| Grp | Sensitizing Agent (SA) | SA Site | Test Article (TA) | TA Site | Termination |
|---|---|---|---|---|---|
| 1 | EtOH | 2 ears, 4 dorsal | N/A | N/A | Day 7 |
| | (Days 1-5) | | N/A | N/A | |
| 2 | MC903/EtOH | 2 ears, 4 dorsal | N/A | N/A | Day 5 |
| | (Days 1-5) | | N/A | N/A | |
| 3 | MC903/EtOH | 2 ears, 4 dorsal | N/A | N/A | Day 7 |
| | (Days 1-5) | | N/A | N/A | |
| 4 | MC903/EtOH | 2 ears, 4 dorsal | Vehicle-topical | 2 ears, | Day 7 |
| | (Days 1-5) | | (Day 5) | 2 dorsal | |
| | | | Vehicle-intradermal | 2 dorsal | |
| | | | (Day 5) | | |
| 5 | MC903/EtOH | 2 ears, 4 dorsal | HSV-Ab1Fc1-topical | 2 ears, | Day 7 |
| | (Days 1-5) | | (Day 5) | 2 dorsal | |
| | | | HSV-Ab1Fc1-intradermal | 2 dorsal | |
| | | | (Day 5) | | |
| 6 | MC903/EtOH | 2 ears, 4 dorsal | Vehicle-topical | 2 ears, | Day 9 |
| | (Days 1-5) | | (Day 7) | 2 dorsal | |
| | | | Vehicle-intradermal | 2 dorsal | |
| | | | (Day 7) | | |
| 7 | MC903/EtOH | 2 ears, 4 dorsal | HSV-Ab1Fc1-topical | 2 ears, | Day 9 |
| | (Days 1-5) | | (Day 7) | 2 dorsal | |
| | | | HSV-Ab1Fc1-intradermal | 2 dorsal | |
| | | | (Day 7) | | |

At the indicated termination day, animals were euthanized, and the dorsal treatment sites were harvested using an 8 mm punch biopsy, while the ears were removed in their entirety. One half of each biopsy/ear was quick-frozen in liquid nitrogen for nucleic acid analysis, while the other half was processed for immunofluorescence analysis (as described above).

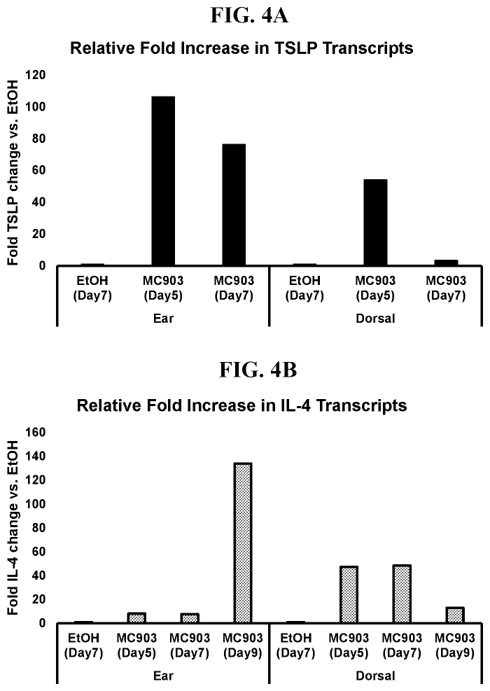

To confirm the appearance of atopic dermatitis-like lesions in mouse skin after topical MC903 treatment (Days 1-5), qRT-PCR analysis was conducted on harvested tissues to quantify changes in expression of certain markers of atopic dermatitis which had been previously shown by others to be upregulated in MC903-exposed skin. Specifically, the average fold change in GAPDH-corrected mouse TSLP or IL-4 transcripts in MC903-treated ear or dorsal tissues were calculated relative to the corresponding EtOH treated ear or dorsal tissues using the delta-delta Ct method. As observed previously by other groups, repeated topical MC903 exposure induced significant TSLP (FIG. 4A) and IL-4 (FIG. 4B) expression in treated mouse skin, confirming establishment of an AD-like mouse model. Interestingly, the kinetics of TSLP and IL-4 expression differed, with TSLP levels peaking on Day 5 in both ear and dorsal skin, while IL-4 expression had more delayed kinetics, showing the highest levels of transcript expression at Day 7 in dorsal skin and Day 9 in ear skin.

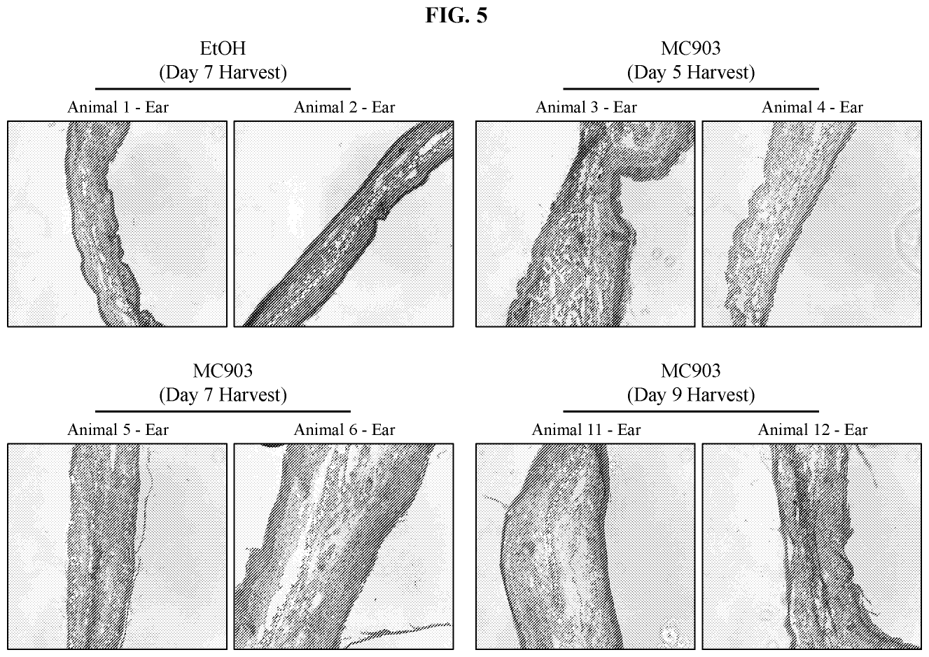

Paralleling the increased expression of TSLP and IL-4, a concomitant thickening of the ear skin was observed at the sites of MC903 treatment vs. ethanol control treatment (FIG. 5), further confirming that MC903 induced atopic dermatitis-like symptoms in this mouse model.

Next, the feasibility of expressing an HSV-encoded antibody after topical administration to an atopic dermatitis-like lesion was examined using an engineered virus (HSV-Ab1Fc1) encoding an exemplary, fully human single-chain antibody (Ab1Fc1). Ear and dorsal skin tissue sections were prepared for immunofluorescent analysis from animals exposed to MC903 on Days 1-5 and treated topically with HSV-Ab1Fc1 (compounded in a methylcellulose gel carrier) or vehicle control (compounded in a methylcellulose gel carrier) on Day 5 or Day 7. FIG. 6A shows representative immunofluorescence images of ear and dorsal skin harvested on Day 7 after topical treatment with HSV-Ab1Fc1 or vehicle control on Day 5. FIG. 6B shows representative immunofluorescence images of ear and dorsal skin harvested on Day 9 after topical treatment with HSV-Ab1Fc1 or vehicle control on Day 7. Robust human IgG protein expression was detected in both the ear and dorsal tissues infected with HSV-Ab1Fc1 but not vehicle control, suggesting that the virus was capable of delivering its encoded antibody cargo into atopic dermatitis-like lesions after topical exposure.

Taken together this data demonstrated the successful establishment of an AD-like phenotype upon MC903 treatment, and the feasibility of robust local delivery of a virally encoded antibody into an AD-like lesion after topical HSV administrations.

Assessment of Antibody Efficacy after Topical Herpes Virus Delivery

Due to the confirmed atopic dermatitis-like nature of the lesions induced by MC903 therapy (see e.g., FIG. 4), the efficacy of a topical HSV-encoded antibody to reduce one or more signs or symptoms of atopic dermatitis was next tested in this animal model. As noted above, dupilumab (an anti-human IL-4Ra antibody) is the only FDA-approved antibody for treating atopic dermatitis at present, which is administered systemically by bi-weekly subcutaneous injections. Here, a recombinant herpes virus (HSV-Ab66Fc1) was engineered to express a dupilumab surrogate antibody that targeted mouse IL-4Ra (Ab66Fc1, a mouse anti-mouse IL-4Ra single-chain antibody), and was tested for its ability to reduce MC903-mediated ear thickening. In addition, use of an antibody targeting the IL-4 pathway was chosen, in part, due to the observation that MC903 induced significant IL-4 signaling in C57BL/6J mice (FIG. 4B).

For this study, 8 C57BL/6J mice were used (two mice per group). MC903 was prepared in ethanol (EtOH) at a concentration of 100 μM. The backs of the mice were shaved with electric clippers and treated with a chemical hair removal compound on Day −2. On Day 1, mice were anaesthetized with isoflurane and 25 μL of EtOH or MC903/EtOH was applied to the left and right ears (both sides) and to 4 marked dorsal sites (˜2 cm² each). The mice were retreated with EtOH or MC903/EtOH at the same sites on Days 2, 3, 4, and 5 of the study. Next, select cohorts of animals received a total of five topical treatments with HSV-Ab66Fc1 or vehicle control formulated in a gel carrier to the left and right ears and 2 dorsal sites of EtOH or MC903/EtOH treatment, and received five total intradermal injections of HSV-Ab66Fc1 or vehicle control to the remaining 2 dorsal sites of EtOH or EtOH/MC903 treatment, per the study schedule. Table 6 provides a summary of the experimental design.

TABLE 6
summary of study design assessing HSV-encoded antibody efficacy

| | Sensitizing | Sensitization | | Test Article Treatment | | Termination |
|---|---|---|---|---|---|---|
| Grp | Agent | (Day) | Test Article | (Day) | #of sites | (Day) |
| 1 | EtOH | 1, 2, 3, 4, 5 | Vehicle | ears | 2 | 10 |
| | | | | 1, 3, 5, 7, 9 | (TOP) | |
| | | | | backs | 4 | |
| | | | | 1, 3, 5, 7, 9 | (2 TOP, 2 ID) | |
| 2 | MC903/EtOH | 1, 2, 3, 4, 5 | Vehicle | ears | 2 | 10 |
| | | | | 1, 3, 5, 7, 9 | (TOP) | |
| | | | | backs | 4 | |
| | | | | 1, 3, 5, 7, 9 | (2 TOP, 2 ID) | |
| 3 | EtOH | 1, 2, 3, 4, 5 | Ab66 | ears | 2 | 10 |
| | | | | 1, 3, 5, 7, 9 | (TOP) | |
| | | | | backs | 4 | |
| | | | | 1, 3, 5, 7, 9 | (2 TOP, 2 ID) | |
| 4 | MC903/EtOH | 1, 2, 3, 4, 5 | Ab66 | ears | 2 | 10 |
| | | | | 1, 3, 5, 7, 9 | (TOP) | |
| | | | | backs | 4 | |
| | | | | 1, 3, 5, 7, 9 | (2 TOP, 2 ID) | |

TOP = topical treatment;
ID = intradermal injection

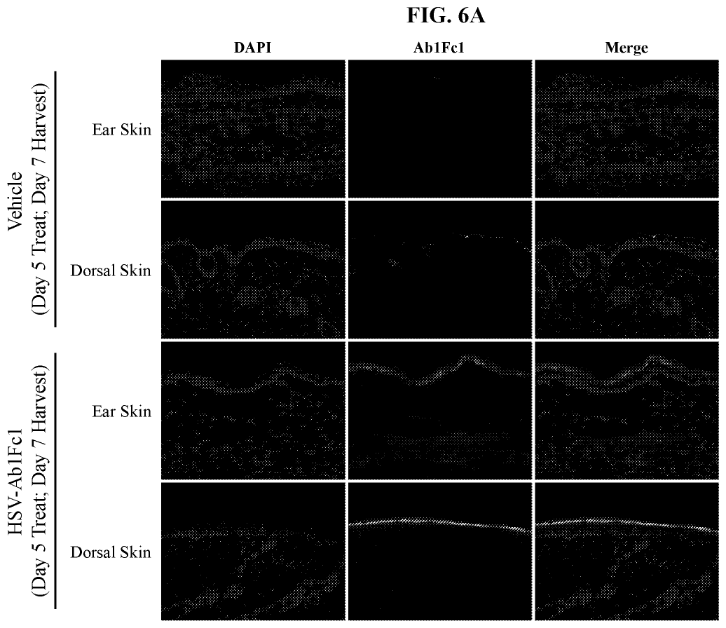

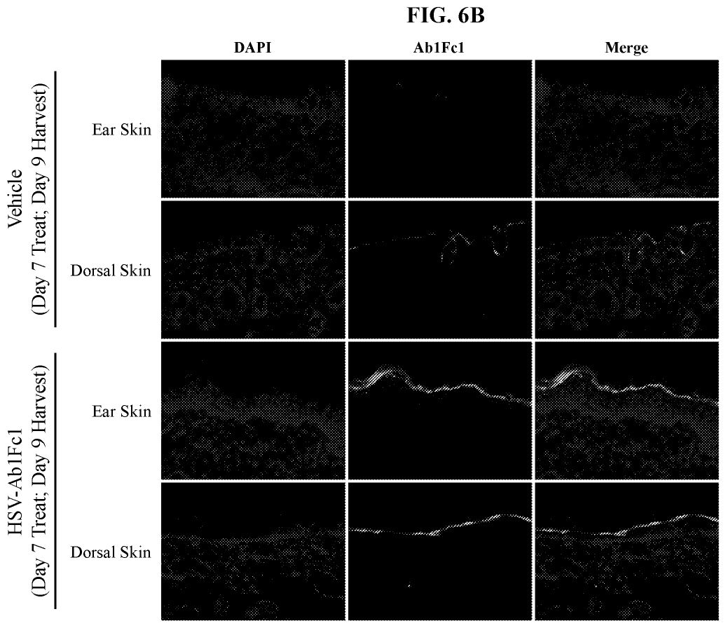

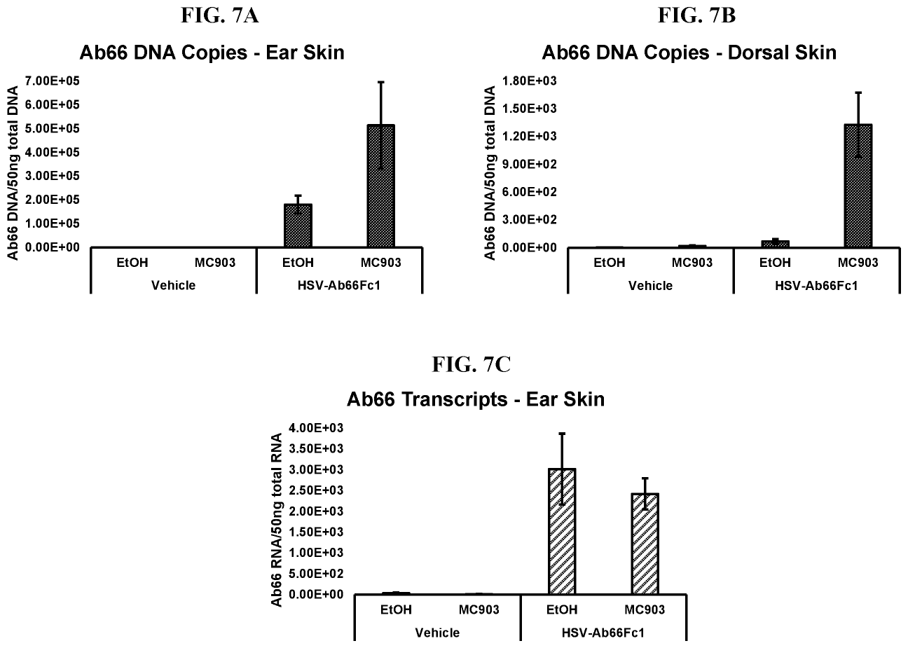

On Day 10, 8 mm punch biopsies were harvested from dorsal skin, while the ears were removed in their entirety, and the tissues were processed for nucleic acid analysis. qPCR data from the tissue harvests indicated that the engineered HSV genomes encoding the Ab66Fc1 transgene efficiently transduced MC903-treated ear skin (FIG. 7A), and, to a lesser extent, the MC903-treated dorsal skin (FIG. 7B) of immunocompetent animals Significant viral transduction was also observed in ethanol-treated ear skin, likely due to the thinness and fragility of this tissue type. Not only did the viral genomes efficiently transduce the ear skin, but the mouse anti-IL-4Ra single chain antibody was robustly expressed after infection, as assessed by qRT-PCR analysis (FIG. 7C) Minimal Ab66Fc1 transcripts were detected in the MC903-treated dorsal skin, paralleling the significantly lower transduction efficiency observed in this tissue, potentially due to the lesser MC903-induced skin compromise in this tissue type. No Ab66Fc1 DNA or RNA was detected in the vehicle control-treated tissues, indicating specificity of the assay for the single-chain antibody. Unfortunately, due to the single-chain antibody comprising a mouse IgG Fc, specific immunofluorescent detection of expressed protein was not possible in these samples due to endogenous mouse IgG.

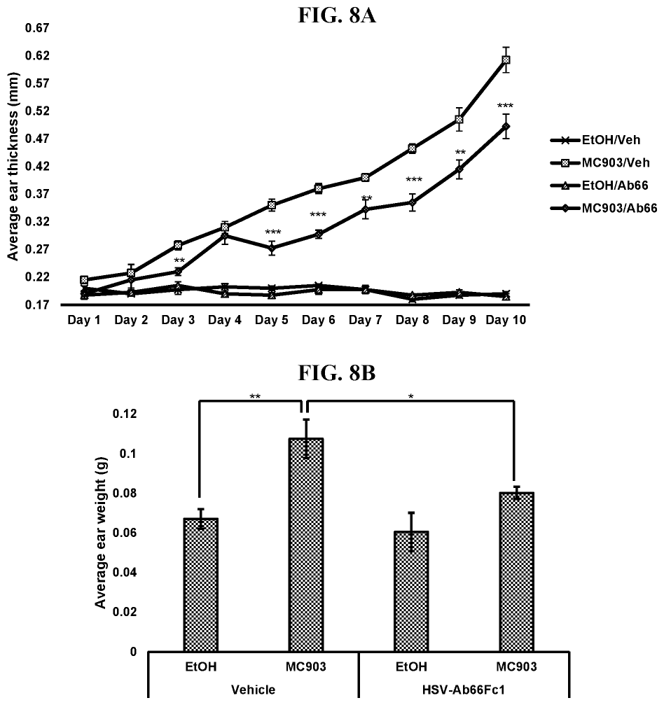

To assess efficacy of the topical therapy, ear thickness was measured for both ears on each mouse daily from Days 1-10 using a digital caliper (FIG. 8A). As expected, MC903 exposure induced significant ear thickening in treated mice, as compared to ethanol exposure. The average ear thickness at Day 10 for ethanol/vehicle treated animals was 0.19 mm, while the average thickness at this same time point for MC903/vehicle treated animals was 0.6125 mm. However, a significant reduction in ear thickening (>22% thinning) was observed by Day 5 in the MC903/Ab66Fc1 treated ears as compared to the MC903/vehicle treated ears, which carried through to the end of the experiment. The antibody caused no appreciable change in ear thickness of ethanol exposed ears (compare EtOH/veh to EtOH/Ab66 in FIG. 8A), confirming specificity of the antibody for reducing this atopic dermatitis-like phenotype.

Interestingly, the improvement in MC903-induced ear thickening at Day 5 mediated by topical treatment with HSV-Ab66Fc1 was comparable to the improvement observed at Day 5 in a parallel MC903 mouse model after administration of an anti-IL17c antibody (a pathway targeted by AD antibodies currently in human clinical trials (e.g., secukinumab)) published previously (see FIG. 5 of WO2017060289). However, while the positive results on ear thickness observed in the present study and those described in WO2017060289 were similar, the differences in the therapeutic approaches were stark. The study described in WO2017060289 required systemic administration of the therapeutic antibody via high dose intraperitoneal injection, and included an antibody pre-treatment step where the first dose of antibody was administered three days before MC903 treatment was initiated. The present example used local (as opposed to systemic) administration of the drug product, and did not employ any antibody pre-treatment (the first HSV-Ab66Fc1 application occurred after the first MC903 application).

At Day 10, the harvested ears from each mouse were weighed prior to any tissue manipulation. Paralleling the results from the ear thickness measures, MC903 treatment significantly increased average ear weight compared to ethanol control, which was partially rescued by HSV-Ab66Fc1 therapy (FIG. 8B). At the Day 10 harvest, it was also noted that the MC903/vehicle treated ears felt much stiffer/less malleable than the MC903/Ab66 treated ears, providing a qualitative measure of treatment efficacy.

Taken together, this data indicated that engineered recombinant herpes viruses could successfully express their encoded cargo in atopic dermatitis-like lesions after topical treatment, and further, expression of an antagonist antibody targeting a pathway known to play a role in human AD disease provided quantitative and qualitative improvements in the disease phenotype. Without wishing to be bound by theory, it is believed that localized administration of a herpes viral vector encoding an antibody provides a novel and unique method of delivering an effective immunotherapy (e. g., to treat inflammatory diseases/conditions like atopic dermatitis) while limiting systemic exposure to the antibody.

What is claimed is:

1. A recombinant herpes virus genome comprising one or more polynucleotides encoding an antibody.

2. The recombinant herpes virus genome of claim 1, wherein the antibody is an antibody fragment.

3. The recombinant herpes virus genome of claim 2, wherein the antibody fragment is a Fab, Fab′ Fab′-SH, F(ab′)2, Fv, scFv, or scFv-Fc fragment.

4. The recombinant herpes virus genome of claim 1, wherein the antibody is a single-domain antibody.

5. The recombinant herpes virus genome of claim 1, wherein the antibody is a full-length antibody.

6. The recombinant herpes virus genome of any one of claims 1-5, wherein the antibody is a murine antibody, a chimeric antibody, a humanized antibody, a human antibody, a monoclonal antibody, or a multispecific antibody.

7. The recombinant herpes virus genome of any one of claims 1-6, wherein the antibody is an IgA, IgD, IgE, IgG, or IgM antibody.

8. The recombinant herpes virus genome of any one of claims 1-7, wherein the antibody is an IgG antibody.

9. The recombinant herpes virus genome of claim 8, wherein the IgG antibody is an IgG1, IgG2, IgG3, or IgG4 antibody.

10. The recombinant herpes virus genome of claim 8 or claim 9, wherein the IgG antibody is an IgG1 antibody.

11. The recombinant herpes virus genome of claim 8 or claim 9, wherein the IgG antibody is an IgG4 antibody.

12. The recombinant herpes virus genome of any one of claims 1-11, wherein the antibody is an agonist antibody.

13. The recombinant herpes virus genome of any one of claims 1-11, wherein the antibody is an antagonist antibody.

14. The recombinant herpes virus genome of any one of claims 1-13, wherein the antibody comprises a heavy chain variable region comprising an HVR-H1, an HVR-H2, and an HVR-H3, wherein the HVR-H1 comprises a sequence selected from the group consisting of SEQ ID NOS: 1-59, the HVR-H2 comprises a sequence selected from the group consisting of SEQ ID NOS: 60-122, and/or the HVR-H3 comprises a sequence selected from the group consisting of SEQ ID NOS: 123-185.

15. The recombinant herpes virus genome of any one of claims 1-14, wherein the antibody comprises a light chain variable region comprising an HVR-L1, an HVR-L2, and an HVR-L3, wherein the HVR-L1 comprises a sequence selected from the group consisting of SEQ ID NOS: 186-242, the HVR-L2 comprises a sequence selected from the group consisting of SEQ ID NOS: 243-294, and/or the HVR-L3 comprises a sequence selected from the group consisting of SEQ ID NOS: 295-354.

16. The recombinant herpes virus genome of any one of claims 1-13, wherein the antibody comprises a heavy chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 355-419 or 614-865.

17. The recombinant herpes virus genome of any one of claims 1-13, wherein the antibody comprises a light chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 420-482 or 866-1116.

18. The recombinant herpes virus genome of any one of claims 1-13, wherein the antibody comprises: (a) a heavy chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 355-419 or 614-865; and (b) a light chain variable region comprising a sequence having at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 420-482 or 866-1116.

19. The recombinant herpes virus genome of any one of claims 1-13, wherein the antibody is selected from the group consisting of abagovomab, abciximab, abituzumab, abrezekimab, abrilumab, actoxumab, adalimumab, adecatumumab, aducanumab, afasevikumab, afelimomab, afutuzumab, alacizumab, alemtuzumab, alirocumab, altumomab, amatuximab, anatumomab, andecaliximab, anetumab, anifrolumab, anrukinzumab, apolizumab, aprutumab, arcitumomab, ascrinvacumab, aselizumab, atezolizumab, atinumab, atlizumab, atorolimumab, avelumab, azintuxizumab, bapineuzumab, basiliximab, bavituximab, bectumomab, begelomab, belantamab, belimumab, bemarituzumab, belimumab, bemaritzumab, benralizumab, berlimatoxumab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab, briakinumab, brodalumab, brolucizumab, brontictuzumab, burosumab, cabiralizumab, camidanlumab, camrelizumab, canakinumab, cantuzumab, caplacizumab, capromab, carlumab, carotuximab, catumaxomab, cedelizumab, cemiplimab, cergutuzumab, certolizumab, cetrelimab, cetuximab, cibisatamab, citatuzumab, cixutumumab, clazakizumab, clenoliximab, clivatuzumab, codrituzumab, cofetuzumab, coltuximab, conatumumab, concizumab, cosfroviximab, crenezumab, crizanlizumab, crotedumab, cusatuzumab, dacetuzumab, daclizumab, dalotuzumab, dapirolizumab, daratumumab, dectrekumab, demcizumab, denintuzumab, denosumab, depatuxizumab, derlotuximab, detumomab, dezamizumab, dinutuximab, diridavumab, domagrozumab, dorlimomab, drozitumab, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab, enavatuzumab, enfortumab, enlimomab, enoblituzumab, enokizumab, enoticumab, ensituximab, epitumomab, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, felvizumab fezakinumab, fibatuzumab, ficlatuzumab, figitumumab, firivumab, flanvotumab, fletikumab, flotetuzumab, fontolizumab, foralumab, foravirumab, fremanezumab, fresolimumab, frunevetmab, fulranumab, futuximab, galcanezumab, galiximab, gancotamab, ganitumab, gantenerumab, gatipotuzumab, gavilimomab, gedivumab, gemtuzumab, gevokizumab, gilvetmab, gimsilumab, girentuximab, glembatumumab, golimumab, gomiliximab, gosuranemab, guselkumab, ianalumab, ibalizumab, ibritumomab, icrucumab, idarucizumab, ifabotuzumab, igovomab, iladatuzumab, imalumab, imaprelimab, imciromab, imgatuzumab, inclacumab, indatuximab, indusatumab, inebilizumab, inflectra, infliximab, intetumumab, inolimomab, inotuzumab, ipilimumab, iratumumab, isatuximab, iscalimab, istiratumab, itolizumab, ixekizumab, keliximab, labetuzumab, lacnotuzumab, ladiratuzumab, lampalizumab, lanadelumab, landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, loncastuximab, losatuxizumab, lilotomab, lintuzumab, lirilumab, lodelcizumab, lokivetmab, lorvotuzumab, lucatumumab, lulizumab, lumiliximab, lumretuzumab, lupartumab, lutikizumab, mapatumumab, margetuximab, marstacimab, maslimomab, mavrilimumab, matuzumab, mepolizumab, metelimumab, milatuzumab, minretumomab, mirikizumab, mirvetuximab, mitumomab, modotuximab, mogamulizumab, monalizumab, morolimumab, mosunetuzumab, motavizumab, moxetumomab, nacolomab, namilumab, naptumomab, naratuximab, narnatumab, natalizumab, navicixizumab, navivumab, naxitamab, nebacumab, necitumumab, nemolizumab, nerelimomab, nesvacumab, netakimab, nimotuzumab, nirsevimab, nivolumab, nofetumomab, obiltoxaximab, obinutuzumab, ocaratuzumab, ocrelizumab, odulimomab, ofatumumab, olaratumab, oleclumab, olendalizumab, olokizumab, omalizumab, onartuzumab, ontuxizumab, onvatilimab, opicinumab, oportuzumab, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab, pintumomab, placulumab, plozalizumab, pogalizumab, polatuzumab, ponezumab, porgaviximab, prasinezumab, prezalizumab, priliximab, pritoxaximab, pritumumab, quilizumab, racotumomab, radretumab, rafivirumab, ralpancizumab, ramucirumab, ranevetmab, ranibizumab, raxibacumab, ravagalimab, ravulizumab, refanezumab, regavirumab, remtolumab, reslizumab, rilotumumab, rinucumab, risankizumab, rituximab, rivabazumab, robatumumab, roledumab, romilkimab, romosozumab, rontalizumab, rosmantuzumab, rovalpituzumab, rovelizumab, rozanolixizumab, ruplizumab, sacituzumab, samalizumab, samrotamab, sapelizumab, sarilumab, satralizumab, satumomab, secukinumab, selicrelumab, seribantumab, setoxaximab, setrusumab, sevirumab, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, sulesomab, suptavumab, sutimlimab, suvizumab, suvratoxumab, tabalumab, tacatuzumab, tadocizumab, talacotuzumab, talizumab, tamtuvetmab, tanezumab, taplitumomab, tarextumab, tavolimab, tefibazumab, telimomab, telisotuzumab, tenatumomab, teneliximab, teplizumab, tepoditamab, teprotumumab, tesidolumab, tetulomab, tezepelumab, tibulizumab, tildrakizumab, tigatuzumab, timigutuzumab, timolumab, tiragotumab, tislelizumab, tisotumab, tocilizumab, tomuzotuximab, toralizumab, tosatoxumab, tositumomab, tovetumab, tralokinumab, trastuzumab, tregalizumab, tremelimumab, trevogrumab, tucotuzumab, tuvirumab, ublituximab, ulocuplumab, urelumab, urtoxazumab, ustekinumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab, votumumab, vunakizumab, xentuzumab, zalutumumab, zanolimumab, zatuximab, zenocutuzumab, ziralimumab, zolbetuximab, and zolimomab.

20. The recombinant herpes virus genome of any one of claims 1-19, wherein the recombinant herpes virus genome is replication competent.

21. The recombinant herpes virus genome of any one of claims 1-19, wherein the recombinant herpes virus genome is replication defective.

22. The recombinant herpes virus genome of any one of claims 1-21, wherein the recombinant herpes virus genome is selected from the group consisting of a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any derivatives thereof.

23. The recombinant herpes virus genome of any one of claims 1-22, wherein the recombinant herpes virus genome is a recombinant herpes simplex virus genome.

24. The recombinant herpes virus genome of claim 23, wherein the recombinant herpes simplex virus genome is a recombinant type 1 herpes simplex virus (HSV-1) genome, a recombinant type 2 herpes simplex virus (HSV-2) genome, or any derivatives thereof.

25. The recombinant herpes virus genome of claim 23 or claim 24, wherein the recombinant herpes simplex virus genome is a recombinant type 1 herpes simplex virus (HSV-1) genome.

26. The recombinant herpes virus genome of any one of claims 22-25, wherein the recombinant herpes simplex virus genome comprises an inactivating mutation.

27. The recombinant herpes virus genome of claim 26, wherein the inactivating mutation is in a herpes simplex virus gene.

28. The recombinant herpes virus genome of claim 27, wherein the inactivating mutation is a deletion of the coding sequence of the herpes simplex virus gene.

29. The recombinant herpes virus genome of claim 27 or claim 28, wherein the herpes simplex virus gene is selected from the group consisting of Infected Cell Protein (ICP) 0, ICP4, ICP22, ICP27, ICP47, thymidine kinase (tk), Long Unique Region (UL) 41, and UL55.

30. The recombinant herpes virus genome of claim 29, wherein the recombinant herpes simplex virus genome comprises an inactivating mutation in one or both copies of the ICP4 gene.

31. The recombinant herpes virus genome of claim 29 or claim 30, wherein the recombinant herpes simplex virus genome comprises an inactivating mutation in the ICP22 gene.

32. The recombinant herpes virus genome of any one of claims 29-31, wherein the recombinant herpes simplex virus genome comprises an inactivating mutation in the UL41 gene.

33. The recombinant herpes virus genome of any one of claims 29-32, wherein the recombinant herpes simplex virus genome comprises an inactivating mutation in one or both copies of the ICP0 gene.

34. The recombinant herpes virus genome of any one of claims 29-33, wherein the recombinant herpes simplex virus genome comprises an inactivating mutation in the ICP27 gene.

35. The recombinant herpes virus genome of any one of claims 22-34, wherein the recombinant herpes simplex virus genome comprises the one or more polynucleotides encoding the antibody within one or both of the ICP4 viral gene loci.

36. The recombinant herpes virus genome of any one of claims 1-35, wherein the recombinant herpes virus genome has reduced cytotoxicity when introduced into a target cell, as compared to a corresponding wild-type herpes virus genome.

37. The recombinant herpes virus genome of claim 36, wherein the target cell is a human cell.

38. A herpes virus comprising the recombinant herpes virus genome of any one of claims 1-37.

39. The herpes virus of claim 38, wherein the herpes virus is replication competent.

40. The herpes virus of claim 38, wherein the herpes virus is replication defective.

41. The herpes virus of any one of claims 38-40, wherein the herpes virus has reduced cytotoxicity as compared to a corresponding wild-type herpes virus.

42. The herpes virus of any one of claims 38-41, wherein the herpes virus is selected from the group consisting of a herpes simplex virus, a varicella zoster virus, a human cytomegalovirus, a herpesvirus 6A, a herpesvirus 6B, a herpesvirus 7, and a Kaposi's sarcoma-associated herpesvirus.

43. The herpes virus of any one of claims 38-42, wherein the herpes virus is a herpes simplex virus.

44. The herpes virus of claim 43, wherein the herpes simplex virus is a type 1 herpes simplex virus (HSV-1), a type 2 herpes simplex virus (HSV-2), or any derivatives thereof.

45. The herpes virus of claim 43 or claim 44, wherein the herpes simplex virus is a type 1 herpes simplex virus (HSV-1).

46. A pharmaceutical composition comprising the recombinant herpes virus genome of any one of claims 1-37 or the herpes virus of any one of claims 38-45 and a pharmaceutically acceptable excipient.

47. The pharmaceutical composition of claim 46, wherein the pharmaceutical composition is suitable for topical, transdermal, subcutaneous, intradermal, transmucosal, oral, intranasal, intratracheal, sublingual, nasal, buccal, rectal, vaginal, intravenous, intraarterial, intramuscular, intracardiac, intraosseous, intraperitoneal, intraorbital, intravitreal, subconjunctival, suprachoroidal, subretinal, intraarticular, peri-articular, local, epicutaneous, and/or inhaled administration.

48. The pharmaceutical composition of claim 46 or claim 47, wherein the pharmaceutical composition is suitable for topical administration.

49. The herpes virus of any one of claims 38-45 or the pharmaceutical composition of any one of claims 46-48 for use as a medicament.

50. The herpes virus of any one of claims 38-45 or the pharmaceutical composition of any one of claims 46-48 for use in a therapy.

51. Use of the herpes virus of any one of claims 38-45 or the pharmaceutical composition of any one of claims 46-48 in the manufacture of a medicament for treating a disease.

52. The use of claim 51, wherein the disease is selected from the group consisting of psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer, hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, an autoimmune disease, asthma, thyroid eye disease, an infectious disease, and a neurological disease.

53. A method of administering an antibody to a subject, the method comprising administering to the subject an effective amount of the herpes virus of any one of claims 38-45 or the pharmaceutical composition of any one of claims 46-48.

54. A method of providing prophylactic, palliative, and/or therapeutic relief of one or more signs or symptoms of a disease in a subject, the method comprising administering to the subject an effective amount of the herpes virus of any one of claims 38-45 or the pharmaceutical composition of any one of claims 46-48.

55. The method of claim 53 or claim 54, wherein the herpes virus or pharmaceutical composition is administered topically, transdermally, subcutaneously, intradermally, transmucosally, orally, intranasally, intratracheally, sublingually, nasally, buccally, rectally, vaginally, intravenously, intraarterially, intramuscularly, intracardially, intraosseously, intraperitoneally, intraorbitally, intravitreally, subconjunctivally, suprachoroidally, subretinally, intraarticularly, peri-articularly, locally, epicutaneously, or via inhalation.

56. The method of claim 54 or claim 55, wherein the disease is selected from the group consisting of psoriasis, atopic dermatitis, pyoderma gangrenosum, a blistering disease, pemphigus, pemphigus vulgaris, pemphigus foliaceus, an autoimmune bullous skin disorder, bullous pemphigoid, Behçet's disease, cancer, hidradenitis suppurativa, arthritis, rheumatoid arthritis, psoriatic arthritis, osteoarthritis, juvenile idiopathic arthritis, ankylosing spondylitis, axial spondylarthritis, reactive arthritis, enteropathic arthritis, asthma, an autoimmune disease, thyroid eye disease, an infectious disease, and a neurological disease.

57. A method of administering an antibody to the epidermis and/or dermis of a subject, the method comprising topically, transdermally, or intradermally administering to the subject an effective amount of the herpes virus of any one of claims 38-45 or the pharmaceutical composition of any one of claims 46-48.

58. The method of claim 57, wherein the skin of the subject is abraded prior to administration.

59. A method of administering an antibody to the mucosa of a subject, the method comprising topically, transmucosally, orally, sublingually, nasally, intranasally, via inhalation, or buccally administering to the subject an effective amount of the herpes virus of any one of claims 38-45 or the pharmaceutical composition of any one of claims 46-48.

60. A method of administering an antibody to the airway or lungs of a subject, the method comprising orally, sublingually, nasally, intranasally, intratracheally, via inhalation, or buccally administering to the subject an effective amount of the herpes virus of any one of claims 38-45 or the pharmaceutical composition of any one of claims 46-48.

61. A method of administering an antibody to one or more joints of a subject, the method comprising intraarticularly administering to the subject an effective amount of the herpes virus of any one of claims 38-45 or the pharmaceutical composition of any one of claims 46-48.

62. A method of administering an antibody to one or both eyes of a subject, the method comprising topically, intraorbitally, intravitreally, subconjunctivally, subretinally, or suprachoroidally administering to the subject an effective amount of the herpes virus of any one of claims 38-45 or the pharmaceutical composition of any one of claims 46-48.

63. The method of any one of claims 53-62, wherein the subject is a human.

64. The method of any one of claims 53-63, wherein the subject is not exposed to the antibody systemically.
